(19) United States
(12) Patent Application Publication
(10) Pub. No.: US 2011/0262921 A1
(43) Pub. Date: Oct. 27, 2011

(54) Test for the Detection of Bladder Cancer

(76) Inventors: Anita L. Sabichi, Houston, TX (US); John L. Clifford, Shreveport, LA (US); Randolph Stone, JR., Shreveport, LA (US)

(21) Appl. No.: 13/091,961

(22) Filed: Apr. 21, 2011

Related U.S. Application Data
(60) Provisional application No. 61/343,123, filed on Apr. 23, 2010.

(51) Int. Cl. C12Q 1/68; G01N 33/573; G01N 33/566

(52) U.S. Cl. 435/6.12; 435/7.92; 436/501; 435/7.4

(57) ABSTRACT

A diagnostic or prognostic method for detecting malignant and premalignant bladder cancer comprising the identification and quantification of an expression level of identified gene products in the body fluids of a patient and subsequently comparing the expression level of the patient to the expression level found in subjects that do not have bladder cancer.

### CROSS-REFERENCE TO RELATED APPLICATION

This application claims the benefit under 35 USC §119(e) to U.S. provisional application Ser. No. 61/343,123 filed Apr. 23, 2010.

### STATEMENT REGARDING FEDERALLY SPONSORED RESEARCH OR DEVELOPMENT

The invention described herein was made with support of the U.S. Government, and the U.S. Government may have certain rights in the invention as provided for by the terms of grant numbers R21 CA116324 and N01 CN 85186 awarded by the National Institutes of Health (NIH) and the National Cancer Institute (NCI).

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC

Not Applicable.

### BACKGROUND OF THE INVENTION

This invention provides biomarkers that can be used as diagnostics and/or prognostics for the detection of bladder cancer (also called urothelial cell carcinoma or UCC). More specifically, this invention describes gene products that have previously been shown to be detectable in urine or blood, which can be used in a simple diagnostic and/or prognostic test. These include gene products that are either up or down regulated and therefore would allow for the development of diagnostics that could control for protein concentration by including one or more from each class (up or down regulated) of gene product. Finally, gene products that are strongly upregulated are described that may represent markers for bladder premalignancy. Not only can noninvasive or minimally invasive diagnostic test be developed for assessing onset of bladder cancers, but such tests may also be used to determine the efficacy of a treatment. Furthermore, as the use of these tests in combination with various treatments occurs, a pattern of responsiveness of a treatment to specific gene product profiles may emerge allowing better choice of treatments. This invention can also be used to identify compounds that might be used to treat UCC by analyzing a compound's effect on gene expression patterns or by targeting a compound to a specific gene product to affect the change in said gene product associated with the disease. The compound can be a small molecule, an siRNA, a monoclonal antibody, a gene expression product or any other means of altering expression of the gene product. Finally, the biomarkers provided by the invention could be used as diagnostics and/or prognostics for screening at risk populations for UCC. At risk populations include current and former smokers and individuals exposed to occupational and environmental carcinogens.

Bladder cancer causes substantial morbidity and mortality and has the 4th highest incidence of all cancers in the developed world, with an estimated 70,530 new cases predicted to occur in the US in 2010 [Jemal A, Siegel R, Xu J, Ward E. Cancer Statistics, 2010. CA Cancer J Clin 2010; 60:277-300]. More than 90% of bladder tumors are urothelial cell carcinomas (UCCs). At the time of their diagnosis, approximately 75% are superficial tumors, 20% are invading muscular layers (infiltrating or invasive UCCs) and 5% are already metastatic. Of the superficial cases, approximately 20% are cured by means of a single surgical intervention, whereas between 50 and 70% recur one or more times after surgery, but never become infiltrating tumors. Between 10 and 30% of these superficial tumors become infiltrating tumors. These tumors are aggressive, poor-prognosis tumors with a mortality after 5 years of 50% and in the metastasized cases, the mortality after two years is 100% [Sanchez-Carbayo M, Socci N D, Charytonowicz E et al. Molecular profiling of bladder cancer using cDNA microarrays: defining histogenesis and biological phenotypes. Cancer Res 2002; 62:6973-80; Adshead J M, Kessling A M, Ogden C W. Genetic initiation, progression and prognostic markers in transitional cell carcinoma of the bladder: a summary of the structural and transcriptional changes, and the role of developmental genes. Br J Urol 1998; 82:503-12; Babaian R J, Johnson O F, Llamas L, Ayala A G. Metastases from transitional cell carcinoma of urinary bladder. Urology 1980; 16:142-44]. The majority of newly diagnosed cases (˜⅔) are confined to the urothelium (do not breach the lamina propria) and are hence “superficial” or superficially invasive [Prout G R, Jr. Bladder carcinoma and a TNM system of classification. J Urol 1977; 117:583-90].

Current diagnosis systems are based on a combination of urinary cytology (from squamous cells in urine) and of the direct observation of the bladder by means of cystoscopy. The latter is actually the main diagnostic and follow-up technique for tumors. It is performed by transurethral route, therefore it is an invasive and rather unpleasant technique for the patients. The sensitivity and specificity of this technique were believed to be quite high, although improvements in the actual technique (fluorescence cystoscopy) indicate that this is probably not so and that part of the recurrence observed in superficial tumors could be due to the lack of total resection in non-visible parts thereof [Jones J S. DNA-based molecular cytology for bladder cancer surveillance. Urology 2006; 67:35-45].

Urinary cytology is in turn a non-invasive diagnostic technique with a high sensitivity and specificity for high-grade tumors. However, this technique shows limitations for detecting low-grade tumors [Bastacky S, Ibrahim S, Wilczynski S P, Murphy W M. The accuracy of urinary cytology in daily practice. Cancer 1999; 87:118-28]. Cytologic abnormalities of the urothelium are associated with carcinoma in situ (CIS), and urine cytology is positive in 90% of cases because of cell shedding into the urine due to loss of cellular adhesiveness. Also, CIS is frequently associated with synchronous urothelial tumors of any stage. Strong indirect evidence indicates this lesion is a likely precursor of invasive carcinoma, but direct evidence in humans is lacking [Bostwick D G, Ramnani D, Cheng L. Diagnosis and grading of bladder cancer and associated lesions. Urol Clin North Am 1999; 26:493-507]. Urothelial CIS has a high likelihood (>80%) of progressing to invasive carcinoma if left untreated. Patients with UCC require frequent cystoscopic examination. If a tumor is found, treatment is transurethral resection (TUR) and intravesical treatment. Cystectomy is required for invasive UCC confined to the bladder [Sabichi A L, Lerner S P, Grossman H B, Lippman S M. Retinoids in the chemoprevention of bladder cancer. Curr Opin Oncol 1998; 10:479-84]. Traditional prognostic factors (tumor stage, and grade) do not sufficiently predict disease course or prognosis in the individual patient. Long-term study results clearly indicate that the ability to intervene at early stages and to monitor the success of treatment requires the definition of early markers for bladder cancer. Furthermore, the interpretation of the cytology is highly observer-dependent, therefore there may be inter-observer differences, especially in low-grade tumors.

All these limitations have led to the search for more reliable non-invasive bladder cancer markers. There are a few commercially available urine based tests for screening and surveillance for bladder cancer, but none of these can detect premalignancy [Budman L I, Kassouf W, Steinberg J R. Biomarkers for detection and surveillance of bladder cancer. Can Urol Assoc J 2008; 2:212-21; Konety B, Lotan Y. Urothelial bladder cancer: biomarkers for detection and screening. BJU Int 2008; 102:1234-41]. Finding a non-invasive marker with a high sensitivity and specificity for bladder UCC would be very helpful for clinical practice. In fact, several studies describe new tumor markers in urine, such as the test for the bladder tumor antigen NMP22 [Wiener H G, Mian C, Haitel A, Pycha A, Schatzl G, Marberger M. Can urine bound diagnostic tests replace cystoscopy in the management of bladder cancer? J Urol 1998; 159:1876-80; Soloway M S, Briggman V, Carpinito G A et al. Use of a new tumor marker, urinary NMP22, in the detection of occult or rapidly recurring transitional cell carcinoma of the urinary tract following surgical treatment. J Urol 1996; 156:363-67], fibrin degradation products [Schmetter B S, Habicht K K, Lamm D L et al. A multicenter trial evaluation of the fibrin/fibrinogen degradation products test for detection and monitoring of bladder cancer. J Urol 1997; 158:801-5.], telomerase [Takihana Y, Tsuchida T, Fukasawa M, Araki I, Tanabe N, Takeda M. Real-time quantitative analysis for human telomerase reverse transcriptase mRNA and human telomerase RNA component mRNA expressions as markers for clinicopathologic parameters in urinary bladder cancer. Int J Urol 2006; 13:401-8], tests based on fluorescent in situ hybridization [Hailing K C, King W, Sokolova I A et al. A comparison of BTA stat, hemoglobin dipstick, telomerase and Vysis UroVysion assays for the detection of urothelial carcinoma in urine. J Urol 2002; 167:2001-6] or flow cytometry [Takahashi C, Miyagawa I, Kumano S, Oshimura M. Detection of telomerase activity in prostate cancer by needle biopsy. Eur Urol 1997; 32:494-98; Trott P A, Edwards L. Comparison of bladder washings and urine cytology in the diagnosis of bladder cancer. J Urol 1973; 110:664-66], but although most of them have a higher sensitivity than urinary cytology, the latter is still the most specific [Bassi P, De M, V, De Lisa A et al. Non-invasive diagnostic tests for bladder cancer: a review of the literature. Urol Int 2005; 75:193-200].

There has also been extensive effort in recent years aimed at identifying new genetic markers, small biomolecules, and proteins, as biomarkers for bladder cancer diagnosis, prediction of recurrence, as well as for surrogate endpoints in chemoprevention trials [Budman LI, Kassouf W, Steinberg JR. Biomarkers for detection and surveillance of bladder cancer. Can Urol Assoc J 2008; 2:212-21; Lodde M, Fradet Y. The detection of genetic markers of bladder cancer in urine and serum. Curr Opin Urol 2008; 18:499-503]. Ideally such biomarkers should be detectable by non-invasive means, should be accurate, sensitive and provide a viable alternative to cystoscopy, which is invasive and can have a sensitivity as low as 70% [Lam T, Nabi G. Potential of urinary biomarkers in early bladder cancer diagnosis. Expert Rev Anticancer Ther 2007; 7:1105-15]. In spite of the identification of several promising markers by several groups, none have yet been able meet these criteria [Shariat S F, Karakiewicz P I, Ashfaq R, et al. Multiple biomarkers improve prediction of bladder cancer recurrence and mortality in patients undergoing cystectomy. Cancer 2008; 112:315-25; Harris L D, De La Cerda J, Tuziak T, et al. Analysis of the expression of biomarkers in urinary bladder cancer using a tissue microarray. Mol Carcinog 2008; 47:678-85; Alvarez A, Lokeshwar V B. Bladder cancer biomarkers: current developments and future implementation. Curr Opin Urol 2007; 17:341-6]. Large numbers of reports on expression results of all tumor types have started to appear in the literature, including bladder tumors [Sanchez-Carbayo M, Socci N D, Charytonowicz E et al. Molecular profiling of bladder cancer using cDNA microarrays: defining histogenesis and biological phenotypes. Cancer Res 2002; 62:6973-80; Ramaswamy S, Tamayo P, Rifkin R et al. Multiclass cancer diagnosis using tumor gene expression signatures. Proc Natl Acad Sci USA 2001. 98:15149-54; Sanchez-Carbayo M, Socci N D, Lozano J J et al. Gene discovery in bladder cancer progression using cDNA microarrays. Am J Pathol 2003; 163:505-16; Sanchez-Carbayo M, Capodieci P, Cordon-Cardo C. Tumor suppressor role of KiSS-1 in bladder cancer loss of KiSS-1 expression is associated with bladder cancer progression and clinical outcome. Am J Pathol 2003; 162:609-17; Dyrskjot L, Thykjaer T, Kruhoffer M et al. Identifying distinct classes of bladder carcinoma using microarrays. Nat Genet 2003; 33:90-96], although most of the results have not been made public in their entirety. However, up until now, the studies which have been conducted with specific bladder cancer markers have been focused on one or on very few genes [Olsburgh J, Hamden P, Weeks R et al. Uroplakin gene expression in normal human tissues and locally advanced bladder cancer. J Pathol 2003; 199:41-49; Fichera E, Liang S, Xu Z, Guo N, Mineo R, Fujita-Yamaguchi Y. A quantitative reverse transcription and polymerase chain reaction assay for human IGF-II allows direct comparison of IGF-II mRNA levels in cancerous breast, bladder, and prostate tissues. Growth Horm IGF Res 2000; 10:61-70; Simoneau M, Aboulkassim T O, LaRue H, Rousseau F, Fradet Y. Four tumor suppressor loci on chromosome 9q in bladder cancer: evidence for two novel candidate regions at 9q22.3 and 9831. Oncogene 1999; 18:157-63]. Recently, [Alcaraz Asensio A, Mengual Brichs L, Burset Albareda, M, Ribal Caparros M. J., Ars Criach E. Bladder Cancer Diagnosis and/or prognosis Method. U.S. patent application Ser. No. 12/532,139] 14 bladder tumor marker genes were identified and used to develop a bladder cancer diagnosis and prognosis method based on the detection and quantification of the gene expression of these genes by means of quantitative real-time PCR in RNA extracted from bladder fluids. However this method suffers the limitation of requiring sufficient quantities of intact RNA, which is often not possible for samples that have not been rapidly processed. Therefore the search for non-invasive biomarkers (ie. in urine) demands a more guided approach. To this end we have combined the UPII-SV40Tag mouse model for bladder cancer progression with Affymetrix microarray technology to determine the gene transcription profiles of urothelium from the UPII-SV40Tag mice and age matched non-transgenic littermates (WT), at different times during the course of tumor development.

In an attempt to generate a mouse model for bladder cancer progression, investigators in the laboratory of Xue-Ru Wu have engineered transgenic mice carrying a low copy number of the SV40 large T antigen (SV40Tag) oncogene, expressed under the control of the bladder urothelium specific murine uroplakin II promoter (UPII-SV40Tag mice) [Zhang Z T, Pak J, Shapiro E, Sun T T, Wu X R. Urothelium-specific expression of an oncogene in transgenic mice induced the formation of carcinoma in situ and invasive transitional cell carcinoma. Cancer Res 1999; 59:3512-7]. The SV40Tag oncogene can bind and inactivate the p53 and Rb tumor suppressor genes [Ahuja D, Saenz-Robles M T, Pipas J M. SV40 large T antigen targets multiple cellular pathways to elicit cellular transformation. Oncogene 2005; 24:7729-45], both of which are frequently mutated in human bladder UCC [Cordon-Cardo C, Zhang Z F, Dalbagni G, et al. Cooperative effects of p53 and pRB alterations in primary superficial bladder tumors. Cancer Res 1997; 57:1217-21]. In quiescent cells Rb is bound to E2F family transcription factors, suppressing their ability to activate transcription of genes required for DNA replication, nucleotide metabolism, DNA repair and cell cycle progression [Ahuja D, Saenz-Robles M T, Pipas J M. SV40 large T antigen targets multiple cellular pathways to elicit cellular transformation. Oncogene 2005; 24:7729-45; Dimova D K, Dyson N J. The E2F transcriptional network: old acquaintances with new faces. Oncogene 2005; 24:2810-26]. SV40T blocks the Rb-mediated repression of E2F proteins, thereby inducing expression of E2F-regulated genes such as cyclins E, A and D1, chk1, fen1, BRCA1 and many others. UPII-SV40Tag mice develop a condition closely resembling human CIS starting as early as 6 weeks of age. This condition eventually progresses to invasive UCC from 6 months of age onward. Histological examination of the bladder CIS lesions closely mimics the human histology [Zhang Z T, Pak J, Shapiro E, Sun T T, Wu X R. Urothelium-specific expression of an oncogene in transgenic mice induced the formation of carcinoma in situ and invasive transitional cell carcinoma. Cancer Res 1999; 59:3512-7].

Using this model, we have identified approximately 1,900 unique genes differentially expressed (≧3-fold difference at one or more time points) between WT and UPII-SV40Tag urothelium during the time course of tumor development. Among these, there were a high proportion of cell cycle regulatory genes and proliferation signaling genes that were more strongly expressed in the UPII-SV40Tag bladder urothelium.

### SUMMARY OF THE INVENTION

The invention features a diagnostic for detecting bladder cancer through noninvasive or minimally invasive techniques. More specifically, this invention provides a list of gene products that are increased or decreased in UCC and/or bladder premalignancy which can be used to develop diagnostics and/or prognostics for early detection of bladder cancer using urine or blood samples.

In one aspect the invention, ˜1,900 gene products that are either up or down regulated by at least three-fold in the mouse model are disclosed. Gene products that are upregulated during progression to malignancy include gene products encoding centromere proteins (Cenpa, Cenpf, Cenph; Aurora kinases A and B), cyclins (ccnb1, ccnb2, ccne2, ccna2 and ccnf); cell division cycle proteins (Cdc7, Cdc2a, Cdc20, Cdc6, Cdca3); kinesin-like family proteins, (kifc1, kif2c, kif11, kif20a, kif 22, kif23), multiple minichromosome maintenance deficient proteins (MCM2,4,5,6,7.8, and 10), and other proliferation related proteins such as E2f8, Spbc24, Top2a, Brca1, RacGAP1, RHAMM, and others. Gene products that are suppressed in mice with bladder carcinoma when compared to controls include extracellular matrix proteins (collagens Col1a1, Col1a2, Col6a2, Col3a1, laminin B1, and tenascin C), keratins (krt2-5, krt1-15 and other intermediate filament proteins) Dmn, Vim; as well as uroplakins upk1b and upk2, and other tight junction proteins (cldn8, ctnnb1, ctnnal1, ctnnd2, pcdhgc3, and cgnl1), superoxide dismutase 3 (SOD3), cyclin D2 (ccnd2), transthyretin (Ttr), bone morphogenetic protein 2 (BMP2), and matrix metalloproteinase 2 (Mmp2). One or more of these gene products can be used as a biomarker to detect changes indicative of premalignancy or malignancy. Table 1 provides a full list of such genes or gene products.

In yet another aspect of the foregoing invention, the genes or gene products can be used to define biological networks, which can be further used to develop diagnostics for bladder cancer progression. Thirty networks were identified that occurred at each of the time points assessed during tumor progression. Table 2 provides a list of all biological networks. Early events in cancer progression as observed in the top three biological networks centered on JUN, ERK, and P21. Said pathways that are identified early in the progression process (3 weeks) could be used to develop diagnostics for premalignancy. In addition, one or more of JUN, ERK and P21 genes or gene products can be used in diagnosis of premalignancy.

In the foregoing aspects of the invention, one or more of the gene products that are up regulated during premalignancy or malignancy can be detected with one or more of the gene products that are down regulated during premalignancy or malignancy. Detection of the gene products can be carried out using standard diagnostic methods such as q-rtPCR or ELISA. Other methods for detecting specific RNAs or proteins in blood or urine can also be used. By using a combination of gene products that are up regulated and down regulated during progression to cancer, better control for sample variability can be achieved. Fluids collected from a subject are used to detect and quantify the expression pattern of one or more gene products, then comparing the results obtained with normal reference values for said gene products in blood or urine.

In another aspect of this invention, the above described gene products (Table 1), biological networks (Table 2) and JUN, ERK, P21, can be used alone or in combinations to determine the efficacy of a treatment. A patient can be treated with, for example transurethral resection for superficial UCC followed by immunotherapy with Bacillus Calmette-Guérin (BCG), and a change in one or more gene products can be analyzed at various times following treatment to ensure that the desired efficacy is achieved.

In the foregoing aspect of the invention, said gene products can also be correlated with responsiveness to a given treatment such that this invention can be used to predict the best treatment for a patient. For example, analysis of treatment responsiveness may demonstrate that patients with changes in a subset of gene products at diagnosis respond better to treatment A, whereas patients with a different pattern of gene product changes respond better to treatment B.

Additionally, this invention can be used to find new molecules for treatment of bladder cancer. Screening can be carried out in cell lines or in animals that have altered expression of said gene products to identify compounds that can return expression levels back to normal or alter them in the direction of normal levels. Changes in key gene products associated with biological pathways can also be used for this purpose. Compounds can be small molecules, or to repress critical gene products in a pathway, siRNAs or monoclonal antibodies; or to enhance key gene products, a gene therapy expression system or a recombinant protein.

Finally, this invention can be used to provide diagnostics and/or prognostics for screening at risk populations for UCC, on a large scale.

By “biological pathway or pathway” is meant broadly as a group of functionally interrelated genes and gene products.

By “biomarker” is meant any RNA or protein that can be quantified from blood or urine fluids.

By “bladder cell carcinoma or bladder cancer” is meant any cancers that originate in the bladder.

By “blood samples” is meant any whole or part of material drawn from a patients vein, including whole blood, serum, or plasma.

By “compound” is meant any molecule, protein, antibody or nucleic acid that can be administered to a cell, animal or subject to assess changes in expression, tumorgenicity or phenotypic changes.

By “diagnostic test” is meant any kind of medical test performed to aid in the diagnosis or detection of disease.

By “down regulated” is meant a reduction in expression of a gene product in a bladder cancer subject by at least 3-fold from that found in non-bladder cancer patients.

By “gene products” is meant an mRNA or a protein encoded by a specific gene.

By “malignancy” is meant, in reference to a tumor that is cancerous, that can invade and destroy nearby tissue, and that may spread (metastasize) to other parts of the body.

By “premalignancy” is meant a disease, syndrome, or finding that, if left untreated, may lead to cancer.

By “up-regulated” is meant an increase in expression of a gene product in a bladder cancer subject by at least 3-fold from that found in non-bladder cancer patients.

By “urothelial cell carcinomas or UCCs” is meant cancers that originate in the cells that line the bladder called urothelial cells. Formerly called transitional cell carcinoma (TCC).

By “treatment or treating” is meant the medical management of a subject, e.g. an animal or human, with the intent that a prevention, cure, stabilization, or amelioration of the symptoms or condition will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder. “Treating” a condition with the compounds of the invention involves administering such a compound, alone or in combination and by any appropriate means, to an animal, cell, lysate or extract derived from a cell, or a molecule derived from a cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is H&E stain (panels a, d, and g) and MR images (b, c, e, f, h, and i) of the bladders of wild type littermates (WT, a-c) and transgenic mice at 6 weeks (d-f) and 38 weeks (gi) of age. Arrows indicate a thickened mucosa in early stage UCC (f) and invasion into surrounding tissue for advanced UCC (h, i). Panels a, d and g were photographed at 100× magnification.

FIG. 2 represents a preliminary hierarchical clustering of all four time points in the UPII-SV40Tag microarray analysis study: 3, 6, 20, and 30 weeks. The colors red, green, and black represent genes that are upregulated, downregulated, or no change, respectively, in the UPII-SV40Tag mice when compared to the WT littermates. The last cluster, cluster 8, has been enlarged and rescaled and represents genes that are highly upregulated at all four time points.

FIG. 3, panel A, selected genes that were expressed at higher levels in UPII-SV40Tag urothelium relative to WT littermates are shown. Black bars indicate raw expression values for UPII-SV40Tag mice (n=2). Open bars indicate expression values for WT mice.

FIG. 3, panel B are selected genes that were expressed at lower levels in UPII-SV40Tag urothelium relative to WT littermates.

FIG. 4 represents confirmation of microarray data for selected genes. RT-PCR was performed using the cDNA made from RNA extracted from the urothelium of 6 week old WT and 3 week old hemizygous mice (+/−) SV40Tag mice.

FIG. 5 are gene interaction networks #1-3. Gene expression profiles of 3 week old WT and UPII-SV40Tag bladder urothelial tissue (n=2 for each group) were compared using Ingenuity microarray analysis software. Genes overexpressed and underexpressed in UPII-SV40Tag bladders compared to WT mice, at the 3 week time point, are shown in red and green, respectively.

FIG. 6, panel A are differentially expressed genes in human bladder derived cells. Semiquantitative RT-PCR of 25, 30, and 35 cycles was performed on four human bladder cell lines using primers for four of the genes identified from the UPII-SV40Tag mouse microarray experiment. The relative expression was determined by a ratio of each sample to the most intense sample for that gene, which was assigned a value of one.

FIG. 6, panel B are immunohistochemical detection of differentially expressed genes in paraffin sections of superficial bladder UCC. Sections were probed with antibodies for the indicated proteins and detected colorimetrically using horseradish peroxidase-coupled secondary antibodies, except for the RacGAP1 20X and RHAMM 40X panels which were detected by fluorescently tagged secondary antibodies.

FIG. 7, The Kaplan-Meier estimates by RacGAP1/PCNA and RHAMM (also called Hmmr). Panel A are Kaplan-Meier estimates by level of combinations of RacGap and PCNA, where significant differences in recurrence-free survival can be found among the 4 subsets of patients (p-value=0.0071, log-rank test, 0=low 1=high). Panel B, shows the comparison of recurrence-free survival between RHAMM Low and High groups at visit 0 for male patients on treatment arm with tumor stage equal to T1 or TA and average tumor grade equal to 1, 2, or 3.

### DETAILED DESCRIPTION OF THE INVENTION

This invention describes a comprehensive gene expression profile of bladder premalignancy. This is part of our long term effort to identify biomarkers of the earliest stages bladder UCC, which could potentially predict occurrence and recurrence of bladder UCC. A more immediate aim of these studies is to identify potential biomarkers of early stage bladder UCC, which can be tested in patient urine, bladder wash and other tissue samples. We have identified approximately 1,900 genes that are differentially expressed (>3-fold higher or lower) between the bladder urothelium of UPII-SV40Tag mice and their age-matched WT littermates at ages that encompass early stage changes that precede the appearance of CIS (3 weeks), CIS (6 weeks), and early and later stage UCC (20 and 30 weeks, respectively).

A large proportion of the genes upregulated in the UPII-SV40Tag urothelium are cell cycle regulatory and proliferation signaling genes. Many of these genes are common with the SV40T/t-antigen cancer signature identified recently by the laboratory of J. E. Green and collaborators, [Deeb K K, Michalowska A M, Yoon C Y, et al. Identification of an integrated SV40 T/t-antigen cancer signature in aggressive human breast, prostate, and lung carcinomas with poor prognosis. Cancer Res 2007; 67:8065-80]. These investigators used DNA microarrays to compare three transgenic mouse models for breast, lung, and prostate cancer, all based on tissue specific expression of SV40Tag, and found a common set of differentially expressed genes that are involved in cell proliferation, DNA repair and apoptosis. Of the 119 genes that comprise this T/t-antigen signature, 73 are found in our list of ˜1,900 differentially expressed genes (61% identity), suggesting similarity between models. Most importantly, this same signature of genes was associated with the most aggressive tumor phenotype and poor prognosis in human breast, lung, and prostate cancer [Deeb K K, Michalowska A M, Yoon C Y, et al. Identification of an integrated SV40 T/t-antigen cancer signature in aggressive human breast, prostate, and lung carcinomas with poor prognosis. Cancer Res 2007; 67:8065-80]. Whether the same association exists with human bladder UCC remains to be determined. In addition to over-expressed genes, we have identified several genes that are suppressed in the UPII SV40Tag bladders relative to WT littermates. This includes structural and cell adhesion genes that appear to be related to the normal differentiated state of urothelium. These include genes encoding extracellular matrix proteins, intermediate filament proteins, as well as uroplakins and other tight junction proteins. Table 1 contains the full list of genes that were found to by differentially expressed (≧3-fold at one or more time points) between UPII-SV40Tag and WT littermate urothelium.

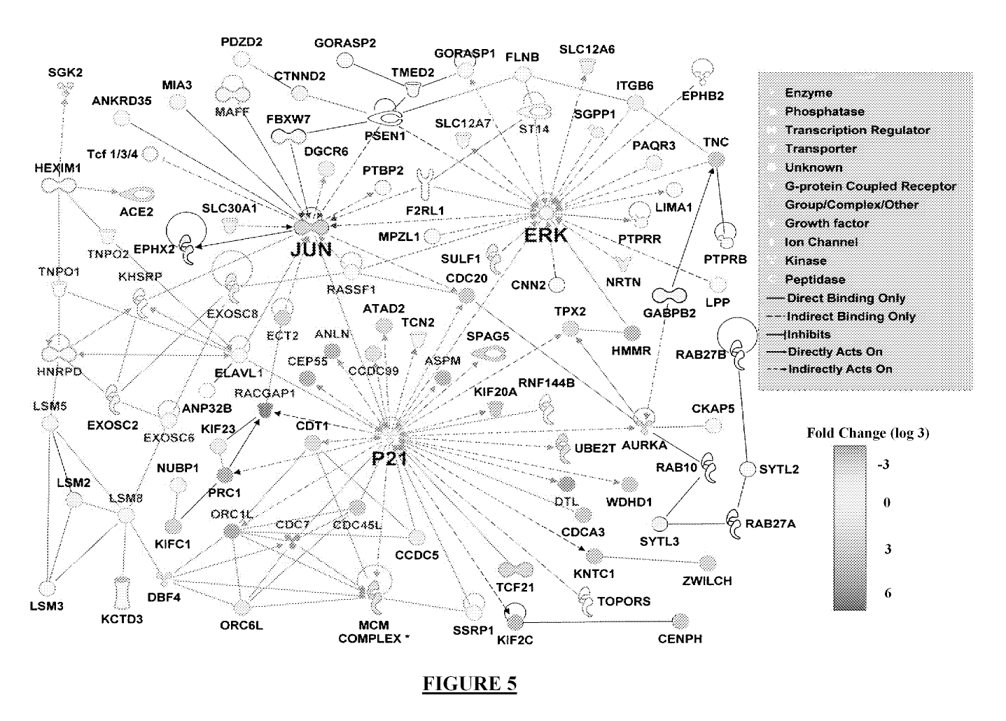

We used the Ingenuity Pathways Analysis software package to analyze the microarray data sets in order to identify the predominant cellular functions and signaling pathways that distinguish the earliest accessible stage of bladder UCC in the UPII-SV40T model. When we examined the biological networks that are derived from the 1,900 differentially expressed gene list, we noted that the top scoring networks contain genes involved in cell cycle, DNA replication, recombination, and repair, cancer, cellular movement, and cellular assembly and organization. The three highest scoring networks center on the activator protein 1(AP-1) transcription factor subunit, JUN; the MAP kinase extracellular signal-regulated kinase, ERK, and the cyclin dependent kinase inhibitor, P21 (FIG. 5). These are regulators of proliferative responses and are part of linked pathways all known to be affected by oncogenic mutations [Dhillon A S, Hagan S, Rath O, Kolch W. MAP kinase signalling pathways in cancer. Oncogene 2007; 26:3279-90]. AP-1 is a positive regulator of cell proliferation and transformation and its activity is stimulated in mouse skin tumorigenesis models by the tumor promoter TPA (12-O-tetradecanoylphorbol-13 acetate) [Angel P, Szabowski A, Schorpp-Kistner M. Function and regulation of AP-1 subunits in skin physiology and pathology. Oncogene 2001; 20:2413-23]. A direct link between between MAPK signaling and AP-1 activity has been established from studies in which kinase deficient forms of ERK could inhibit AP-1 activation by several stimuli [Frost J A, Geppert T D, Cobb M H, Feramisco J R. A requirement for extracellular signal-regulated kinase (ERK) function in the activation of AP-1 by Ha-Ras, phorbol 12- myristate 13-acetate, and serum. Proc Natl Acad Sci USA 1994; 91:3844-8; Watts R G, Huang C, Young M R, et al. Expression of dominant negative Erk2 inhibits AP-1 transactivation and neoplastic transformation. Oncogene 1998; 17:3493-8]. Those findings have led to several studies, including our own, aimed at understanding the mechanism of suppression of MAP kinase signaling and/or AP-1 activity by the vitamin A metabolite all-trans retinoic acid (ATRA), an efficient suppressor of tumor formation in several epithelial cancer models [Niles R M. Signaling pathways in retinoid chemoprevention and treatment of cancer. Mutat Res 2004; 555:81 96; Cheepala S B, Yin W, Syed Z, et al. Identification of the B-Raf/Mek/Erk MAP kinase pathway as a target for all-trans retinoic acid during skin cancer promotion. Mol Cancer 2009; 8:27; Cheepala S B, Syed Z, Trutschl M, Cvek U, Clifford J L. Retinoids and skin: Microarrays shed new light on chemopreventive action of all-trans retinoic acid. Mol Carcinog 2007; 46:634-9]. Our recent studies have shown that the B-Raf/Mek/Erk MAP kinase pathway is a target for the chemopreventive activity of ATRA in the mouse 2-stage skin carcinogenesis model. The present gene network analysis suggests that this same pathway could also be a target for prevention of bladder UCC, and will guide future experiments for testing potential chemopreventive and/or therapeutic drugs such as ATRA.

While any of the ˜1,900 gene products identified in this study (shown in Table 1) can be used alone or in combination with other gene products, including a combination that may include one or more gene products that have increased expression during bladder carcinoma with one or more gene products whose expression is decreased in bladder cancer patients, 962 were previously identified as bladder genes (Table 3), representing potential biomarkers for bladder carcinomas since their expression has already been linked to the bladder. To develop a non-invasive or minimally invasive diagnostic, gene products preferably would be found on the cell surface or would be secreted proteins. Of the identified gene products, 244 are known to be expressed on the cell surface and 119 are secreted. A total of 242 bladder-expressed gene products have previously been reported to be cell surface or secreted proteins (Table 3, gene products previously reported to be cell surface or secreted proteins denoted with a “*” symbol). These 242 gene products can be used alone or in combination with one or more of the other 242 gene products for the development of bladder carcinoma diagnostics and/or prognostics, preferably including one or more of the gene products whose expression is increased in bladder carcinoma together with one or more of the gene products whose expression is decreased in bladder carcinoma.

In addition to having cell surface expression or in being a secreted protein, a non-invasive or minimally invasive diagnostic should be readily assayed in bodily fluids, such as blood or urine. Of the ˜1,900 gene products identified, 229 have been previously detected in the urine and 552 in the blood. Of the 242 bladder cancer gene products known to be cell surface associated or secreted, 159 have previously been detected in the blood or serum (Table 4) and therefore could be used alone or in combination with one or more of the other 159 gene products for the development of bladder carcinoma diagnostics and/or prognostics, preferably including one or more of the gene products whose expression is increased in carcinoma together with one or more of the gene products whose expression is decreased in bladder carcinomas.

A similar analysis of gene products associated with bladder cancer was recently carried out [Alcaraz Asensio A, Mengual Brichs L, Burset Albareda, M, Ribal Caparros M. J., Ars Criach E. Bladder Cancer Diagnosis and/or prognosis Method. U.S. patent application Ser. No. 12/532,139]. In this study, 384 gene products were shown to be differentially expressed between tumor samples and control specimens. When these gene products were compared to the 159 bladder cancer gene products known to be cell surface associated or secreted and which have previously been detected in the blood or serum identified in this invention, only 17 were found to be in common. Thus, 142 new gene products (Table 5) have been identified that may be used alone or in combination with one or more of the other 142 novel gene products for the development of bladder carcinoma diagnostics and/or prognostics, preferably including one or more of the gene products whose expression is increased in carcinoma together with one or more of the gene products whose expression is decreased in bladder carcinomas. While the above represent the most current information on cell surface or secreted bladder cancer gene products found in the blood or urine, analysis of blood or urine samples can lead to identification of other gene products from the ˜1,900 identified in this study, which would also be useful in the practice of this invention. Similarly, additional bladder gene products from Table 1 may be identified as being expressed in the bladders from normal subjects or patients with bladder cancer, and would also be useful in the practice of this invention.

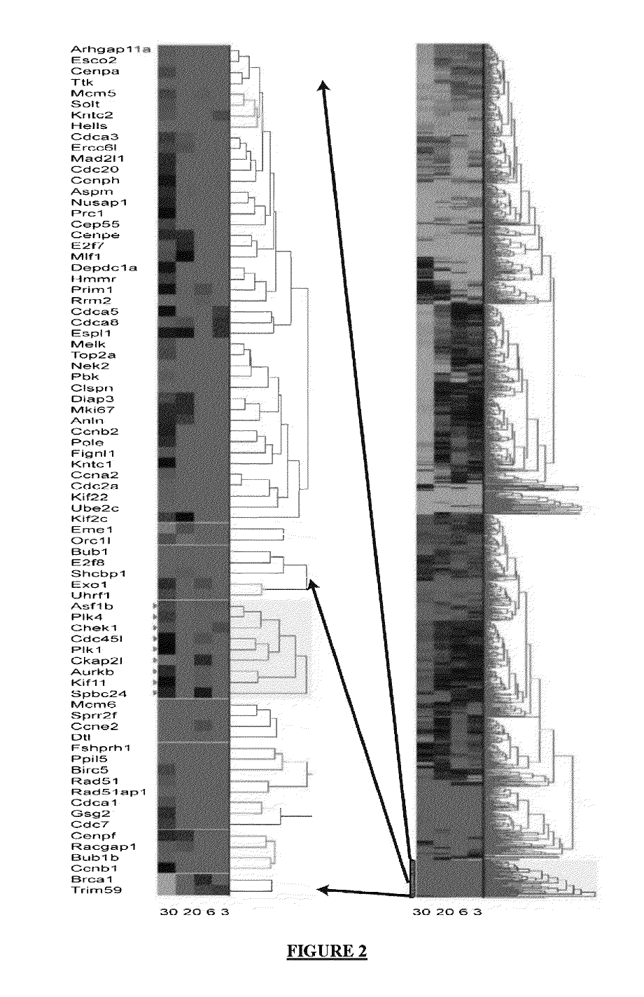

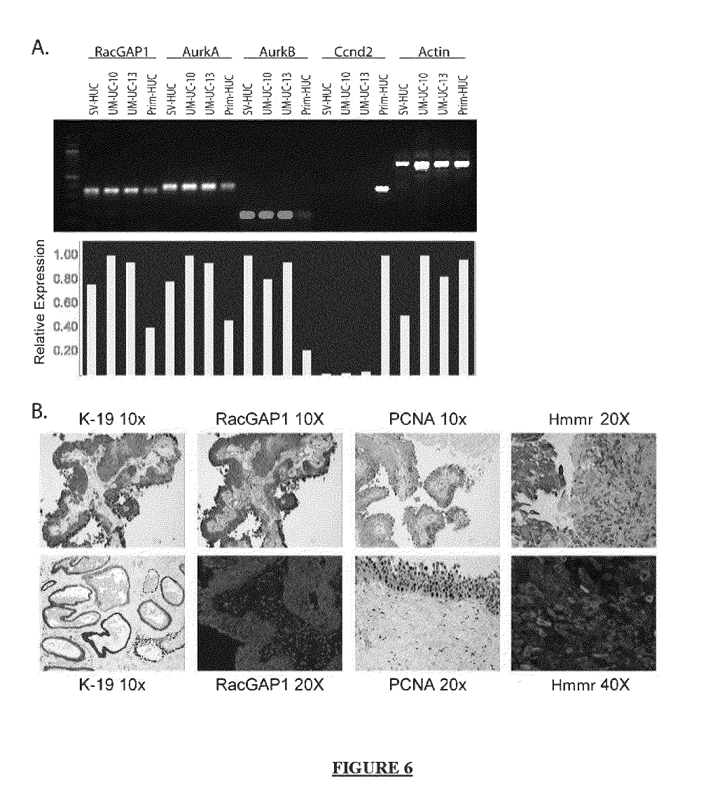

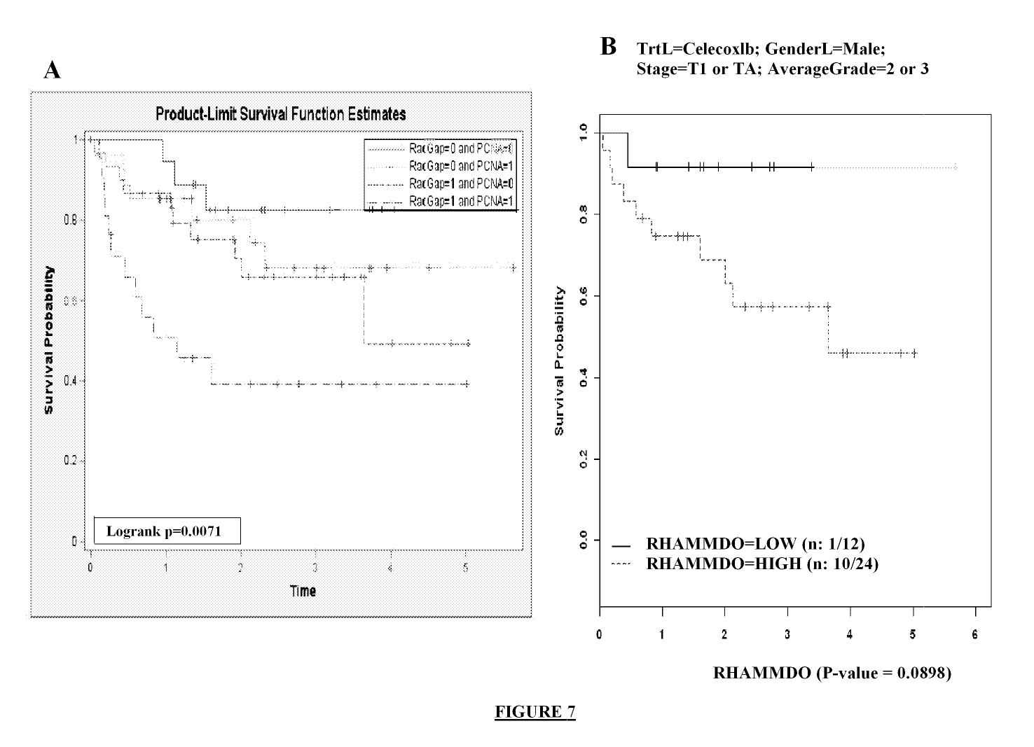

In an effort to narrow our candidate list of biomarkers to those most likely to be involved in the earliest stages of UCC, we have also focused on genes differentially expressed at the 3 week time point. Amongst these genes, we have further focused on those which also remain highly differentially expressed at later time points, with the expectation that such genes could also serve a markers for later stage UCC (FIG. 2, left panel). Also, in order to increase the likelihood of detection by antibodies in urine samples, we are paying closest attention to proteins that are secreted or are known to reside in the plasma membrane, and with some exceptions, those which have been previously detected in urine or bladder tissue by other investigators. These in include hyaluronan mediated motility receptor (Hmmr/RHAMM), which has been identified as highly expressed in early stage (Ta, T1), and later stage (T2-4) bladder UCC [Kong Q Y, Liu J, Chen X Y, Wang X W, Sun Y, Li H. Differential expression patterns of hyaluronan receptors CD44 and RHAMM in transitional cell carcinomas of urinary bladder. Oncol Rep 2003; 10:51-5]; proliferating cell nuclear antigen (PCNA) [Inagaki T, Ebisuno S, Uekado Y, et al. PCNA and p53 in urinary bladder cancer: correlation with histological findings and prognosis. Int J Urol 1997; 4:172-7], autocrine motility factor receptor (AMFR) [Korman H J, Peabody J O, Cerny J C, Farah R N, Yao J, Raz A. Autocrine motility factor receptor as a possible urine marker for transitional cell carcinoma of the bladder. J Urol 1996; 155:347-9] and others. We have tested for expression of several candidate genes that are upregulated in UPII-SV40Tag mice at all 4 time points (RacGAP1, PCNA, Survivin, and RHAMM), are upregulated and secreted (IL18) and are upregulated and expressed at the cell surface (PON3), in paraffin sections of high and low grade superficial bladder UCC samples. We detect all of these proteins to varying degrees in the tumor samples, with the highest levels detectable for RacGAP1, PCNA and RHAMM (FIG. 6B). This data, while preliminary, provides strong support for further testing and validation of these genes as biomarkers for superficial (early stage) UCC. We have also begun testing for expression of these proteins in urine samples from a recently completed Phase II, randomized, placebo controlled chemoprevention trial (N01 CN85186, PI: A. Sabichi) that was designed to test whether celecoxib can prevent recurrence in patients successfully treated by TUR for non-muscle invasive bladder cancer. In this trial, urine samples were collected over the course of treatment every 3 months for 18 months after curative therapy (TUR plus BCG), or until the time of recurrence (˜30% of patients). Analysis of urine samples for 99 patients demonstrates a correlation between high protein levels of RacGAP1 (data not shown) and a combination of RacGAP1 and PCNA, with recurrence (FIG. 7A). If patients had high levels of both RacGAP1 and PCNA their recurrence-free survival probability was about 40% (highly significant: Log-rank p=0.0071). We also found a subgroup of patients, Celecoxib treated males with any grade stage T1 or Ta bladder cancer, in which high levels of RHAMM correlated with recurrence (p=0.0898) (FIG. 7B). The data for the complete analysis of AMFR, RacGAP1, RHAMM, and PCNA for all 99 patients at different time points during the chemoprevention trial is shown in Table 6.

It is anticipated that single markers or combinations of markers can be validated for prediction of recurrence, and possibly for prediction of response to therapy. In the future we will also focus attention on proteins predicted to be downregulated in premalignant urothelium, such as uroplakin II, collagen 1A2 (Col1a2), bone morphogenetic protein 2 (BMP2), and superoxide dismutase 3 (SOD3). These could serve as negative markers for recurrence.

We have identified genes that are differentially expressed in premalignant urothelia, in a mouse model for aggressive bladder UCC. This group of genes now serves as a promising pool of candidates for biomarkers for early stage UCC, as well as a source for gaining insight into the earliest events preceding early stage UCC and/or CIS. These gene products can also be used for targeting therapies to protect against bladder cancer or for the treatment of bladder cancer. For example, one or more gene products in Table 1 that are overexpressed in bladder carcinomas could be used to screen for small molecules that suppress its (their) expression or inhibit its (their) activity. Similarly, monoclonal antibodies, siRNAs or antisense molecules can be developed to inhibit the activity of these over expressed gene products Likewise, therapeutic strategies such as recombinant proteins or gene therapy can be employed to compensate for one or more gene products that are inhibited in bladder carcinoma.

### Examples

### The UPII-SV40Tag Model Recapitulates Invasive Bladder UCC

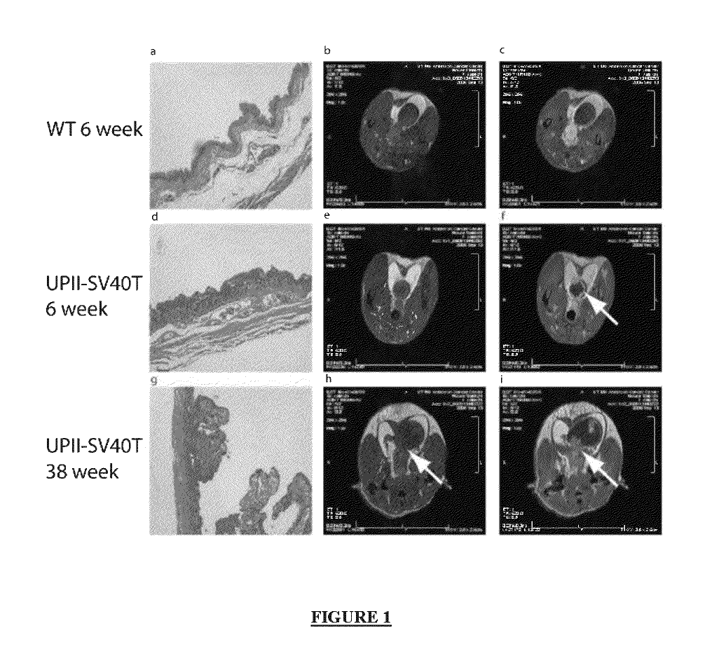

In this study we have used the UPII-SV40Tag mouse model of bladder cancer progression, in combination with comprehensive DNA microarray analyses, to explore early events in the development of bladder cancer, and to identify potential biomarkers for bladder premalignancy. An initial goal was to better characterize the earliest macroscopic changes in bladder tissue in live UPII-SV40Tag mice using small animal magnetic resonance (MR) imaging techniques developed at the MD Anderson Cancer Center's experimental animal imaging facility. We scanned and subsequently sacrificed mice at ages ranging from 6 weeks to 12 months of age, and compared MR image and histopathologic assessment of the genitourinary tract. An example of axial T1 postcontrast and T2 MR images of the urinary bladder of a 6 week old mouse that showed moderate irregular thickening of the urinary bladder mucosa which corresponded to histological findings of diffuse hyperplasia of the transitional epithelium is shown in FIG. 1 (FIG. 1d-f). At 38 weeks of age, MR imaging identified large, irregular contrast enhancing masses within the bladder lumen, some of which had invaded into the surrounding abdominal cavity (FIG. 1, h, i, arrows). These tumors were carcinoid in appearance and without the delicate papillae that characterize papillary tumors. We found a close correlation between MR image and histologic detection of intravesical abnormalities in the mice in all age groups (FIG. 1, compare a, d, and g to other panels).

### Gene Expression Profile of Bladder Cancer Progression

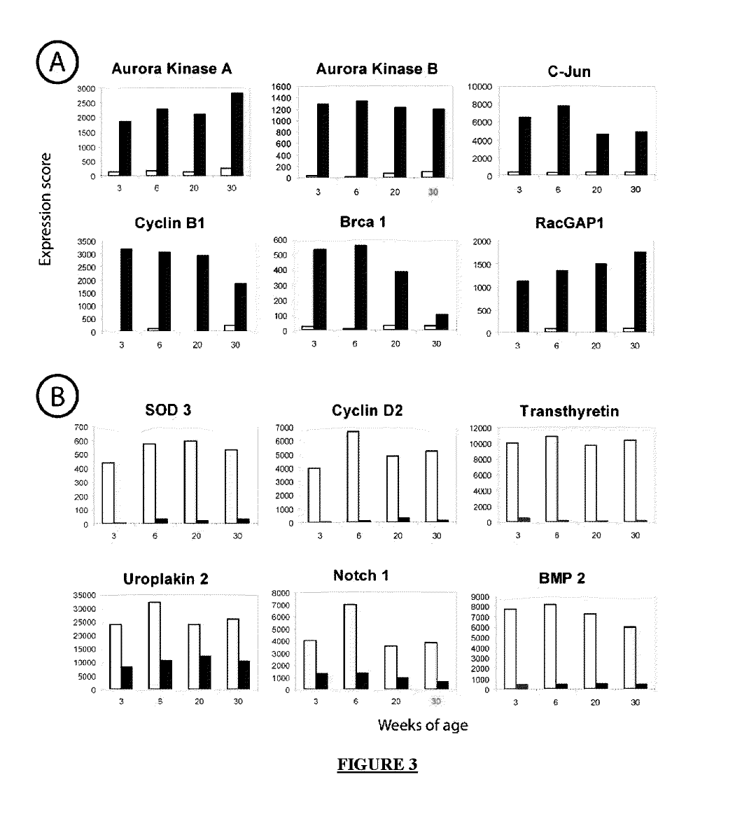

In parallel with the histologic and macroscopic characterization, we used Affymetrix DNA microarray technology to compare the gene transcription profiles of normal bladder urothelium (from non-transgenic littermates) with the urothelium of the UPII-SV40Tag mice, over time. We chose to examine mice at 3, 6, 20 and 30 weeks of age. These times for comparison encompass early stage changes that precede the appearance of CIS (3 weeks), CIS (6 weeks), and early and later stage UCC (20 and 30 weeks, respectively). Our determination of genes expressed in the urothelium at these time points revealed approximately 1,900 unique differentially expressed (≧3-fold difference) genes at one or more of the time points between the urothelium of UPII-SV40Tag mice and their age matched wild type (WT) littermates (see Table 1 for the full list). FIG. 2 illustrates the clustering of the differentially expressed genes according their expression patterns over the time course. Genes more highly expressed in the UPII-SV40Tag bladders are shown in red and genes more strongly expressed in WT bladders are shown in green. Black bars indicate genes that are expressed at similar levels in both mouse lines for that time point. We focused attention on a group of genes with a high fold increase in expression in UPII-SV40Tag mice for all 4 time points (FIG. 2, expanded left panel). We reason that this group of genes could contain candidate biomarkers for both premalignant and later stage UCC. The time course of expression of some of the most strongly upregulated and downregulated genes are shown graphically (FIG. 3A, higher expression in UPII-SV40Tag; FIG. 3B, lower expression in UPII-SV40Tag).

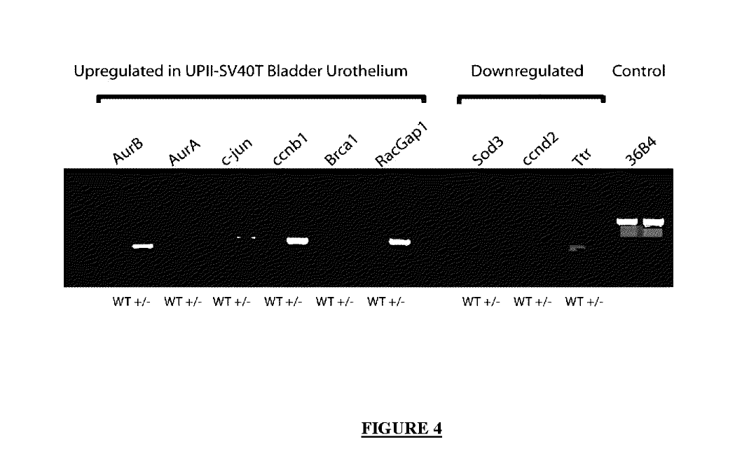

Interestingly some genes, like BRCA1, were strongly expressed in premalignant urothelium only at early stages of progression, with levels eventually dropping to near normal by later stages. We note a high proportion of genes involved in cell proliferation amongst the upregulated genes, and several structural and differentiation-related genes were among the downregulated genes. The microarray results were confirmed independently by RT-PCR for several of the genes (FIG. 4). For all genes tested to date, the relative direction of expression between WT and UPII-SV40Tag bladders was the same for both the RT-PCR and microarray results.

### Gene Network and Pathway Analysis

We next performed biometric analysis using the Path Explorer function in the Ingenuity Pathways Analysis software package (Ingenuity Systems Inc.) on the list of 1,900 differentially expressed genes (≧3-fold up or down at 1 or more time points), considering the expression differences between WT and UPII-SV40Tag mice separately for each time point. There was an average of 45 biological networks generated for the gene lists for each time point. Biological networks are defined as highly connected networks of up to 35 genes. A significance score based on a p-value calculation is assigned to each network and is displayed as a negative log of the p-value. The higher the score, the less likely it is that the set of genes from our list appearing in the network (focus genes) could be explained by random chance alone. See Table 2 for a full list of networks, their significance scores, number of focus molecules, relative direction of expression, and their top functions. The 30 top scoring gene networks were identical for all time points, although not all gene changes were the same in each network for each time point. This similarity between time points is expected since the same 1,900 gene list was used for each analysis, with only the fold difference between WT and UPIISV40Tag varying between sets. The similarity indicates that most of the major gene expression changes start to take place as early as 3 weeks. It should be noted that the 3 week time point precedes the appearance of invasive UCC by several weeks, such that gene expression differences at this time could be considered representative of a premalignant state. The top scoring networks contain genes involved in cell cycle, DNA replication, recombination, and repair, cancer, cellular movement, and cellular assembly and organization. The merged image of the top 3 networks for the 3 week time point indicates three nodes centered on JUN, ERK, and P21, all key regulators of proliferative responses (FIG. 5). Some of the other genes that are upregulated in UPII-SV40Tag mice include those encoding centromere proteins Cenpa, Cenpf, Cenph; Aurora kinases A and B; cyclins ccnb1, ccnb2, ccne2, ccna2 and ccnf; cell division cycle proteins Cdc7, Cdc2a, Cdc20, Cdc6, Cdca3; kinesin-like family proteins, kifc1, kif2c, kif11, kif20a, kif 22, kif23; multiple minichromosome maintenance deficient proteins MCM2,4,5,6,7.8, and 10; other proliferation related proteins such as E2f8, Spbc24, Top2a, Brca1, RacGAP1, RHAMM, and others (FIG. 2, FIG. 5, and Table 1). Many of these genes are common with the SV40T/t-antigen cancer signature identified recently by Deeb et al, for human breast, prostate, and lung carcinomas. In addition, we have identified several genes that are suppressed in the UPII-SV40Tag bladders relative to wild type littermates, which includes a large proportion of structural and cell adhesion genes that appear to be related to the normal differentiated state of urothelium. Examples are genes encoding extracellular matrix proteins such as collagens Col1a1, Col1a2, Col6a2, Col3a1, laminin B1, and tenascin C; keratins krt2-5, krt1-15 and other intermediate filament proteins Dmn, Vim; as well as uroplakins upk1b and upk2 itself, and other tight junction proteins cldn8, ctnnb1, ctnnal1, ctnnd2, pcdhgc3, and cgnl1. Other downregulated genes that are potentially involved in the development of UCC include superoxide dismutase 3 (SOD3), cyclin D2 (ccnd2), transthyretin (Ttr), bone morphogenetic protein 2 (BMP2), and matrix metalloproteinase 2 (Mmp2). As in FIG. 1, red and green indicates higher expression in the UPII-SV40Tag and WT bladders, respectively.

### Differentially Expressed Genes in Human Bladder-Derived Cell Lines and in Human Superficial Bladder Cancer

Finally, we have attempted to determine the relevance of several of the differentially expressed genes to human bladder cancer. We first compared mRNA expression levels for several genes in human normal urothelial cells (primary HUCs), ‘premalignant’ urothelial cells (SV-HUC) and advanced UCC cells (UM-UC-10, UM-UC-13). We have recently described the UM-UC cells in detail (21). The UM-UC-10 cells were derived from a bladder tumor, have mutant p53, undetectable levels of Rb, and are nontumorigenic in nude mice. The UM-UC13 cells were derived from a lymphatic metastasis, also have mutant p53 and undetectable RB, but are tumorigenic in nude mice. We observed that more than half of the genes tested by semi-quantitative RT-PCR were expressed as predicted in the human cell lines, such that genes overexpressed in the UPIISV40Tag mice were more strongly expressed in the premalignant and malignant cell lines (FIG. 6A and data not shown). Conversely, Ccnd2, which was downregulated in the UPII-SV40Tag mice, is expressed only in the primary HUCs.

Next we tested the expression of several differentially expressed genes in paraffin sections of high and low grade superficial bladder UCC samples that were excised by transurethral resection. We prioritized biomarkers for initial testing based on whether the genes were found to be highly expressed at all 4 time points (PCNA, Survivin, RHAMM, RacGAP1) as shown in FIG. 2, left panel; are cell surface proteins (PON3), or are secreted (IL18). We reason that such proteins would also have a higher likelihood of being detectable in urine. To date we have tested expression of 6 proteins in tumor samples from 12 patients (6 high grade and 6 low grade). All 6 of these proteins were detectable by immunohistochemical staining in the patient samples, with the strongest expression detected for RacGAP1, PCNA, and RHAMM (FIG. 6B, and data not shown). This expression also co-localized with expression of cytokeratin 19 (K19), a urothelial marker. RHAMM is expressed evenly throughout the cytoplasm, while PCNA is strongly expressed in the nuclei of hyperplastic urothelial cells, as previously described by others (FIG. 6B, lower row) [Kong Q Y, Liu J, Chen X Y, Wang X W, Sun Y, Li H. Differential expression patterns of hyaluronan receptors CD44 and RHAMM in transitional cell carcinomas of urinary bladder. Oncol Rep 2003; 10:51-5; Inagaki T, Ebisuno S, Uekado Y, et al. PCNA and p53 in urinary bladder cancer: correlation with histological findings and prognosis. Int J Urol 1997; 4:172-7]. We note that RacGAP1 is expressed in the cytoplasm, with prominent focal perinuclear staining, which is in agreement with our own immunocytochemical staining of bladder UCC cell lines (data not shown). It is not yet possible to determine whether there is a statistically significant difference in expression for any of the markers between high and low grade UCC due to the low sample size.

RHAMM, RacGAP1 and PCNA are potential urine biomarkers for premalignancy.

We have tested for expression of RacGAP1, RHAMM, and PCNA in urine samples from a recently completed Phase II, randomized, placebo controlled chemoprevention trial (N01 CN85186). That trial was designed to test whether Celecoxib, a selective COX-2 inhibitor, can prevent recurrence in patients successfully treated by TUR for non-muscle invasive bladder cancer. Urine samples were collected over the course of treatment every 3 months for 18 months after curative therapy (TUR and BCG), or until the time of recurrence (˜30% of patients). Analysis of urine samples for 99 patients demonstrates a correlation between high protein levels of RacGAP1 and PCNA at the baseline time point of the study (6 weeks after curative therapy), with recurrence (FIG. 7A). Over 60% of patients with high levels of both of these proteins had a recurrence of UCC within 18 months of curative therapy.

RHAMM expression at the baseline time point of the study also correlated with recurrence. However, this result was most striking in a subgroup of patients, the Celecoxib treated males with any grade stage T1 or Ta UCC (p=0.0898 comparing low to high RHAMM groups) (FIG. 7B). These results are promising and provide incentive for further testing of these biomarkers.

All publications, patents, and patent applications mentioned in this specification are herein incorporated by reference to the same extent as if each independent publication or patent application was specifically and individually indicated to be incorporated by reference. While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims. In short, it is the applicant's intention that the scope of the patent issuing herefrom will be limited only by the scope of the appended claims.

TABLE 1

| Gene Symbol | 3 wk | 1.5 mo | 5 mo | 7 mo |
|---|---|---|---|---|
| Aasdhppt | −0.5 | −0.3 | −0.7 | −1.16 |
| Aatf | −0.3 | 0 | −0.22 | −1.89 |
| Abca1 | −0.01 | 0.42 | −0.85 | −1.84 |
| Abca3 | −0.49 | −0.52 | −0.42 | −1.05 |
| Abcb8 | 0.33 | 0.7 | −0.04 | −1.32 |
| Abcc3 | −0.76 | −1.03 | −1.14 | −1.37 |
| Abhd10 | 0.67 | 1.32 | 0.6 | 0.7 |
| Abhd12 | −0.13 | 0.02 | −0.43 | −1.3 |
| Abhd14b | −1.3 | −0.88 | −0.66 | −0.74 |
| Abhd6 | 0.08 | −0.14 | −0.31 | −1.03 |
| Abhd8 | 0 | 0.37 | 0.95 | 1.06 |
| Abhd9 | 0 | 1.7 | 1.73 | 0.48 |
| Acaa1a /// Acaa1b | −0.31 | −0.34 | −0.74 | −1.33 |
| Acaa2 | 0.72 | 0.83 | −0.04 | −1.21 |
| Acad11 | −0.38 | −0.45 | −0.86 | −1.56 |
| Acadsb | −1.73 | −1.8 | −1.48 | −1.3 |
| Ace2 | −1.97 | −1.49 | −1.36 | −3.15 |
| Acly | −0.03 | −0.05 | −0.4 | −1.29 |
| Acot1 /// Acot2 | −0.17 | 0.26 | −0.28 | −1.24 |
| Acot2 | −1.06 | −1.04 | −0.79 | −0.33 |
| Acox1 | −0.57 | −0.62 | −0.75 | −1.45 |
| Acpp | −1.37 | −1.21 | −1.13 | −1.79 |
| Acta2 | −2.4 | −1.3 | −1.46 | −0.39 |
| Actg2 | −2.08 | −0.52 | −1.69 | −0.93 |
| Actn1 | −0.01 | 0.24 | 0.38 | 1.32 |
| Ada | 0 | 0.7 | 1.36 | 1.74 |
| Adam10 | 0.94 | 1.37 | −0.34 | −1.77 |
| Adam17 | 0.26 | 0.69 | 0 | −1.8 |
| Adam28 | −0.59 | −0.08 | −0.54 | −2.06 |
| Adamdec1 | −1.73 | −1.19 | −1.1 | −1.68 |
| Adar | −0.13 | 0 | −0.02 | −1.71 |
| Adh7 | −0.34 | 0 | 0.38 | −1.52 |
| Adi1 | −0.92 | −0.44 | −0.71 | −1.34 |
| Adk | −0.17 | 0.2 | 1.41 | 1.69 |
| Adm | 1.5 | 0.72 | −0.29 | 1.42 |
| Adssl1 | −1.47 | −1.02 | −1.3 | −1.42 |
| Aebp1 | −1.73 | −1.78 | −3.38 | 0 |
| Agpat1 | −0.34 | −0.1 | −1.25 | −1.47 |
| Agps | 0.16 | 0.38 | 1.04 | 1.25 |
| Agtrl1 | 0.07 | 0.01 | 0.62 | 2.04 |
| Ahcy | 0.89 | 1.01 | 0.84 | 0.81 |
| Ahcyl1 | 0.72 | 1.42 | −0.05 | −1.51 |
| Ahnak | −1 | −1.28 | −1.78 | −1.02 |
| Ahr | −0.47 | −0.56 | −0.58 | −1 |
| Ak3 | 0.06 | 0.13 | −0.39 | −1.35 |
| Ak3l1 | 1.17 | 0.92 | −0.56 | 1.66 |
| Akap10 | −0.12 | −0.02 | −0.3 | −2.6 |
| Akap12 | −1.72 | −1.2 | −0.39 | 0 |
| Akp2 | −0.34 | −0.22 | −1.37 | −2.43 |
| Akr1b8 | 1.06 | 0.85 | 0.24 | 0.35 |
| Aldh1a7 | 0.16 | 0.14 | −0.8 | −1.21 |
| Aldh1l1 | −1.06 | −1.39 | −1.58 | −1.93 |
| Aldh2 | −1.37 | −1.12 | −1.06 | −0.09 |
| Aldh3a1 | 0 | −0.44 | 0.5 | −1.49 |
| Aldh6a1 | −0.74 | −0.82 | −1.23 | −1.75 |
| Aldoc | 0.3 | 0.62 | 0.58 | 1.12 |
| Alg1 | −1.18 | −0.75 | −0.67 | −0.48 |
| Alg6 | 1.19 | 1.22 | 1.4 | 0.47 |
| Als2cr4 | 0.21 | 0.3 | 1.02 | 1.3 |
| Amd1 /// Amd2 | 0 | 0.04 | −0.48 | −1.86 |
| Ammecr1 | 0.52 | 0.79 | 1.14 | 1.25 |
| Amn | −1.79 | −1.94 | −1.24 | −1.25 |
| Amot | 1.03 | 1.26 | 3.38 | 3.43 |
| Ampd2 | 0.01 | 0.4 | 0.5 | 1.45 |
| Ang1 | −0.02 | 0.5 | −0.66 | −1.47 |
| Ang3 | 0.25 | 1.41 | 2.52 | 2.34 |
| Ank | 0.71 | 1.05 | 0.38 | 0.55 |
| Ankrd22 | 0.29 | 0.32 | −0.34 | −1.11 |
| Ankrd32 | 0.88 | 1.18 | 1.29 | 0.78 |
| Ankrd35 | −0.74 | −0.71 | −1.06 | −1.51 |
| Ankrd37 | 2.69 | 1.54 | 1.15 | 3.31 |
| Ankrd43 | −0.08 | 0.17 | −0.1 | −1.21 |
| Ankrd46 | −0.55 | −0.24 | −0.5 | −1.09 |
| Anln | 3.76 | 2.5 | 4.82 | 2.27 |
| Anp32b | 0.83 | 0.4 | 0.88 | 1.26 |
| Anp32e | 0.62 | 0.73 | 0.61 | 1.08 |
| Antxr1 | −1.53 | −1.63 | −1.4 | 0.16 |
| Anubl1 | 1.62 | 2.63 | 1.21 | 1.84 |
| Anxa3 | 1.66 | 1.43 | 1.41 | 1.53 |
| Anxa6 | −0.7 | −0.77 | −1.14 | −0.28 |
| Anxa8 | 0.81 | 1.28 | 0.22 | −0.46 |
| Anxa9 | 0.3 | 0.11 | 1.02 | 0.72 |
| Aof1 | −0.98 | −0.6 | −0.89 | −1.35 |
| Aox1 | −2.14 | −1.98 | −2.5 | −2.52 |
| Ap3b1 | 0.49 | 0.62 | −0.26 | −1.44 |
| Ap3m1 | 0.63 | 0.85 | 0.13 | −2 |
| Ap3m1 /// | 0.31 | 0.48 | 0.09 | −1.16 |
| LOC671800 | | | | |
| Ap3m2 | −1.36 | −1.52 | −0.92 | −0.61 |
| Apbb2 | −0.51 | −0.95 | −0.64 | −1.02 |
| Apcdd1 | −0.97 | −0.89 | −1.32 | −1.52 |
| Api5 | 0.82 | 1.22 | 0.2 | −0.31 |
| Aplp2 | −0.01 | −0.03 | −0.74 | −1.52 |
| Apoc1 | −0.04 | 0.02 | −0.61 | −1.19 |
| App | −0.32 | −0.3 | −0.81 | −1.41 |
| Aqp1 | 0.4 | −0.3 | 0.31 | 1.11 |
| Aqp3 | 0.21 | 0.39 | −0.33 | −1.44 |
| Araf | −0.08 | 0.4 | −0.47 | −1.27 |
| Arcn1 | 0.55 | 1.04 | 0.03 | −1.28 |
| Areg | 2.19 | 2.02 | 0 | 0 |
| Arf1 | 0.31 | 0.51 | −0.22 | −1.03 |
| Arf2 | 0.62 | 1.05 | 0.28 | −0.9 |
| Arfip2 | −1.04 | −1.19 | −1.05 | −1.18 |
| Arhgap1 | 0.23 | 0.34 | −0.46 | −1.05 |
| Arhgap11a | 3.2 | 3.08 | 3.31 | 2.8 |
| Arhgap26 | −1.46 | −1.63 | −1.21 | −1.43 |
| Arl14 | 0.54 | 0.64 | 1.62 | 2.07 |
| Arl2 | −0.38 | −0.82 | −0.97 | −1.01 |
| Arl5b | 0.19 | 0.91 | −0.06 | 1.12 |
| Arl6 | 0.07 | 0.56 | 0.77 | 1.16 |
| Arl6ip2 | 1.12 | 1.2 | 1.5 | 1.47 |
| Arl6ip6 /// | 0.62 | 0.95 | 0.67 | 1.26 |
| LOC674449 | | | | |
| Armcx1 | −0.7 | −0.64 | −0.98 | −2 |
| Armcx3 | −0.07 | −0.22 | −0.47 | −1.19 |
| Arrb2 | 0.69 | 0.72 | 1.01 | 1.22 |
| Arrdc3 | 0.77 | 0.92 | 0.66 | 1.17 |
| Arsa | −1.48 | −1.32 | −1.32 | −1.3 |
| Asah2 | −0.9 | −1.05 | −1.07 | −1.1 |
| Asah3l | −0.97 | −0.98 | −0.78 | −1.22 |
| Asb1 | −1.25 | −2.35 | −1.34 | −1.15 |
| Asb8 | −0.06 | 0.05 | −0.58 | −1.14 |
| Ascc2 | −1.07 | −1.1 | −1.13 | −1.61 |
| Asf1b | 2.79 | 4.82 | 2.82 | 2.6 |
| Aspm | 3.65 | 2.84 | 3.51 | 2.34 |
| Asrgl1 | 0.41 | 1.01 | 1.34 | 1.5 |
| Ass1 | −0.51 | −0.57 | −1.23 | −1.33 |
| Asxl1 | 0.59 | 0.76 | 0.75 | 1.59 |
| Atad2 | 2.26 | 2.16 | 2.32 | 1.95 |
| Atf3 | 1.83 | 2.57 | 1.51 | 1.81 |
| Atf4 | 0.36 | 0.92 | 0.57 | 1.16 |
| Atf5 | 0.83 | 0.48 | 0.59 | 1.1 |
| Atp1b1 | 0.98 | 1.53 | 1.44 | 0.87 |
| Atp2a2 | −0.44 | −0.04 | −0.56 | −1.32 |
| Atp5s | −0.5 | −0.29 | −0.56 | −1.16 |
| Atp6v0a1 | −0.77 | −1.04 | −0.59 | −0.78 |
| Atp6v1e1 | 0.28 | −1.11 | 0.43 | 0.59 |
| Atrn | −0.65 | −0.6 | −1 | −1.49 |
| Atrx | −0.48 | 0 | −0.5 | −1.59 |
| Aurka | 2.34 | 2.38 | 2.48 | 2.2 |
| Aurkb | 3.04 | 6.33 | 2.78 | 2.32 |
| AV009015 | −0.71 | −0.74 | −0.93 | −1.33 |
| Aytl2 | 0.26 | 0.2 | 0.57 | 1.04 |
| B3galnt1 | 1.12 | 1.46 | 1.23 | 3.61 |
| Bace1 | −0.85 | −0.93 | −0.78 | −1.22 |
| Bace2 | −1.14 | −1.07 | −1.21 | −0.65 |
| Baiap3 | −0.94 | −1.47 | −1.18 | −1.38 |
| Bak1 | 0.49 | 0.11 | 0.89 | 1.24 |
| Bat4 | 0.13 | 0.34 | 1.07 | 0.37 |
| Baz1a | 0.9 | 0.89 | 1.09 | 0.59 |
| Baz1b | 0.97 | 1.71 | 0.52 | 0 |
| Baz2a | −0.46 | −1.14 | −0.24 | 0.32 |
| Baz2b | 0.36 | 0 | 0.39 | −1.21 |
| Bbox1 | 0.54 | 1.77 | 0.72 | 1.17 |
| Bcam | −0.65 | −1.01 | −1.42 | −1.73 |
| Bcas1 | −2.81 | −2.4 | −1.62 | −1.73 |
| Bcl10 | 0.57 | 1.11 | 0.19 | −0.5 |
| Bcl3 | 0.53 | 1.2 | −0.13 | 1.6 |
| Bcor | 0.23 | 0.39 | 0.47 | 1 |
| Bex1 | 1.05 | 1.22 | 1.05 | 2.57 |
| Bex4 | −0.22 | −0.5 | −1.18 | −0.21 |
| Bfar | −0.57 | −0.98 | −0.32 | −1.17 |
| Bglap-rs1 /// | −1.17 | −0.73 | 1.77 | 0.69 |
| Bglap1 /// Bglap2 | | | | |
| Bgn | −1.91 | −1.54 | −1.46 | −0.59 |
| Bhlhb2 | 0.38 | 0.71 | 0.24 | 1.35 |
| Bhmt2 | −2.24 | −4.75 | −3.01 | −3.95 |
| Birc5 | 4.44 | 6.28 | 4.38 | 2.57 |
| Blm | 1.95 | 1.79 | 1.46 | 2.25 |
| Bmp2 | −2.59 | −2.66 | −2.45 | −2.37 |
| Bmp7 | −1.01 | −0.98 | −1.3 | −0.83 |
| Bms1l | 0.08 | 0.06 | 0.61 | 1.27 |
| Bnip3 | 1.52 | 1.14 | −0.12 | 1.57 |
| Bok | 1.79 | 1.72 | 1.03 | 2.18 |
| Bphl | −0.87 | −0.82 | −0.91 | −1.1 |
| Braf | −0.3 | −1.14 | −0.43 | −1.13 |
| Brca1 | 2.84 | 4.19 | 2.32 | 0 |
| Brca2 | 1.27 | 0.94 | 1.64 | 1.73 |
| Bre | 0.15 | 0.31 | −0.75 | −1.78 |
| Brrn1 | 1.77 | 1.84 | 1.82 | 2.4 |
| Brwd1 | −0.38 | −0.3 | −0.33 | −1.14 |
| Bst2 | 0.07 | 0.38 | −0.19 | 1.43 |
| Btbd11 | 0.07 | −0.27 | −0.77 | −1.33 |
| Bub1 | 5.07 | 3.52 | 3.76 | 3.16 |
| Bub1b | 5.38 | 3.54 | 5.44 | 2.81 |
| Bxdc5 | 0.49 | 1.18 | 0.23 | −0.37 |
| Bzw1 | 1.25 | 1.31 | 0.51 | −0.91 |
| C1galt1 | 0.48 | 1.17 | −0.49 | −1.67 |
| Cabc1 | −0.82 | −1 | −1.08 | −1.62 |
| Cacna1h | −2.74 | −2.49 | −1.74 | −1.85 |
| Cald1 | −2.5 | −1.35 | −1.28 | 0.67 |
| Calml3 | 0 | 0 | 4.33 | 0 |
| Camk2b | 0 | 0.03 | 1.38 | 2.66 |
| Camk2d | −1.48 | −1.77 | −0.84 | −0.94 |
| Camk2n1 | −1.48 | −1.27 | −0.82 | −0.34 |
| Cap1 | 0.46 | 0.91 | 0.24 | −1.06 |
| Capn1 | −0.07 | −0.06 | −0.86 | −1.37 |
| Capn2 | 0.84 | 0.65 | 0.71 | 1.27 |
| Car13 | 1.2 | 1.14 | 1.1 | 0.91 |
| Car2 | 0.08 | 0.73 | 1.18 | 1.23 |
| Car3 | −2.66 | −2.75 | −2.06 | −3.43 |
| Car8 /// | −2.62 | 0 | −2.42 | −1.79 |
| LOC676792 | | | | |
| Car9 | 0 | 0 | 0 | 2.22 |
| Card14 | −0.75 | −1.02 | −0.84 | −1.42 |
| Carhsp1 | 1.85 | 1.52 | 0.66 | 0.65 |
| Casc4 | −1.19 | −1.23 | −0.88 | 0.07 |
| Cask | 0.55 | 1.2 | 0.14 | −1.99 |
| Casp2 | 0.95 | 1.22 | 0.86 | 0.64 |
| Casp3 | 1.34 | 1.06 | 0.59 | 0 |
| Casp7 | 0.98 | 1.39 | 0.88 | −0.06 |
| Casp8ap2 | 0.86 | 1.35 | 1.2 | 1.53 |
| Cat | −0.62 | −0.32 | −0.78 | −1.35 |
| Cav1 | −1.27 | −0.71 | −1.81 | −0.33 |
| Cbr2 | −0.59 | −0.33 | −0.8 | −1.62 |
| Cbx2 | 0.71 | 0.73 | 0.8 | 1.63 |
| Cbx5 | 1.09 | 1.75 | 1.29 | 0.9 |
| Ccbl1 | −0.32 | −0.52 | −0.71 | −1.35 |
| Ccdc28b | 0.05 | 0.37 | 0.8 | 1.07 |
| Ccdc39 | 0.11 | −0.99 | −0.15 | −1.6 |
| Ccdc39 /// Ttc14 | −0.08 | −1.03 | 0.47 | −1.78 |
| Ccdc5 | 1.42 | 1.29 | 1.38 | 1.34 |
| Ccdc69 | −0.8 | −0.57 | −0.8 | −1.49 |
| Ccdc80 | −1.48 | −0.88 | −1.27 | −0.78 |
| Ccdc99 | 2 | 1.68 | 1.82 | 1.84 |
| Cchcr1 | 0.71 | 0.76 | 1.08 | 1.19 |
| Ccna2 | 4.56 | 3.36 | 4.72 | 2.72 |
| Ccnb1 | 5.84 | 3.32 | 5.67 | 1.98 |
| Ccnb2 | 4 | 3.17 | 4.72 | 2.18 |
| Ccnd1 | −1.92 | −2.66 | −1.38 | −1.68 |
| Ccnd2 | −4.45 | −4.1 | −2.45 | −2.86 |
| Ccne1 | 2.46 | 2.2 | 2.73 | 3.39 |
| Ccne2 | 3.33 | 4.58 | 2.65 | 3.9 |
| Ccnf | 3.26 | 2.66 | 2.89 | 2.4 |
| Ccng1 | −0.12 | 0.07 | −1.04 | −1.09 |
| Ccng2 | 0.88 | 1.34 | 0.02 | −0.48 |
| Ccnjl | −0.44 | −0.59 | −0.97 | −1.11 |
| Ccnl2 | 0.17 | 0.56 | 0.09 | −1.18 |
| Ccnt2 | 0.06 | −0.63 | −0.06 | −2.33 |
| Ccpg1 | −1.59 | −1.68 | −0.55 | −1.03 |
| Ccrn4l | 1.46 | 1.48 | 0.77 | 1.32 |
| Cd200r3 | −0.97 | 0 | 0 | −2.88 |
| Cd44 | 1.39 | 2.08 | 0.01 | −0.35 |
| Cd55 | 0.13 | 0.32 | −0.59 | −1.07 |
| Cd59a | −1.01 | −1.63 | −1.73 | −2.71 |
| Cd99l2 | −0.14 | −0.24 | −0.49 | −1.09 |
| Cdadc1 | −0.75 | −0.93 | −0.39 | −1.34 |
| Cdc20 | 2.97 | 3 | 3.65 | 2.36 |
| Cdc23 | 0.28 | 0.71 | −0.14 | −1.06 |
| Cdc25b | 1.28 | 1.94 | 1.97 | 1.91 |
| Cdc2a | 4.39 | 3.54 | 4.63 | 2.83 |
| Cdc37l1 | −0.19 | −0.78 | −0.25 | −1.62 |
| Cdc42ep2 | −1.17 | −1.04 | −0.87 | −1.84 |
| Cdc42ep3 | −0.52 | −0.37 | −1.09 | −0.84 |
| Cdc45l | 2.9 | 4.44 | 2.67 | 2.06 |
| Cdc6 | 2.81 | 3.1 | 2.38 | 2.6 |
| Cdc7 | 4.58 | 5.04 | 3.26 | 1.76 |
| Cdc73 | −0.59 | −1.09 | −0.6 | −0.72 |
| Cdca1 | 4.75 | 4.26 | 4.07 | 1.65 |
| Cdca3 | 3.02 | 2.74 | 3.24 | 2.48 |
| Cdca4 | 1.08 | 1.08 | 0.76 | 1.26 |
| Cdca5 | 2.5 | 3.1 | 4.3 | 2.05 |
| Cdca7 | 1.1 | 1.6 | 0.89 | 1.37 |
| Cdca7l | 0.95 | 0.69 | 1.06 | 1.05 |
| Cdca8 | 2.43 | 2.64 | 3.72 | 2.75 |
| Cdk2 | 1.73 | 1.68 | 1.73 | 1.29 |
| Cdk5rap2 | 0.78 | 1.12 | 2.07 | 0.83 |
| Cdk6 | −1.37 | −1.27 | −0.24 | −0.98 |
| Cdkn1a | 0.74 | 1.13 | 1.81 | 2.24 |
| Cdkn1b | 0.39 | 0.54 | 0.81 | 1.13 |
| Cdkn1c | 1.07 | 0.18 | 0 | 1.94 |
| Cdkn2a | 0 | 4.03 | 2.97 | 2.58 |
| Cdkn2c | 2.05 | 2.2 | 2.9 | 2.5 |
| Cdkn2d | 1.01 | 1.59 | 1.45 | 1.27 |
| Cdkn3 | 2.58 | 2.49 | 2.54 | 2.36 |
| Cdo1 | 1 | 0.54 | 0.85 | 1.03 |
| Cdt1 | 2.27 | 2.41 | 1.77 | 2.76 |
| Ceacam1 | 0 | 0 | 1.42 | 2.61 |
| Ceacam1 /// | −1.07 | −0.23 | 1.5 | 1.46 |
| Ceacam2 | | | | |
| Cenpa | 3.58 | 2.98 | 3.33 | 2.59 |
| Cenpe | 3.25 | 2.39 | 3.51 | 2.3 |
| Cenpf | 5.21 | 2.45 | 5.8 | 2.29 |
| Cenph | 2.89 | 3.13 | 3.34 | 1.95 |
| Cenpj | 1.26 | 1.42 | 1.58 | 1.53 |
| Centg2 | 0.27 | 0.61 | 1.17 | 1.93 |
| Cep55 | 3.81 | 3.67 | 3.51 | 2.54 |
| Cept1 | 0.67 | 0 | 0.05 | −2.06 |
| Cfd | 0 | 0 | −4 | 0 |
| Cggbp1 | 1.05 | 0.76 | 0.3 | 0.75 |
| Cgnl1 /// | −1.02 | −1.65 | −0.41 | −0.62 |
| LOC677485 | | | | |
| Chac1 | 0.61 | 0.95 | 0.45 | 1.35 |
| Chaf1b | 2.46 | 2.93 | 2.26 | 2.01 |
| Chek1 | 2.63 | 5.52 | 2.89 | 2.67 |
| Chek2 | 0.78 | 1.15 | 1.18 | 0.75 |
| Chka | 0.91 | 0.81 | 1.09 | 1.1 |
| Chst2 | −1.33 | −2.16 | −1.15 | −1.51 |
| Chtf18 | 2.29 | 1.68 | 2.51 | 1.86 |
| Chuk | 0.36 | 0.61 | −0.05 | −1.25 |
| Cirbp | −0.43 | −0.67 | −0.94 | −1.02 |
| Cited2 | 1.06 | 0.01 | −0.2 | 0.86 |
| Ckap2 | 2.97 | 2.66 | 3.06 | 2.05 |
| Ckap2l | 2.87 | 4.69 | 2.31 | 1.47 |
| Ckap4 | 0.48 | 0.16 | 0.52 | 1.08 |
| Ckap5 | 0.9 | 1.25 | 0.82 | 0.23 |
| Ckb | −2.2 | −0.95 | −1.13 | 0 |
| Cklf | 0.42 | 0.31 | 0.72 | 1.46 |
| Ckmt1 | 0.58 | 0.61 | −0.41 | −1.85 |
| Cks1b | 1.67 | 1.48 | 2.05 | 1.95 |
| Cks2 | 1.69 | 1.45 | 1.7 | 1.86 |
| Clca1 | −1.57 | −1.14 | −1.36 | −0.87 |
| Clca1 /// Clca2 | −0.94 | −0.71 | −0.98 | −1.93 |
| Clca2 | −1 | −0.7 | −0.98 | −1.32 |
| Clcn5 | −0.49 | −1.11 | −0.83 | −0.19 |
| Cldn1 | 1.25 | 2.56 | 0.84 | 0.85 |
| Cldn3 | 2.58 | 4.09 | 5.52 | 3.76 |
| Cldn8 | −0.92 | −1.03 | −0.92 | −1.48 |
| Clic4 | −0.2 | 0.41 | 0.53 | 1.05 |
| Clock /// | −0.18 | −0.44 | −0.1 | −1.23 |
| LOC620729 | | | | |
| Clspn | 3.14 | 2.91 | 4.04 | 3.21 |
| Cltc | 0.19 | 0 | −0.42 | −1.62 |
| Clu | −0.62 | −1.12 | −0.77 | −0.28 |
| Clybl | −0.75 | 0.12 | −0.66 | −1.03 |
| Cmkor1 | −0.13 | −1.43 | −0.91 | −0.5 |
| Cnn1 | −3.52 | 0 | −2.06 | 0 |
| Cnn2 | 0.1 | −0.21 | 0.34 | 1.37 |
| Cnnm2 | −1.19 | −1.23 | −0.68 | −0.93 |
| Cnnm4 | 0.28 | 0.54 | 0.1 | −1.4 |
| Cntf /// Zfp91 | 0.56 | 0.48 | 0.68 | 1.26 |
| Cntnap2 | −1.19 | −1.16 | −1.68 | −1.34 |
| Col18a1 | −0.44 | −0.46 | −1.28 | −0.87 |
| Col1a1 | −3.51 | −2.26 | −1.5 | −2.81 |
| Col1a2 | −3.82 | −2.29 | −1.63 | −1.35 |
| Col3a1 | −2.25 | −1.87 | −1.66 | −1.7 |
| Col4a1 | 0.01 | 0.45 | −0.01 | 2.37 |
| Col5a1 | −2.24 | −1.14 | −0.61 | 0 |
| Col5a2 | −2.84 | −1.55 | −1.71 | 0 |
| Col6a1 | −2.7 | −1.85 | −1.11 | −1.04 |
| Col6a2 | −2.96 | −1.93 | −1.66 | 0 |
| Col6a3 /// | −1.96 | −1.37 | −0.4 | −0.1 |
| LOC674521 | | | | |
| Copg2 | −0.36 | −0.47 | −0.61 | −1.07 |
| Copz2 | −1.43 | −1.13 | −1.64 | −1.62 |
| Coq5 | −0.04 | 0.22 | −0.36 | −1.04 |
| Coro1c | 0.64 | 1.34 | −0.42 | −0.99 |
| Coro2a | 0.65 | 1.41 | 0.9 | 1.46 |
| Cotl1 | −0.41 | −1 | −0.65 | 0.18 |
| Cpd | 0.87 | 0.96 | 0.01 | −1.68 |
| Cpeb2 | 0.98 | 1.29 | 0.45 | −0.75 |
| Cpsf2 | 0.64 | 1.29 | 0.8 | −0.28 |
| Cpsf3l | 0.82 | 0.71 | 1.23 | 0.8 |
| Crabp2 | 0.39 | −1.02 | −1.72 | −1.23 |
| Creb3l2 | −1.14 | −1.37 | −1.33 | −1.3 |
| Crebl2 | −1.21 | −1.05 | −0.93 | −0.84 |
| Creld1 | −0.6 | −0.56 | −0.98 | −1.17 |
| Cri1 | −0.33 | −0.18 | −0.21 | 1.01 |
| Crim1 | −0.56 | −1.4 | −1.04 | −1.03 |
| Crip1 | 0.51 | 0.79 | 0.45 | 1.71 |
| Crispld2 | −3.85 | −2.68 | −2.65 | 0 |
| Crot | −0.2 | −0.14 | −0.42 | −1.01 |
| Cry1 | 0.39 | 0.93 | 0.7 | 1.1 |
| Cryz | −0.84 | −1.15 | −0.88 | −0.34 |
| Cryzl1 | 0.07 | 0.45 | −0.49 | −1.25 |
| Csad | −0.86 | −0.76 | −1.08 | −0.96 |
| Csf1r | −1.18 | −0.92 | −0.28 | 1 |
| Csnk1a1 | 0.05 | −0.77 | −0.2 | −1.74 |
| Csnk1e | −0.06 | 0.25 | 0.3 | 1 |
| Cspg5 | −1.66 | −1.13 | −1.37 | −2.1 |
| Csprs /// | 0 | 0 | 2.04 | 3.11 |
| LOC665338 | | | | |
| Csrp1 | 0.27 | 1.08 | 0.63 | 0.82 |
| Cstf2 | 1.09 | 1.23 | 1.22 | 0.39 |
| Ctf1 | −0.6 | −0.71 | −0.61 | −1.11 |
| Ctla2a | 0.6 | −0.33 | 0 | 1.73 |
| Ctnnal1 | −0.15 | −0.28 | −1.39 | −2.27 |
| Ctnnb1 | 0.84 | 1.26 | −0.03 | −1.98 |
| Ctnnd2 | −1.06 | −0.85 | −1.72 | −1.3 |
| Ctns | −1.2 | −1.6 | −1.04 | −1.69 |
| Ctsd | −0.68 | −0.98 | −1.1 | −1.05 |
| Ctse | 2.69 | 2.5 | 1.87 | 0 |
| Ctss | −0.49 | 0.94 | 0.72 | 1.59 |
| Cttn | −0.02 | 0.53 | −0.38 | −1.7 |
| Cxadr | 1.52 | 1.15 | 1.54 | 0.34 |
| Cxcl10 | 3.99 | 2.97 | 0 | 3.34 |
| Cxcl14 | −3.03 | −1.82 | −1.12 | −1.38 |
| Cxcl16 | 0.97 | 1.42 | 1 | 0.85 |
| Cxx1b | −0.37 | −0.8 | −1.05 | −1.07 |
| Cyb5b | −0.23 | 0 | −0.94 | −1.47 |
| Cyb5r3 | 0.03 | 0.08 | −0.94 | −3.06 |
| Cyba | −0.26 | 0.29 | 0.49 | 1.5 |
| Cyfip2 | −1 | −1.1 | −1.04 | −1.76 |
| Cyhr1 | −1.17 | −1.08 | −0.83 | −1.12 |
| Cyp1a1 | −2.52 | −2.16 | −1.12 | −3.01 |
| Cyp1b1 | −1.39 | −1.05 | −0.36 | −1.96 |
| Cyp27a1 | −0.19 | −0.04 | 0.38 | −1.56 |
| Cyp2f2 | −2.71 | −0.25 | −0.82 | −1.75 |
| Cyp4f15 | −2.27 | −2.13 | −2.01 | −3.15 |
| Cyp4v3 | −1.23 | −0.95 | −0.7 | −0.44 |
| Dab1 | −1.48 | −0.47 | −0.42 | −1.63 |
| Dab2 | −1.58 | −1.76 | −2.62 | −1.89 |
| Dalrd3 | −0.32 | −0.21 | −0.66 | −1.08 |
| Dapk1 | −0.23 | −0.44 | −1.14 | −1.43 |
| Dapp1 | −1.57 | −1.43 | −0.37 | −0.68 |
| Dbf4 | 1.79 | 1.69 | 2.05 | 1.77 |
| Dbp | −1.63 | −2.04 | −1.36 | −1.36 |
| Dcbld2 | 0.86 | 0.72 | 1.59 | 0.99 |
| Dck | 1.77 | 2.08 | 2.39 | 2.48 |
| Dcn | −1.96 | −1.92 | −1.87 | −1.87 |
| Dctd | 0.65 | 1.19 | 0.46 | 0.24 |
| Dcun1d1 | 0.65 | 1.09 | 0.11 | −0.62 |
| Dcxr | −0.77 | −0.5 | −1.11 | −0.81 |
| Ddx11 | 1.13 | 3.59 | 1.05 | 0.93 |
| Ddx20 | 1.02 | 0.62 | 0.8 | 0.45 |
| Ddx39 | 1.24 | 1.11 | 1.18 | 1.8 |
| Ddx3y | −4.12 | −0.44 | 0 | 3.54 |
| Ddx46 | 1.21 | 1.29 | 0.54 | −0.77 |
| Decr1 | −0.75 | −0.62 | −0.52 | −1.16 |
| Decr2 | −0.71 | −0.32 | −0.77 | −1.31 |
| Defb1 | −2.27 | −2.49 | −2.12 | −3.18 |
| Degs1 | −1.08 | −0.17 | 0.56 | 1.7 |
| Dek | 0.84 | 1.01 | 1.03 | 1.04 |
| Dennd2d | −0.37 | −0.71 | −0.77 | −1.05 |
| Depdc1a | 3.33 | 2.94 | 2.97 | 1.95 |
| Depdc6 | −1.13 | −0.89 | −0.65 | −2.12 |
| Dfna5h | −0.03 | 0.37 | −0.82 | −1.45 |
| Dgcr6 | −1.42 | −1.59 | −1.13 | −0.98 |
| Dgkd | 0.08 | 0.33 | 0.36 | 1.17 |
| Dhfr | 2.09 | 2.37 | 2.09 | 2.15 |
| Dhrs3 | −0.96 | −0.9 | −0.54 | −1.08 |
| Diap1 | −0.25 | 0 | −0.04 | −1.76 |
| Diap3 | 3.8 | 2.34 | 4.13 | 2.57 |
| Dlc1 | −0.7 | −0.73 | −0.49 | −1.03 |
| Dleu8 | 1.1 | 0.73 | 1.22 | 1.09 |
| Dlg7 | 2.62 | 2.68 | 2.47 | 1.96 |
| Dmgdh | −2.23 | −2.77 | −4.15 | −4.42 |
| Dmn | −3.14 | 0 | −1.04 | 0 |
| Dna2l | 2 | 2.22 | 2.6 | 2.15 |
| Dnaja2 | −0.1 | −0.75 | −0.29 | −1.43 |
| Dnaja3 | 0.58 | 0.64 | 0.06 | −1.3 |
| Dnaja4 | 0.02 | 0.36 | −0.56 | −1.14 |
| Dnajb1 | 0.63 | 1.09 | −0.06 | 0 |
| Dnajb14 | −0.94 | −1.07 | −0.6 | 0.51 |
| Dnajc1 | 0.5 | 0 | −0.15 | −2.07 |
| Dnajc12 | −0.53 | −0.55 | −0.2 | −1.06 |
| Dnajc3 | −0.14 | 0 | −0.25 | −1.02 |
| Dnajc5 | 1 | 0.73 | 0.12 | 0.21 |
| Dnajc6 | −1.32 | −1.71 | 0.1 | −0.49 |
| Dnajc9 | 1.57 | 1.68 | 1.82 | 1.41 |
| Dnm1l | 0.35 | 0.49 | −0.32 | −1.07 |
| Dnm2 | 0.47 | 0.65 | −0.4 | −2.08 |
| Dnmt1 | 1.37 | 1.33 | 1.8 | 2.06 |
| Dnmt3l | −0.31 | −0.48 | −1.02 | −1.57 |
| Dock11 | 1.05 | 2.22 | 2.72 | 2.96 |
| Dock8 | −0.68 | −0.63 | −0.81 | −1.04 |
| Dock9 /// | −0.51 | −0.63 | −0.79 | −1.56 |
| LOC670309 | | | | |
| Donson | 1.53 | 1.09 | 1.65 | 1.65 |
| Dpt | −2.47 | −1.52 | −1.26 | 0 |
| Dpysl2 | 1.03 | 1.56 | 2.25 | 2.84 |
| Dr1 | 0.68 | 1.12 | −0.07 | −0.68 |
| Dsc2 | 0.14 | 1.41 | 0.66 | −0.81 |
| Dscr1 | −0.21 | 0.69 | −1.34 | −2.79 |
| Dsg2 | −0.19 | −0.5 | −0.57 | −1.13 |
| Dsp | 1.24 | 1.53 | 0.45 | −1.72 |
| Dst | −0.91 | −1.52 | −0.48 | −0.84 |
| Dtl | 4.38 | 3.86 | 2.84 | 3.29 |
| Dtnb | 0.38 | 1.4 | 0.53 | 0.13 |
| Dusp1 | 1.17 | 0.95 | 0.66 | 1.55 |
| Dusp16 | −0.02 | 0 | −0.28 | −1.56 |
| Dusp9 | 1.2 | 1.01 | −0.17 | 0.35 |
| Dut | 1.6 | 2.02 | 1.86 | 2.03 |
| Dvl1 | −0.18 | −0.16 | 0.58 | 1.27 |
| Dynll1 /// | 0.33 | 0.42 | 0.8 | 1.09 |
| LOC627788 /// | | | | |
| LOC637840 /// | | | | |
| LOC672375 | | | | |
| Dynlt1 /// | 1.04 | 1 | 0.88 | 0.45 |
| LOC671261 | | | | |
| Dynlt3 | 0.14 | 0.37 | −0.87 | −1.88 |
| Dyrk1a | 0.8 | 1.18 | 0.22 | −1.08 |
| Dysf /// Fer1l3 | −0.63 | −0.56 | −0.54 | −1.42 |
| E2f1 | 1.58 | 3.19 | 2.41 | 3.4 |
| E2f7 | 3.54 | 2.27 | 3.33 | 2.69 |
| E2f8 | 5.17 | 3.29 | 3.27 | 3.98 |
| Ecgf1 | −0.56 | −0.69 | −1 | −1.05 |
| Ecm1 | −1.44 | −1.03 | −0.99 | −0.56 |
| Ect2 | 2.59 | 2.37 | 2.88 | 2.25 |
| Edg7 | −2.37 | −1.03 | −1.38 | −4.22 |
| Efemp1 | −1.85 | −1.82 | −1.61 | −0.84 |
| Egfl6 | −1.27 | −0.73 | −2.92 | −2.46 |
| Egln3 | 1.62 | 1.4 | −0.23 | 0.94 |
| Egr1 | 1.04 | 3.02 | 2.18 | 2.66 |
| Ehf | 0.24 | 0.33 | −0.16 | −1.27 |
| Eif2ak1 | 0.1 | −0.04 | 0 | −1.33 |
| Eif2ak2 | 0.73 | 1.02 | 0.71 | −0.64 |
| Eif2s3x /// | 1.01 | 1.24 | 0.03 | −0.86 |
| LOC673373 | | | | |
| Eif2s3y | −4.02 | −0.69 | 0 | 3.71 |
| Eif4a1 | 0.66 | 1.02 | 0.08 | −1.03 |
| Eif4a2 | −0.62 | −0.44 | −0.81 | −1.16 |
| Eif4ebp2 | 0.36 | 0.46 | −0.47 | −1.64 |
| Eif4g1 | 0.28 | 1.13 | −0.59 | −0.5 |
| Eif5 | −0.25 | −0.61 | −0.33 | −1.14 |
| Ela1 | −1.49 | −1.69 | −1.98 | −2.1 |
| Elavl1 | 0.05 | 0 | −0.63 | −1.78 |
| Elf2 | 0.63 | 1.17 | 0.61 | 0.44 |
| Elf3 | −0.06 | 0.41 | 0.81 | 1.01 |
| Elf5 | −1.85 | −1.89 | −1.7 | −2.46 |
| Ell2 | 0.91 | 1.09 | 1.02 | 1.22 |
| Ell3 | 0.68 | 1.09 | 0.67 | 1.21 |
| Elovl5 | 0.55 | 0.32 | 0.84 | 1.07 |
| Emd | 0.57 | 0.52 | 0.64 | 1.03 |
| Eme1 | 3.94 | 1.65 | 3.36 | 1.39 |
| Emid1 | −2.24 | −1.37 | −1.69 | −2.4 |
| Emp1 | 1.45 | 0.82 | 0.86 | 1.34 |
| Emp2 | 0.62 | 0.54 | 0.06 | −1.71 |
| Enah | −0.34 | −0.39 | −0.93 | −1.02 |
| Enc1 | −1.38 | −0.93 | −0.6 | 0.06 |
| Enpp1 | 0.45 | 0.47 | −0.57 | −1.38 |
| Enpp5 | −0.48 | −0.22 | −0.49 | −1.41 |
| Entpd3 | −0.91 | −0.83 | −1.16 | −1.62 |
| Entpd4 | −0.92 | −1.16 | −0.74 | −0.23 |
| Entpd5 | −0.37 | −0.32 | −1.38 | −1.81 |
| Epb4.1l3 | −1.47 | −2.25 | −2.54 | −3.31 |
| Epb4.1l4a | −0.72 | −1.16 | −0.61 | −0.91 |
| Epb4.9 | −0.95 | −1.31 | −1.17 | −1.26 |
| Ephb2 | −0.56 | −1.17 | 0.46 | 0.46 |
| Ephx1 | −1.54 | −1.14 | −1.02 | −1.31 |
| Ephx2 | −1.28 | −1.13 | −1.37 | −1.08 |
| Epm2aip1 | −0.43 | −0.62 | −0.57 | −1.55 |
| Eppk1 | 1.15 | 0.63 | 1.05 | 1.18 |
| Epsti1 | 0 | 0 | 1.35 | 1.44 |
| Erbb2ip | 0.15 | 0 | −0.31 | −2.06 |
| Ercc6l | 3.11 | 2.69 | 3.44 | 2.57 |
| Ern1 | −1.35 | −1.08 | −1.11 | −0.66 |
| Ero1l | 1.77 | 1.12 | 0.24 | 1.16 |
| Errfi1 | 0.94 | 1.51 | 0.9 | −0.19 |
| Esco2 | 3.24 | 3.28 | 3.35 | 2.84 |
| Espl1 | 2.43 | 2.09 | 4.14 | 1.92 |
| Etv6 | 0.03 | 0 | −0.02 | −2.19 |
| Eva1 | 0.48 | 0.88 | −0.68 | −1.1 |
| Exdl1 | −0.96 | −1.1 | −1.25 | −0.58 |
| Exo1 | 5.45 | 3.23 | 2.55 | 2.36 |
| Exoc4 | −0.03 | 0 | −0.64 | −1.71 |
| Exod1 | 0.92 | 1.02 | 0.9 | 0.12 |
| Exosc2 | 0.97 | 1.15 | 0.82 | 0.68 |
| Exosc6 | 0.6 | 0.67 | 0.7 | 1.21 |
| Exosc8 | 1.41 | 1.41 | 1.33 | 1.43 |
| Ext1 | −0.35 | 0 | −0.51 | −2.33 |
| Ezh2 | 0.98 | 1.29 | 1 | 1.67 |
| F2r | −1.69 | −1.78 | −0.47 | 0.59 |
| F2rl1 | 0.54 | 1.04 | 0.52 | 0.86 |
| F3 | 0 | 4.44 | 1.21 | 1.58 |
| Faah | −1.21 | −1.07 | 0.32 | −0.23 |
| Fabp4 | 0.14 | −0.71 | −0.69 | −1.74 |
| Fads2 | 0.15 | −0.04 | −0.93 | −1.81 |
| Faf1 | 0.91 | 1.18 | 0.48 | −0.54 |
| Falz | −0.32 | −1.32 | −0.63 | −1.54 |
| Fanca | 1.2 | 0.84 | 0.81 | 1.02 |
| Fancb | 0.76 | 0.94 | 1.09 | 0.86 |
| Fath2 | −0.6 | −1.06 | −1.05 | −1.36 |
| Fbln1 | −1.87 | −0.97 | −0.96 | 0 |
| Fbln5 | −2.63 | −1.48 | −2.06 | 0 |
| Fbp1 | 0.38 | 0.3 | −0.23 | −1.08 |
| Fbxl3 | 0.43 | 1.58 | 0.51 | −1.01 |
| Fbxo21 | −0.73 | −1.03 | −0.49 | −0.48 |
| Fbxo39 | 0.29 | 1.01 | 0.84 | 1.2 |
| Fbxo5 | 2.06 | 1.82 | 2.39 | 2.03 |
| Fbxw11 | 0.52 | 1.2 | −0.37 | −2.08 |
| Fbxw2 | 0.71 | 0.97 | 0.3 | −1.18 |
| Fbxw7 | −0.2 | −1.09 | −0.34 | 0.22 |
| Fen1 | 2.64 | 2.96 | 2.86 | 2.11 |
| Fer1l4 | −0.86 | −1.11 | −0.72 | −0.73 |
| Fetub | 1.08 | 1.44 | 0.78 | 0.63 |
| Fgd4 | −0.64 | −1.11 | −0.92 | −0.69 |
| Fgf1 | −2.02 | −1.64 | −1.78 | −2.61 |
| Fgfr2 | 0.15 | 0.21 | −0.96 | −1.57 |
| Fhl1 | −2.3 | −1.93 | −1.73 | −1.87 |
| Fignl1 | 3.52 | 3.12 | 5.22 | 2.68 |
| Fjx1 | −0.82 | −1.5 | −0.99 | 0 |
| Flnb | 0.56 | −1.04 | −0.08 | −1.47 |
| Flot2 | 0.21 | 0.38 | 0.73 | 2.31 |
| Flrt3 | −0.7 | −1.39 | −1.99 | −1.36 |
| Fmo2 | −2.34 | −1.98 | −1.03 | −2.07 |
| Fmo5 | −1.55 | −1.3 | −1.03 | −2.04 |
| Fn1 | −1.21 | −1.17 | −1.21 | 0.52 |
| Foxm1 | 0.86 | 3.46 | 1.56 | 1.72 |
| Foxm1 /// Pebp1 /// | 2.75 | 2.49 | 2.43 | 2.65 |
| 4933413G19Rik | | | | |
| Foxp2 | −0.7 | −1.1 | −1.41 | −1.23 |
| Fras1 | −0.33 | −0.67 | −1.28 | −0.44 |
| Fry | −1.05 | −1.15 | −0.86 | −0.93 |
| Fscn1 | 0.75 | 0.9 | −0.49 | −1.61 |
| Fshprh1 | 4.7 | 5.27 | 3.87 | 3 |
| Fstl1 | −2.34 | −1.77 | −1.57 | −0.33 |
| Fubp1 | 0.03 | −0.35 | 0.13 | −2.17 |
| Fusip1 | 0.9 | 1.02 | 0.64 | 0.22 |
| Fut4 | −1.11 | −0.97 | −1.25 | −2.59 |
| Fut9 | −0.8 | −1.05 | 0.03 | −0.37 |
| Fxyd4 | 2.15 | 1.14 | 0.98 | 1.87 |
| Fxyd6 | 0.99 | 1.26 | 1.75 | 2.26 |
| Fyco1 | −1.05 | −1.2 | −0.71 | −1.29 |
| Fzd2 | −1.45 | −1.36 | −1.13 | −2.36 |
| Fzd6 | 0.49 | 1.03 | −0.16 | −0.93 |
| G6pd2 /// G6pdx | 0.26 | 0.15 | −0.63 | −1.77 |
| Gabpb1 | 1.04 | 1.76 | 0.91 | 0.51 |
| Gabrp | 0 | 0.58 | 1.43 | 0.39 |
| Gadd45a | −0.89 | −0.82 | −1.09 | −0.79 |
| Gadd45b | −0.03 | 0.83 | 1.09 | 1.74 |
| Galnt10 | −0.81 | −0.98 | −0.47 | −1.08 |
| Galnt12 | 0.8 | 1.16 | 0.74 | 0.43 |
| Galnt4 | −0.67 | −0.22 | −0.38 | −1.08 |
| Galnt6 | 0.81 | 0.35 | −1.05 | −2.06 |
| Galnt7 | −0.23 | 0.34 | −0.43 | −1.37 |
| Galntl1 | −2.69 | −2.73 | −2.59 | −3.28 |
| Garnl1 | 0.65 | 0.85 | 0.32 | −1.11 |
| Garnl4 | −1.48 | −1.08 | −1.15 | −1.38 |
| Gas6 | −1.75 | −1.56 | −2.03 | −2.11 |
| Gata6 | −0.43 | −1.16 | −1.61 | −1.98 |
| Gcn5l2 | −0.17 | −0.52 | −1.07 | −1.04 |
| Gcnt1 | −0.46 | −0.32 | −1.22 | −1.21 |
| Gcnt2 | −1.99 | −2.13 | −1.94 | −1.18 |
| Gdap10 | −0.26 | −0.67 | 0.01 | −1.53 |
| Gdi2 | 0.55 | 0.66 | −0.21 | −1.82 |
| Gdpd5 | −0.91 | −0.95 | −1.12 | −0.77 |
| Gem | −0.03 | 0.3 | −0.56 | −1.03 |
| Gemin6 | 1.14 | 1.1 | 1.61 | 1.23 |
| Gfra3 | −1.67 | −1.84 | −1.26 | −2.22 |
| Gga2 | −0.61 | −0.69 | −0.95 | −1.52 |
| Gig1 | −0.61 | −1.22 | −0.92 | −0.11 |
| Gins4 | 2.21 | 1.77 | 1.94 | 1.64 |
| Gipc2 | 1.16 | 0.56 | 0.62 | 1.03 |
| Gjb6 | −0.32 | −0.22 | −0.71 | −1.19 |
| Gldc | −1.06 | −0.67 | −0.27 | −0.89 |
| Glrx | −0.3 | −0.49 | −0.42 | −1.92 |
| Gls | −0.82 | −1.39 | −0.48 | −0.85 |
| Gm1752 | −1.02 | −1.02 | −0.32 | −0.45 |
| Gm70 | −0.34 | −0.59 | −1.18 | −1.12 |
| Gm967 | −0.55 | −0.42 | −1.1 | −1.07 |
| Gmcl1 | 0.52 | 1.42 | 0.14 | −1.66 |
| Gmfb | 0.28 | 0.41 | −0.11 | −2.25 |
| Gmnn | 2.34 | 1.93 | 2.15 | 2.1 |
| Gna13 | 0.59 | 1.19 | 0.11 | 0.05 |
| Gna-rs1 | 0.03 | 0.12 | 0.55 | 1.21 |
| Gne | −1.77 | −1.98 | −1.49 | −2.22 |
| Gng10 | 0.08 | 0.13 | 0.62 | 1.04 |
| Gns | −1.38 | −1.22 | −0.81 | −0.79 |
| Golgb1 | −0.66 | −1.13 | −0.43 | −0.59 |
| Golph3 | 0.4 | 1.08 | −0.33 | −1.98 |
| Golph3l | 0.31 | 1.15 | −0.61 | −1.03 |
| Golph4 | −0.32 | −0.38 | −1.06 | −1.65 |
| Gorasp1 | −0.67 | −0.56 | −0.71 | −1.08 |
| Gorasp2 | 0.17 | 0.29 | −0.36 | −1.51 |
| Got1 | −0.27 | −0.24 | −0.82 | −1.66 |
| Got2 | 0.42 | 0.33 | −0.32 | −1.8 |
| Gpa33 | 0.37 | 0.7 | −0.52 | −2.5 |
| Gpam | 0.09 | 0.55 | −0.85 | −2.27 |
| Gpbp1 | 0.84 | 1.27 | −0.05 | −1.56 |
| Gpc4 | −1.41 | −1.29 | −1.45 | −1.69 |
| Gpiap1 | 0.88 | 1.04 | −0.38 | −1.98 |
| Gpihbp1 | 1.51 | 1.66 | 1.21 | 3.27 |
| Gpld1 | −1.09 | −1.21 | −1.25 | −1.38 |
| Gpm6a | −0.92 | 0 | −3.24 | −2 |
| Gpr109a | 1.03 | 0.94 | 0.13 | −0.35 |
| Gpr115 | 1.2 | 0.86 | 0.95 | 1.08 |
| Gpr137b | −1.02 | −0.66 | −0.43 | −0.49 |
| Gpr39 | −0.15 | −0.07 | −0.32 | −1.01 |
| Gpr64 | −0.53 | −0.94 | −1.57 | −0.94 |
| Gprc5a | 1.3 | 1.44 | 0.8 | 1.43 |
| Gprk6 | 0.32 | 0.59 | 0.6 | 1.2 |
| Gpsm2 | 1.16 | 1.32 | 1.21 | 1.48 |
| Gpt1 | −0.75 | −0.36 | −1.01 | −1.16 |
| Gpx2 | 0.85 | 0.37 | 1.5 | 0.69 |
| Gpx3 | −1.97 | −1.79 | −1.53 | −2.05 |
| Grhl2 | −0.51 | −0.7 | −0.65 | −1.16 |
| Grina | −1.06 | −1.13 | −0.71 | −0.39 |
| Gsg2 | 4.64 | 4.08 | 3.91 | 2.39 |
| Gsta3 | −0.89 | −1.28 | −1.51 | −2.49 |
| Gstm1 | −0.51 | −0.54 | −0.92 | −1.65 |
| Gstm1 /// | −1.2 | −0.91 | −1.59 | −2.2 |
| LOC433943 /// | | | | |
| LOC674086 | | | | |
| Gstm2 | −0.07 | −0.02 | −1.08 | −1.03 |
| Gstm3 | −0.88 | −0.87 | −1.39 | −2.82 |
| Gstm4 | −0.74 | −0.54 | −1.01 | −1.34 |
| Gstm5 | 0.03 | 2.62 | 0.46 | 2.16 |
| Gstt3 | −0.64 | −0.58 | −0.58 | −1.07 |
| Gtf2h4 | 0.49 | 0.19 | 0.6 | 1.08 |
| Gtf2i /// | −0.28 | −0.07 | −0.36 | −1.78 |
| LOC669007 | | | | |
| Gtl2 | −2.35 | −4.7 | −4.06 | −5.59 |
| Gtl2 /// Lphn1 | −1.29 | −3.28 | −2.07 | −4.05 |
| Gtlf3b | −0.94 | −0.97 | −1 | −0.97 |
| Gtpbp2 | 0.68 | 1.17 | 0.77 | −0.68 |
| Gtse1 | 1.34 | 1.11 | 0.8 | 1.19 |
| H19 | 4.7 | 2.52 | 1.68 | 4.19 |
| H1f0 | 1.74 | 1.95 | 1.45 | 1.22 |
| H1fx | 0.89 | 0.81 | 1.73 | 2.5 |
| H2-Aa | −1.03 | −0.62 | 0.12 | 0.41 |
| H2afv | 0.84 | 0.82 | 0.88 | 1.11 |
| H2afx | 1.61 | 1.59 | 2.02 | 2.34 |
| H2afz /// | 0.89 | 0.73 | 1.26 | 1.47 |
| LOC631864 /// | | | | |
| LOC675899 | | | | |
| H2-B1 | −1.39 | −0.91 | −1.51 | −1.63 |
| H2-K1 | −1.65 | −0.68 | 0.14 | 0.3 |
| Hadhsc | −0.16 | −1 | −0.32 | −0.28 |
| Hars | 0.21 | −0.03 | 0.38 | 1.14 |
| Hars2 | −1.06 | −0.91 | −0.61 | −0.87 |
| Has3 | 1.09 | 0.65 | −0.11 | −0.55 |
| Hat1 | 1.8 | 1.85 | 1.21 | 1.38 |
| Hbld2 | 0.3 | 0.52 | −0.35 | −1.02 |
| Hbp1 | 0.42 | 0.88 | −0.17 | −1.78 |
| Hbs1l | 1.02 | 1.34 | 0.12 | −1.04 |
| Hccs | 0.94 | 1.32 | 0.64 | −0.54 |
| Hcfc1 | 0.64 | 1.07 | 0.94 | −0.29 |
| Hck | −0.57 | −0.61 | −0.31 | −1.1 |
| Hdac11 | −1.57 | −1.45 | −1.16 | −1.6 |
| Hdgf | 0.61 | 0.48 | 1.07 | 1.4 |
| Hebp2 | 0.23 | 0.5 | 1.06 | 0.7 |
| Helb | 0.47 | 0.86 | 1.06 | 1.01 |
| Hells | 3.2 | 3.6 | 2.94 | 3.05 |
| Hemk2 | −0.85 | −1.01 | −0.59 | 0.04 |
| Herpud1 | −0.56 | −0.51 | −0.66 | −1.05 |
| Hexim1 | −0.33 | −0.06 | 0.19 | −1.05 |
| Hif1a | 1.54 | 1.61 | −0.05 | −1.75 |
| Higd1a | 1.12 | 0.7 | 0.57 | 1.64 |
| Hip1r | −0.24 | −0.16 | −0.62 | −1.61 |
| Hirip3 | 1.74 | 2.13 | 1.84 | 1.78 |
| Hist1h1c | 0.89 | 0.78 | 0.48 | 1.31 |
| Hist1h4h /// | 2.01 | 0.82 | 0.36 | 1.03 |
| Hist1h4c /// | | | | |
| Hist1h4i /// | | | | |
| Hist1h4j /// | | | | |
| Hist1h4k /// | | | | |
| Hist1h4m /// | | | | |
| Hist1h4a /// | | | | |
| Hist1h4b | | | | |
| Hist1h4i | 1.26 | 0.58 | 0.23 | 0.81 |
| Hist2h2be | 1.07 | 1.29 | 1.67 | 1.74 |
| Hist2h3c1 /// | −1.41 | −2.11 | −1.36 | −0.8 |
| Hist2h2aa1 /// | | | | |
| Hist1h2ad /// | | | | |
| Hist1h2an /// | | | | |
| Hist2h2ab /// | | | | |
| Hist2h2ac /// | | | | |
| Hist2h2aa2 | | | | |
| Hist2h3c1 /// | 1.73 | 1.2 | 0.59 | 0.45 |
| Hist2h3c2 /// | | | | |
| Hist1h3g /// | | | | |
| Hist1h3f /// | | | | |
| Hist1h3c /// | | | | |
| Hist1h3d /// | | | | |
| Hist1h3b /// | | | | |
| Hist1h3e /// | | | | |
| Hist1h3h /// | | | | |
| Hist1h3i /// | | | | |
| Hist2h3b /// | | | | |
| Hist1h3a | | | | |
| Hk2 | 1.54 | 0.58 | 0.02 | 1.25 |
| Hlcs | −0.58 | −0.6 | −0.64 | −1.04 |
| Hmg20a | −0.29 | −0.62 | −0.64 | −1.79 |
| Hmgb1 /// | 1.03 | 1.31 | 0.61 | −0.11 |
| 4932431P20Rik /// | | | | |
| LOC432959 /// | | | | |
| LOC434174 /// | | | | |
| LOC545555 /// | | | | |
| LOC637733 /// | | | | |
| LOC640060 /// | | | | |
| LOC667777 /// | | | | |
| LOC669546 | | | | |
| Hmgb2 /// | 2.46 | 2.84 | 2.72 | 1.99 |
| LOC433785 /// | | | | |
| LOC678110 /// | | | | |
| LOC678341 | | | | |
| Hmgb2 /// | 1.96 | 2.42 | 1.78 | 2.08 |
| LOC678110 /// | | | | |
| LOC678341 | | | | |
| Hmgcs2 | −0.54 | −0.85 | −1.69 | −2.16 |
| Hmgn2 | 1 | 1.46 | 1.57 | 1.52 |
| RHAMM | 3.56 | 2.97 | 3.16 | 1.76 |
| Hmox1 | 1.01 | 1 | −0.07 | 0.42 |
| Hn1 | 0.43 | 0.95 | 0.44 | 1.73 |
| Hnrpd | 0.77 | 0.42 | 0.53 | 1.17 |
| Hnrpl | 0.5 | 0.67 | 0.8 | 1.07 |
| Hnrpr | 0.88 | 1.02 | 0.5 | −0.58 |
| Hnrpu | 0.23 | −0.09 | 0.39 | −1.27 |
| Hpgd | −1.11 | −0.84 | −0.68 | −0.2 |
| Hr | −0.42 | 0.13 | 1.09 | 0.28 |
| Hrsp12 | −0.87 | −1.16 | −1.12 | −1.17 |
| Hs1bp3 | −0.4 | −1.02 | −0.19 | −0.45 |
| Hs3st1 | 0.81 | 1.02 | 0.46 | 0.7 |
| Hsd17b12 | 0.42 | 0.65 | 0 | −1.18 |
| Hsd17b2 | −0.97 | −1.45 | −1.75 | −1.79 |
| Hsp110 | 0.43 | 1.08 | −0.15 | −0.31 |
| Hspa14 | 0.85 | 1.08 | 1.09 | 1.35 |
| Hspa1a | 1.13 | 1.77 | 0.35 | 1.04 |
| Hspa4 | −0.16 | −0.29 | −0.23 | −1.43 |
| Hspa4l | 1 | 1.18 | 1.03 | 0.33 |
| Htatip2 | −0.64 | −0.87 | −1.09 | −1.06 |
| Htr4 | −0.78 | −0.85 | −1.48 | −1.49 |
| Htra1 | −3.49 | −2.4 | −1.95 | −0.46 |
| Htra2 | −0.03 | 0.17 | 0.22 | 1.05 |
| Ibrdc1 | 0.21 | 0.21 | 0.18 | 1.04 |
| Ibrdc2 | −0.23 | 0.07 | −0.89 | −1.97 |
| Ibrdc3 | 0.17 | 0.36 | 0.75 | 1.16 |
| Ica1 | 0.29 | 0.67 | −0.08 | −1.41 |
| Icosl | −0.21 | −0.24 | −0.14 | −1.11 |
| Id2 | −0.38 | −0.31 | −0.35 | −1.88 |
| Id4 | −1.5 | −1.34 | −0.76 | −3.41 |
| Ide | 0.38 | 0.89 | −0.01 | −1.78 |
| Idh1 | 0 | 0.08 | −0.35 | −1.04 |
| Ids | −1.17 | −1.01 | −0.53 | −0.49 |
| Ier3 | 0.79 | 0.9 | 0.64 | 1.01 |
| Ifi202b | 2.09 | 2.3 | 0.86 | 3.59 |
| Ifi203 | 1.97 | 3.26 | 1.62 | 2.49 |
| Ifi30 | 0.67 | 0.78 | 1.12 | 1.04 |
| Ifih1 | 0.21 | 0.8 | 0.82 | 1.4 |
| Ifit1 | 1.46 | 1.93 | 1.59 | 3.18 |
| Ifit2 | 1.66 | 2.16 | 2.1 | 2.91 |
| Ifnz | 1.59 | 0.94 | 1.5 | 1.69 |
| Ifrd1 | 0.84 | 1.08 | 0.75 | 1.03 |
| Ift57 | 0 | −0.06 | 1.05 | 0.7 |
| Igf1r | 0.18 | 0 | −0.86 | −1.97 |
| Igf2 | −0.48 | 0.06 | −1.01 | 1.56 |
| Igf2bp3 | 0.97 | 1.49 | 0 | 0 |
| Igfbp2 | −0.23 | −0.35 | −0.79 | −2.01 |
| Igfbp3 | 0.56 | 0.6 | 1.08 | 2.15 |
| Igfbp4 | −1.62 | −1.26 | −1.28 | 0.27 |
| Igfbp5 | −1.13 | −1.26 | −2.25 | −1.17 |
| Igfbp6 | −0.76 | 0 | −1.65 | −1.45 |
| Igk-V28 /// Igk-V8- | 0 | −1.12 | 0 | 1.86 |
| 16 | | | | |
| Igsf4a | 0.27 | −0.1 | 0.62 | 1.06 |
| Igsf4c | 1.97 | 1.86 | 1.42 | 2.04 |
| Igsf9 | 0.38 | 0.61 | 0.62 | 1.02 |
| Igtp | −0.58 | 0.73 | 0.54 | 1.6 |
| Il13ra1 | 0.47 | 1.15 | 0.29 | −0.09 |
| Il17re | −0.83 | −0.93 | −0.32 | −2.38 |
| Il18 | 0.8 | 1.69 | 1.52 | 1.3 |
| Il18r1 | −1.2 | −1.27 | 0.39 | 0.17 |
| Il1rn | 2.35 | 2.25 | 1.36 | 0 |
| Il6st | 0.2 | 1.36 | −0.44 | −1.97 |
| Immt | 0.65 | 0.6 | −0.26 | −1.55 |
| Impa2 | 0.5 | 0.93 | 1.11 | 1.09 |
| Impact | −1.2 | −1.1 | −0.77 | −1.49 |
| Incenp | 2.79 | 2.93 | 2.48 | 2.26 |
| Inmt | −1.92 | −1.72 | −1.46 | 0 |
| Ints5 | 0.19 | 0.29 | 0.5 | 1.08 |
| Ints6 | −1.73 | −2.27 | −2.71 | −2.51 |
| Ints8 | 0.96 | 1.26 | 0.59 | −1.45 |
| Ipp | 0.33 | 0.98 | 1.07 | 0.7 |
| Iqgap3 | 1.95 | 1.97 | 2.12 | 2.19 |
| Irf1 | −0.47 | 0.07 | 0.33 | 1.03 |
| Irf2 | 0.16 | 0 | −0.03 | −1.63 |
| Irf7 | 0.29 | 0.84 | 0.36 | 1.93 |
| Irf8 | −0.81 | −0.72 | −1.03 | −1.31 |
| Irs1 | −0.18 | −0.55 | −1.08 | 0 |
| Isgf3g | 0.14 | 1.23 | 1.79 | 1.4 |
| Itgb5 | −1.12 | −0.92 | −0.96 | −1.31 |
| Itgb6 | −1.38 | −1.58 | −1.28 | −1.1 |
| Itpk1 | 0.58 | 0.35 | 0.7 | 1.08 |
| Itpr1 | −0.23 | −0.24 | −0.31 | −1.09 |
| Ivl | 0.21 | 0.01 | −0.68 | −1.85 |
| Jarid1d | −4.58 | −1 | 0 | 2.73 |
| Jmjd1c | −0.25 | 0 | −0.34 | −2.06 |
| Jtv1 | 0.5 | 0.69 | 0.55 | 1.01 |
| Jun | 2.66 | 2.98 | 2.35 | 2.35 |
| Jund1 | 0.33 | 0.17 | 0.39 | 1.6 |
| Kazald1 | −1.03 | −1.22 | −1.17 | −1.07 |
| Kbtbd11 /// | −0.69 | −1.02 | −0.95 | −0.87 |
| LOC632344 | | | | |
| Kcne1 | −0.86 | −0.88 | −2.1 | −2.28 |
| Kcnj15 | −2.67 | −3.47 | −3.34 | −1.25 |
| Kcnk5 | 0.77 | 1.28 | 1.17 | 1.62 |
| Kcnk6 | 0.71 | 1.17 | 1.88 | 1.78 |
| Kcnmb4 | −1.1 | −0.81 | −0.84 | −0.64 |
| Kcnn4 | −1.25 | −1.28 | −0.9 | −1.38 |
| Kctd12b | −2.26 | −2.7 | −1.6 | −2.25 |
| Kctd15 | −1.09 | −1.2 | −1.29 | −1.92 |
| Kctd3 | 0.88 | 1.02 | 0.89 | 0.47 |
| Kdelr3 | 0 | 3.48 | 1.62 | 1.7 |
| Khsrp | −0.2 | −0.11 | 0.38 | 1.48 |
| Kif11 | 3.5 | 5.89 | 2.77 | 2.04 |
| Kif18a | 1.54 | 1.88 | 2.29 | 2.07 |
| Kif20a | 2.92 | 2.28 | 2.8 | 2.22 |
| Kif22 | 4.37 | 2.94 | 5.47 | 2.83 |
| Kif23 | 1.59 | 1.7 | 1.98 | 1.61 |
| Kif2c | 3.49 | 2.03 | 5.62 | 2.5 |
| Kif4 | 2.63 | 2.13 | 2.1 | 2.34 |
| Kifc1 | 2.63 | 3.13 | 1.9 | 2.48 |
| Klc3 | 0.07 | 0.26 | 0.47 | 1.09 |
| Klf4 | 0.57 | 0.51 | 0.62 | 1.18 |
| Klf5 | −0.1 | 0.01 | −0.45 | −1.33 |
| Klf6 | 1.22 | 1.23 | 0.49 | 0.12 |
| Klhl14 | −4.31 | −1.57 | −1.66 | −2.69 |
| Klhl22 | −0.33 | −1.64 | −1.71 | −0.72 |
| Klhl24 | −0.51 | 0 | −0.94 | −2.12 |
| Klk6 | 0.88 | 0 | 2.14 | 1.69 |
| Klk8 | −2.11 | −2.1 | −1.4 | −1.19 |
| Kntc1 | 2.97 | 3.53 | 5.32 | 1.98 |
| Kntc2 | 2.61 | 3.33 | 3.13 | 2.72 |
| Kpna1 | 0.79 | 1.39 | 0.13 | −0.53 |
| Kpna2 /// | 1.54 | 1.48 | 1.42 | 1.37 |
| LOC670551 /// | | | | |
| LOC671639 | | | | |
| Kpna3 | 1.02 | 1.01 | 0.58 | −0.47 |
| Kpnb1 | 1.26 | 1.02 | 0.78 | −0.81 |
| Krt1-14 | 0 | 0 | 1.6 | 2.91 |
| Krt1-15 | −1.33 | −1.29 | −0.87 | −2.56 |
| Krt1-23 | 2.9 | 2.87 | 1.97 | 2.2 |
| Krt2-4 | 0.01 | 0.48 | 1.1 | 0.88 |
| Krt2-5 | −0.39 | −0.78 | −0.88 | −1.88 |
| Lactb | −1.61 | −1.76 | −1.12 | −0.89 |
| Lamb1-1 | −0.68 | −1.23 | −0.82 | −0.39 |
| Lancl3 | −0.95 | −1.55 | −1.4 | −1.83 |
| Laptm4a | −0.41 | −0.16 | −0.46 | −1.16 |
| Large | −0.96 | −1.43 | −1.14 | −0.68 |
| Lasp1 | 1.03 | 1.39 | −0.19 | −1.43 |
| Lbh | −0.72 | −1.19 | −1.16 | −0.86 |
| Lbr | 1.28 | 1.24 | 1.9 | 1.36 |
| Lcn2 | 0 | 0 | 0 | 2.37 |
| Ldha | 1.16 | 1.07 | 0.49 | 1.09 |
| Ldhb | −0.81 | −1.22 | −1.49 | −2.07 |
| Ldlrad3 | −0.51 | −0.53 | −0.84 | −1.21 |
| Ldoc1 | 1.86 | 2.81 | 2.5 | 4.09 |
| Leprel1 | −1.01 | −0.68 | −1.56 | −1.65 |
| Lgals1 | −0.99 | −1.33 | −1.33 | −0.16 |
| Lgals3 | 0.41 | 0 | 0.87 | −1.08 |
| Lig1 | 1.9 | 1.97 | 1.96 | 1.65 |
| Lima1 | 0.32 | 0.44 | −0.58 | −1.65 |
| Limd1 | −0.04 | −0.39 | 0.35 | 1.01 |
| Limk2 | 0.05 | 0.05 | −0.39 | −1.15 |
| Lims1 | 0.52 | 1.01 | −0.1 | −2.03 |
| Lin7c | 0.76 | 0.83 | −0.11 | −1.86 |
| Lin9 | 1.12 | 1.62 | 2.46 | 1.67 |
| Litaf | 0.84 | 1.03 | 0.23 | 0.35 |
| Lman2 | −0.54 | −0.2 | −0.48 | −1.06 |
| Lmbr1 | −0.29 | −0.07 | −0.71 | −1.65 |
| Lmnb1 | 2.19 | 1.64 | 1.79 | 1.45 |
| Lmo1 | −0.88 | −1.19 | −1.48 | −1.75 |
| Lonrf2 | −0.5 | −0.55 | −1.28 | −1.36 |
| Lonrf3 | 1.25 | 2.28 | 0.99 | 1.07 |
| Lpin1 | −1.01 | −0.94 | −1.32 | −2.5 |
| Lpl | −0.57 | 0.24 | 0.21 | 1.32 |
| Lpp | −0.61 | −1.42 | −0.73 | −0.28 |
| Lrp1 | −0.45 | −0.89 | −0.96 | −1.09 |
| Lrrc33 | −2.71 | −1.74 | −1.28 | −4.25 |
| Lrrc50 | 0 | 0 | 1.52 | 1.15 |
| Lrrc51 | −1.81 | −1.25 | −1.55 | −1.99 |
| Lrrc54 | −0.82 | −1.52 | −1.04 | −1.32 |
| Lrrc8e | −0.38 | −0.27 | −0.43 | −1.14 |
| Lsm2 | 1.27 | 1.61 | 1.27 | 1.25 |
| Lsm3 | 0.75 | 0.8 | 0.86 | 1.14 |
| Lsm5 | 1.04 | 0.89 | 0.82 | 1.13 |
| Lsm8 | 1.01 | 0.87 | 0.82 | 1.51 |
| Luc7l2 | −0.63 | −1.9 | −1.14 | −2.49 |
| Lum | −1.9 | −1.78 | −1.9 | 0 |
| Luzp1 | −0.44 | −0.44 | 0.31 | 1.06 |
| Luzp5 | 2.39 | 2.52 | 2.92 | 2.44 |
| Ly6c | −0.76 | −0.18 | −0.16 | 1.25 |
| Ly6e | −0.81 | 0.23 | 0.8 | 1.02 |
| Lynx1 | −1 | −0.85 | −0.3 | −0.36 |
| Lypla1 | 0.35 | 0.5 | −0.46 | −1.56 |
| Lyzs | −0.36 | 0.32 | 0.23 | 1.41 |
| Lzic | 1.39 | 0.87 | 0.82 | 0.55 |
| Lzp-s | −0.61 | 0.25 | 0.29 | 2.13 |
| Mad2l1 | 3 | 2.96 | 3.11 | 2.34 |
| Mafb | −1.08 | −0.89 | −1.27 | −1.19 |
| Maff | 1 | 1.64 | 0.94 | 1.52 |
| Magoh | 0.64 | 0.81 | 0.84 | 1.08 |
| Malat1 | −1.14 | −1.56 | −0.31 | 0.27 |
| Man1a | −0.48 | −0.35 | −0.54 | −1.03 |
| Man1a2 | 0.2 | 0 | 0 | −2.51 |
| Man1c1 | −1.37 | −1.26 | −0.84 | −0.24 |
| Map4k4 | 0.42 | 0.22 | 0.96 | 1.05 |
| Map4k5 | 1.07 | 1.23 | 0.29 | −0.64 |
| Mapk14 | 0.3 | 1.11 | 0.23 | −1.69 |
| Mapk1ip1 | −0.76 | −1 | −0.62 | −0.83 |
| Mapk6 | 0.91 | 1.28 | 0.46 | 0 |
| Mapkapk5 | 0.28 | 0.49 | 0.76 | 1.12 |
| Mapre2 | −1.3 | −1.53 | −1.42 | −0.25 |
| Marcksl1 | −0.25 | 1.61 | −0.79 | −0.13 |
| Marveld1 | −1.33 | −0.92 | −0.96 | 0.06 |
| Mast2 | 0.73 | 0.74 | 1.22 | 1.26 |
| Mast4 | −0.5 | 0 | −0.82 | −2.77 |
| Mastl | 2.63 | 2.35 | 2.49 | 1.32 |
| Maz | 0.29 | 0.41 | 1.19 | 1.61 |
| Mbd4 | 1.21 | 1.23 | 1.38 | 1.01 |
| Mbd5 | −0.73 | −0.67 | −0.47 | −1.16 |
| Mbnl2 | 0.17 | 0.1 | −0.21 | −1.55 |
| Mbtd1 | −0.09 | 0 | −0.79 | −1.44 |
| Mcm10 | 2.7 | 2.33 | 2.13 | 2.09 |
| Mcm2 | 2.05 | 2.29 | 2.09 | 2.24 |
| Mcm3 /// | 2.9 | 2.55 | 2.86 | 2.71 |
| LOC671904 | | | | |
| Mcm4 | 1.86 | 2.17 | 1.54 | 2.1 |
| Mcm5 | 3.25 | 3.28 | 2.81 | 2.56 |
| Mcm6 | 3.7 | 4.33 | 3.34 | 3.46 |
| Mcm7 | 1.39 | 1.41 | 1.33 | 1.55 |
| Mcm8 | 1.26 | 2.06 | 1.79 | 0.8 |
| Mcpt1 | 0 | 0 | 0 | 4.69 |
| Mcpt2 | 0 | 0 | 0 | 5.13 |
| Mdm4 | −0.15 | −0.12 | −0.36 | −1.1 |
| Me2 | 0.78 | 0.89 | 1.13 | 1.03 |
| Mef2a | −0.36 | −0.2 | −0.23 | −1.04 |
| Meis1 | −0.5 | 0 | −0.6 | −3 |
| Melk | 3.7 | 3.17 | 4.49 | 2.61 |
| Mertk | −0.28 | −0.53 | −1.21 | −0.82 |
| Met | 0.84 | 1.29 | 0.22 | −1.57 |
| Metrn | 1.38 | 0.69 | 1.14 | 1.19 |
| Mettl7a | −0.67 | −1.12 | −0.89 | −1.67 |
| Mettl7a /// Ubie | −1.34 | −0.86 | −1.34 | −1.16 |
| Mettl7a /// Ubie | −0.39 | −0.27 | −0.94 | −1.28 |
| /// LOC554292 | | | | |
| Mfap3l | 0.05 | −0.25 | −0.76 | −1.75 |
| Mfhas1 | −1.1 | −1.63 | −0.78 | −0.3 |
| MGC73635 | 2.96 | 2.41 | 2.95 | 1.94 |
| Mgp | −2.27 | −1.75 | −1.21 | −0.57 |
| Mia3 | −0.86 | −1.49 | −0.24 | 0.41 |
| Mier1 | 0.86 | 1.09 | 0.82 | −0.9 |
| Mki67 /// | 4.04 | 2.31 | 4.24 | 2.32 |
| LOC638774 | | | | |
| Mlf1 | 2.91 | 2.03 | 3.32 | 2.62 |
| Mlf1ip | 3.46 | 1.16 | 1.59 | 1.3 |
| Mlkl | 1.93 | 1.33 | 1.5 | 1.21 |
| Mllt4 | 0.01 | 0.29 | 1.18 | 1.05 |
| Mlstd2 | 1.77 | 2.03 | 0.23 | −1.69 |
| Mlycd | −0.61 | −1.07 | −0.7 | −0.74 |
| Mlze | 1 | 1.06 | 1.2 | 1.33 |
| Mmp15 | −1.05 | −0.75 | 0.28 | 0.04 |
| Mmp2 | −3.54 | −1.33 | −0.85 | 0 |
| Mmp7 | 0 | 0 | 0 | 2.51 |
| Mnt | −0.53 | −1.04 | −0.12 | 0.07 |
| Mobk1b | 0.88 | 1.68 | −0.06 | −1.94 |
| Mocs1 | −0.8 | −1.29 | −1.53 | −1.48 |
| Mon1b | −0.9 | −1.17 | −1.07 | −0.84 |
| Mphosph1 | 2.46 | 2.77 | 2.51 | 1.98 |
| Mpzl1 | 0.13 | 0.69 | 0.77 | 1.41 |
| Mr1 | −0.5 | −0.56 | −1.15 | −2.51 |
| Mre11a | 1.08 | 1.24 | 1.01 | 0.81 |
| Mrpl41 | 0.31 | 0.11 | 0.34 | 1.02 |
| Mrpl9 | 0.38 | 0.47 | −0.21 | −1.37 |
| Mrps2 | 0.48 | 1.03 | −0.22 | −1.01 |
| Mrps23 | 0.38 | 0.49 | −0.47 | −1.15 |
| Msh6 | 1.01 | 0.9 | 1.03 | 1.25 |
| Msi2 | −0.31 | 0 | −0.36 | −2.8 |
| Msrb2 | −1.07 | −1.93 | −1.54 | −4.56 |
| Msrb3 | 0.74 | 1.83 | 1.44 | 1.33 |
| Mtac2d1 | −1.9 | −0.61 | −0.28 | −1.7 |
| Mtap4 | 0.11 | 0.27 | 0.62 | 1.02 |
| Mthfd1l | 1.2 | 1.42 | 0.93 | 1.48 |
| Mthfd2 | 2.14 | 1.35 | 2.36 | 2.43 |
| Mtm1 | 2.19 | 1.81 | 1.57 | −0.01 |
| Mtmr2 | 0.45 | 1.01 | 0.17 | −0.41 |
| Mtmr4 | −0.5 | −0.03 | −0.43 | −1.13 |
| Mtmr6 | 0.17 | 0.6 | −0.07 | −1.13 |
| Mtss1 | 0.19 | 0.06 | −0.47 | −1.07 |
| Mtus1 | −0.26 | 0.4 | −0.28 | −1.22 |
| Mxd1 | 0.44 | 0.87 | 0.8 | 1.36 |
| Mxd3 | 0 | 0 | 1.01 | 2.39 |
| Mxd4 | −1.2 | −0.92 | −0.69 | −0.4 |
| Mxi1 | 0.66 | 1.33 | −0.19 | −1.43 |
| Mxra8 | −1.29 | −0.62 | −0.72 | 0.03 |
| Mybl2 | 1.8 | 2.11 | 1.74 | 1.92 |
| Mycl1 | 0.45 | 0.85 | −0.05 | −1.63 |
| Myef2 | 0.69 | 0.63 | 1.04 | 1.28 |
| Myh11 | −4.14 | −0.51 | −1.82 | 0 |
| Myl6 | −0.82 | −0.56 | −0.49 | −1.52 |
| Myl9 | −2.46 | −1.24 | −2 | 0 |
| Mylip | −0.89 | −1.04 | −0.85 | −1.15 |
| Mylk | −1.6 | −1.35 | −0.67 | −0.98 |
| Nab2 | 0.97 | 1.33 | 1.59 | 1.88 |
| Nalp10 | 2.35 | 2.27 | 1.07 | 1.35 |
| Nans | 0.49 | 0.78 | 1.06 | 1.83 |
| Nap1l1 | 1.01 | 1.15 | 0.96 | 0.42 |
| Napa | −0.65 | −1.04 | −0.88 | −1.22 |
| Nasp | 1.96 | 1.81 | 2.22 | 1.94 |
| Nat11 | 0.75 | 1.05 | 0.66 | 0.19 |
| Nav1 | −1.4 | −1.03 | −0.96 | −0.92 |
| Nbeal1 | 0.06 | 0 | 0.07 | −2.02 |
| Nbr1 | 0.29 | 0.64 | −0.41 | −1.59 |
| Ncf1 | −1.19 | −1.69 | −1.74 | −2.21 |
| Ncoa4 /// | 1.23 | 1.33 | −0.08 | −1.21 |
| LOC627557 | | | | |
| Nde1 | 0.85 | 1.33 | 0.74 | 0.55 |
| Ndn | −2.12 | −1.84 | −2.09 | −4.53 |
| Ndrg1 | 0.88 | 0.63 | −0.11 | 1.26 |
| Ndrg1 /// Ndrl | 1.44 | 0.69 | −0.35 | 0.39 |
| Ndrg3 | −0.39 | −0.24 | −0.59 | −1.26 |
| Ndrl | 0.99 | 0.78 | −0.04 | 1.1 |
| Ndufab1 | −1.04 | 0.05 | −0.44 | 0.21 |
| Nedd1 | 0.87 | 1.64 | 0.68 | 0 |
| Nedd4 | 0.1 | 0.27 | −0.45 | −1.02 |
| Nedd4l | −0.62 | 0 | −0.29 | −2.51 |
| Nek2 | 3.62 | 3.28 | 4.31 | 3.02 |
| Nek3 | −1.06 | −0.93 | −0.98 | −1.02 |
| Nelf | −0.27 | 0.02 | −1.06 | 0.45 |
| Net1 | 0.66 | 1.1 | 0.19 | −0.67 |
| Nfat5 | −0.2 | −0.98 | −0.92 | −2.56 |
| Nfia | −0.89 | −1.34 | −0.28 | −0.69 |
| Nfib | 0.07 | 0.01 | 0.84 | 1.32 |
| Nfix | −1.26 | −1.23 | −0.82 | 0.16 |
| Nfkbia | −0.13 | −0.08 | 0.48 | 1.19 |
| Nfkbil2 | 0.99 | 0.87 | 1.13 | 1.48 |
| Nfs1 | 0.68 | 1 | 0.08 | −0.79 |
| Ngfrap1 | 0.73 | 0.83 | 0.84 | 1.25 |
| Nipa1 | −0.87 | −0.71 | −0.68 | −1.05 |
| Nipsnap1 | −0.48 | −1.19 | −1.1 | −0.76 |
| Nisch | −0.52 | −0.97 | −0.28 | −1.43 |
| Nlk | −0.13 | 0 | −0.85 | −3.12 |
| Nme4 | −1.32 | −1.48 | −0.4 | −0.53 |
| Nnt | −0.86 | −0.89 | −1.45 | −0.62 |
| No1l1 | 0.92 | 0.57 | −0.19 | −1 |
| Nol5 | 1.11 | 0.75 | 1.02 | 0.99 |
| Nol5a | 0.83 | 0.5 | 0.79 | 1.31 |
| Nono | −0.39 | 0 | 0 | −1.8 |
| Nope | −1.14 | −0.5 | −1.06 | −1.64 |
| Notch1 | −0.94 | −1.33 | −0.6 | −0.32 |
| Nptn | 0.11 | 0 | 0.28 | −1.48 |
| Nqo1 | −0.63 | −1.4 | −1.47 | −2.21 |
| Nr2c1 | −0.39 | 0.22 | −0.61 | −1.58 |
| Nrarp | −0.43 | −0.55 | −0.6 | −1.19 |
| Nrip1 | −0.23 | 0 | 0.19 | −2.45 |
| Nrm | 2.3 | 2.29 | 2.23 | 1.61 |
| Nrtn | −1.35 | −0.68 | −0.45 | −0.17 |
| Nsg1 | 1.47 | 1.24 | 1.5 | 2.44 |
| Nsun4 | −0.75 | −0.67 | −0.05 | −1.02 |
| Nubp1 | 0.98 | 1.9 | 0.1 | −0.71 |
| Nucb2 | 1.2 | 0.75 | 0.68 | 0.87 |
| Nucks1 | 0.28 | 0.21 | 0.84 | 1.17 |
| Nudcd1 | 1.03 | 1.46 | 0.55 | −0.59 |
| Nudt16 | −0.86 | −1.26 | −0.81 | −0.35 |
| Nup107 | 1.04 | 1.07 | 1.56 | 1.47 |
| Nup133 | 0.73 | 1.24 | 0.48 | −0.16 |
| Nup155 | 1.32 | 1.02 | 1.34 | 0.94 |
| Nup210 | 0.36 | 1.21 | 1.13 | 1.53 |
| Nup35 | 0.86 | 1.07 | 0.78 | 0.78 |
| Nup37 | 0.78 | 1.03 | 0.86 | 1.01 |
| Nup43 | 1.11 | 0.96 | 0.4 | 0.63 |
| Nup54 | 0.7 | 1 | 0.9 | 1.28 |
| Nup62 | 0.77 | 0.66 | 1.21 | 1.68 |
| Nup85 | 0.97 | 0.91 | 0.42 | 1.35 |
| Nupl1 | 0.98 | 1.66 | 0.69 | 0.16 |
| Nupr1 | 0.62 | 1.3 | 1.28 | 2.34 |
| Nusap1 | 3.4 | 3.12 | 3.7 | 2.19 |
| Nxt1 | 1.11 | 0.84 | 0.94 | 1.07 |
| Oas1a | 0.93 | 1.89 | 3.12 | 3.04 |
| Oasl2 | 0 | 1.16 | 2.13 | 4.51 |
| Odc1 /// | 0.83 | 1.04 | 0.75 | 1.1 |
| LOC639787 /// | | | | |
| LOC666231 /// | | | | |
| LOC668343 /// | | | | |
| LOC670279 /// | | | | |
| LOC671154 /// | | | | |
| LOC671973 /// | | | | |
| LOC673302 | | | | |
| Odc1 /// | 0.27 | 0.43 | 0.77 | 1.1 |
| LOC666231 /// | | | | |
| LOC668343 /// | | | | |
| LOC670279 /// | | | | |
| LOC671973 /// | | | | |
| LOC673302 | | | | |
| Odz4 | 2.77 | 2.46 | 0 | 1.61 |
| Ogt | −0.19 | −0.12 | −0.42 | −2.2 |
| Oip5 | 2.78 | 2.53 | 1.97 | 2.24 |
| Oit1 | −0.21 | −0.35 | −0.39 | −1.37 |
| Olfm1 | −1.6 | −1.41 | −1.75 | −1.91 |
| Opn3 | 0.39 | 0.43 | 1.42 | 1.94 |
| Orc1l | 4.09 | 1.46 | 3.11 | 2.65 |
| Orc6l | 1.54 | 1.37 | 1.51 | 1.5 |
| Ormdl3 | −0.73 | −0.69 | −0.79 | −1.03 |
| Osbpl3 | −0.93 | −1.18 | −0.78 | −1.5 |
| Osbpl8 | −0.85 | −0.51 | −1.09 | −0.82 |
| Osmr | −0.46 | 0.03 | −1.08 | −2.24 |
| Ostm1 | −0.49 | −0.63 | −0.6 | −1.03 |
| Otub2 | −0.57 | −0.67 | −0.7 | −1.21 |
| Otud4 | 0.46 | 1.25 | 0.33 | −0.59 |
| P2rx4 | −0.96 | −1.08 | −1.12 | −0.44 |
| Pabpn1 | −0.67 | −0.91 | −0.52 | −1.59 |
| Pacs2 | 0.22 | 0.69 | 1.19 | 0.78 |
| Pacsin1 | −0.91 | −1.33 | −0.52 | −1.03 |
| Padi3 | 0.54 | 0.71 | 1.14 | 0.1 |
| Pafah1b2 | 0.69 | 0.55 | −0.29 | −1.67 |
| Pak3 | −0.42 | −1.38 | 0.9 | −0.5 |
| Pam | −0.66 | −0.69 | −1.35 | −0.61 |
| Papola | −0.55 | −0.58 | −0.45 | −1.56 |
| Paqr3 | −1 | −0.82 | −0.72 | −0.84 |
| Paqr4 | 0 | 1.12 | 2.72 | 3.02 |
| Paqr5 | −0.2 | −0.16 | −0.44 | −1.33 |
| Paqr6 | −1.72 | −1.04 | −1.02 | −1.31 |
| Paqr7 | −0.98 | −1.09 | −1.04 | −1.1 |
| Paqr8 | −0.11 | 0.7 | 1.25 | 0.7 |
| Pard3 | −0.2 | −0.42 | −0.83 | −1.52 |
| Pard6b | 0.68 | 1.12 | 0.79 | 1.34 |
| Parp3 | −1.82 | −1.08 | −0.68 | −0.76 |
| Parp4 | −0.86 | −1.02 | −0.42 | −0.24 |
| Pask | 1.55 | 1.14 | 3.98 | 1.91 |
| Paxip1 | 0.85 | 0.09 | 1.33 | 0.95 |
| Pbk | 3.45 | 3.62 | 4.48 | 2.76 |
| Pbx1 | −0.03 | −0.57 | −1.06 | −2.25 |
| Pbx3 | 0.81 | 0.88 | 1.33 | 1.78 |
| Pcaf | −0.54 | 0 | 0.02 | −1.31 |
| Pcdhgc3 | −0.16 | 0 | −0.38 | −2.09 |
| Pcgf3 | 0.06 | 0 | 0.12 | −2.29 |
| Pcgf6 | −0.68 | −1.36 | −0.91 | −2.14 |
| Pcmt1 | 1.31 | 0.69 | 1.31 | 1.68 |
| Pcmtd1 | −0.42 | −0.15 | −0.36 | −1.01 |
| Pcmtd2 | −0.21 | −0.18 | −0.38 | −1.04 |
| Pcna | 1.37 | 1.43 | 1.43 | 1.52 |
| Pcnt | 0.59 | 2.21 | 0.85 | −0.36 |
| Pcolce | −1.48 | −1.26 | −0.7 | −0.94 |
| Pctp | −0.86 | −0.68 | −0.81 | −1 |
| Pcyox1 | −0.65 | −0.4 | −0.58 | −1.11 |
| Pdap1 | 0.31 | −0.35 | 0.47 | 1.23 |
| Pdcd6ip | 0.87 | 1.79 | −0.05 | −0.88 |
| Pde2a | −0.7 | −1.09 | −0.38 | 0.09 |
| Pde7a | 1.06 | 1.1 | 0.89 | −0.65 |
| Pdgfra | −2.13 | −2.12 | 0 | 0 |
| Pdha1 | −0.14 | −0.03 | −0.65 | −1.43 |
| Pdik1l | 0.77 | 2.93 | 0.83 | −0.23 |
| Pdk4 | −0.29 | −0.31 | −1.03 | −1.13 |
| Pdlim1 | 0.59 | 0.56 | 0.9 | 1.04 |
| Pdlim5 | 0.96 | 0 | 0.64 | −1.76 |
| Pdxk | −1.05 | −1.13 | −0.63 | −0.5 |
| Pdzd2 | −0.63 | −0.51 | −0.63 | −1.16 |
| Pdzrn3 | −2.11 | −1.05 | −0.47 | −0.55 |
| Peg3 | 0.31 | 0.3 | −0.15 | −1.84 |
| Peli1 | 0.57 | 0.75 | −0.89 | −2.5 |
| Pex12 | −0.56 | −0.28 | −0.88 | −1.06 |
| Pex3 | −0.19 | −0.3 | −0.43 | −1.27 |
| Pfdn4 | 0.67 | 0.31 | 0.68 | 1.19 |
| Pfdn5 | −0.38 | −0.41 | −0.79 | −1.17 |
| Pfkfb3 | 2.17 | 1.5 | 0 | 0 |
| Pfkfb4 | −1.46 | −1.35 | −0.49 | −1.27 |
| Pfkm | −0.9 | −0.76 | −0.85 | −1.03 |
| Pfkp | 1.85 | 1.55 | 0.94 | 2.16 |
| Pftk1 | −1.06 | −0.53 | 0.29 | 0.31 |
| Pgcp | −2.61 | −2.46 | −1.97 | −2.71 |
| Pgk1 /// | 2.45 | 1.56 | 0.34 | 2.48 |
| LOC433594 /// | | | | |
| LOC665053 /// | | | | |
| LOC668435 | | | | |
| Pgk1 /// | 1.32 | 1.1 | 0.5 | 1.27 |
| LOC433594 /// | | | | |
| LOC668435 | | | | |
| Phactr4 | 0.57 | 0.56 | 0.56 | 1.09 |
| Phf10 | 0.52 | 1.02 | 0.75 | 0.98 |
| Phf13 | 0.11 | 0.04 | 0.41 | 1.01 |
| Phf14 | −0.09 | 0 | −0.32 | −1.77 |
| Phf15 | −0.94 | −0.92 | −1.04 | −0.84 |
| Phf17 | 0.47 | 1.13 | 0.44 | 0.01 |
| Phf20l1 | 0.3 | −0.53 | −0.36 | −1.64 |
| Phf6 | 0.83 | 1.16 | 0.34 | −0.34 |
| Phgdh /// | 1.02 | 0.47 | 0.67 | 0.59 |
| LOC666422 /// | | | | |
| LOC666875 /// | | | | |
| LOC669985 /// | | | | |
| LOC671102 /// | | | | |
| LOC673015 /// | | | | |
| LOC675010 | | | | |
| Phgdh /// | 0.56 | 0.07 | 0.59 | 1.41 |
| LOC668771 /// | | | | |
| LOC670155 /// | | | | |
| LOC671972 /// | | | | |
| LOC673015 | | | | |
| Phgdh /// | 0.41 | −0.03 | 0.51 | 1.58 |
| LOC668771 /// | | | | |
| LOC671972 /// | | | | |
| LOC673015 | | | | |
| Phkb | −0.93 | −1.05 | −1.2 | −0.98 |
| Phtf2 | −1.04 | −0.71 | −0.93 | −0.97 |
| Pi16 | −1.75 | −3.63 | −1.65 | 0 |
| Pi4k2b | 1.03 | 0.8 | 0.31 | −0.12 |
| Piga | 1.49 | 0.8 | 1.33 | 0.35 |
| Pigr | −1.33 | −0.27 | 1.35 | 1.68 |
| Pik3ap1 | −1.08 | 0 | 0 | 1.51 |
| Pik3r1 | −0.5 | −0.3 | −0.85 | −2.84 |
| Pik4ca | −0.8 | −1.03 | −0.6 | −0.79 |
| Pim1 | 0.41 | 0.87 | 0.56 | 1.47 |
| Pim3 | −0.14 | 0.18 | 0.45 | 1.11 |
| Pip5k2c | 0.2 | 0.4 | −0.07 | −1.25 |
| Pitpnb | 0.93 | 1.16 | −0.26 | −1.3 |
| Pkd2 | −0.84 | −0.7 | −1.06 | −0.31 |
| Pkhd1 | −1.19 | −0.99 | −0.57 | −0.46 |
| Pkmyt1 | 2.38 | 1.58 | 1.57 | 1.8 |
| Pkp1 | 0.42 | 0.84 | 1.22 | 1.09 |
| Pkp2 | 0.59 | 0.5 | 0.58 | 1.04 |
| Pkp3 | 0.53 | 1.19 | 0.04 | −0.57 |
| Pla2g6 | −0.86 | −0.48 | −0.88 | −1.14 |
| Pla2g7 | −1.07 | −1.07 | −1.37 | −2.89 |
| Pla2r1 | −1.45 | −1.08 | −0.21 | −0.8 |
| Plac1 | 0 | 0 | 0 | 2.42 |
| Plac8 | −0.66 | −0.68 | −1 | 1.13 |
| Plau | −1.5 | −1.99 | −1.49 | −1.7 |
| Plaur | 1.76 | 1.73 | 1.15 | 1.12 |
| Plce1 | −1.01 | −0.69 | −0.62 | −0.71 |
| Plch1 | 2.97 | 1.2 | 2.2 | 0.79 |
| Pld1 | −0.48 | −0.83 | −0.75 | −1.31 |
| Pldn | 0.39 | 0.83 | −0.45 | −1.47 |
| Plekha2 | 0.83 | 1.05 | −0.05 | −0.35 |
| Plekha8 | 0.84 | 0.47 | 0.95 | 1.34 |
| Plekhb1 | −2.08 | −2.59 | −2.86 | −2.26 |
| Plekhc1 | −1.28 | −1.24 | −0.74 | −0.83 |
| Plekhf2 | 0.66 | 1.53 | 0.45 | −0.16 |
| Plekhg2 | 0.4 | 0.36 | 0.48 | 1.16 |
| Plk1 | 3 | 3.84 | 2.85 | 2 |
| Plk2 | 0.47 | 0.57 | 1.01 | 1.65 |
| Plk4 | 3.09 | 4.9 | 2.87 | 2.49 |
| Pls3 | −0.67 | −0.68 | −1.12 | −0.96 |
| Plscr1 | 1.48 | 1.36 | 0.42 | 1.91 |
| Plscr1 /// | 1.16 | 0 | 0.75 | 1.59 |
| LOC433328 /// | | | | |
| LOC677340 | | | | |
| Pltp | −0.65 | −1.05 | −1.21 | −2.38 |
| Plvap | −0.17 | 0.13 | 0.79 | 1.92 |
| Pmaip1 | 1.66 | 1.87 | 1.35 | 1.92 |
| Pmf1 | 1.88 | 0.53 | 0.53 | 1.52 |
| Pmm2 | 0.4 | 1.06 | −0.11 | −1.72 |
| Pmp22 | −1.59 | −0.78 | −1.14 | 0 |
| Pms1 | 0.82 | 1.05 | 0.9 | 0.8 |
| Pms2 | 0.96 | 0.96 | 1.14 | 0.85 |
| Pnn | 0.73 | 1.44 | 0.18 | −1.71 |
| Pola1 | 2.24 | 2.12 | 2.21 | 1.41 |
| Pola2 | 1.66 | 1.72 | 1.24 | 1.42 |
| Polb | −0.08 | 0 | −0.32 | −3.11 |
| Pold1 | 1.75 | 1.7 | 1.45 | 2.32 |
| Pold2 | 0.96 | 1.12 | 0.81 | 1.32 |
| Pold3 | 1.18 | 0.84 | 0.47 | 0.16 |
| Pole | 3.75 | 3.12 | 5.21 | 1.84 |
| Pole2 | 1.88 | 1.71 | 2.18 | 1.68 |
| Pole3 | 1.08 | 1.25 | 0.83 | 0.5 |
| Polg | 0.11 | 0.69 | 0.85 | 1.24 |
| Polr3c | 0.61 | 0.83 | −0.25 | −1.11 |
| Pon3 | 1.18 | 1.3 | 0.87 | 0.89 |
| Postn | −1.44 | 0 | −2.09 | 0 |
| Ppap2b | −0.94 | −1.3 | −0.72 | −1.29 |
| Ppara | −0.52 | −0.99 | −1.09 | −1.17 |
| Ppfibp2 | −0.27 | −0.26 | −0.48 | −1.03 |
| Ppih /// | 1.05 | 0.38 | 1 | 0.71 |
| LOC433064 /// | | | | |
| LOC624822 /// | | | | |
| LOC629952 /// | | | | |
| LOC665989 /// | | | | |
| LOC666411 /// | | | | |
| LOC667598 /// | | | | |
| LOC669441 /// | | | | |
| LOC670964 /// | | | | |
| LOC671009 /// | | | | |
| LOC676493 | | | | |
| Ppih /// | 1.07 | 0.98 | 1.34 | 1.21 |
| LOC433064 /// | | | | |
| LOC624822 /// | | | | |
| LOC665989 /// | | | | |
| LOC667598 /// | | | | |
| LOC669441 /// | | | | |
| LOC670964 /// | | | | |
| LOC676493 | | | | |
| Ppih /// | 1.12 | 1.05 | 1.41 | 1.16 |
| LOC433064 /// | | | | |
| LOC629952 /// | | | | |
| LOC665989 /// | | | | |
| LOC666411 /// | | | | |
| LOC669441 /// | | | | |
| LOC671009 /// | | | | |
| LOC676493 | | | | |
| Ppil1 | 0.86 | 1.03 | 0.83 | 0.59 |
| Ppil5 | 5.43 | 5.81 | 4.42 | 3.32 |
| Ppm1b | 0.69 | 1.3 | 0.36 | 0.2 |
| Ppm1h | −0.6 | −0.82 | −1.02 | −0.94 |
| Ppm11 | −1.07 | −0.25 | −0.02 | −0.34 |
| Ppm1m | −0.19 | 0.17 | 0.26 | 1.27 |
| Ppp1r12a | 0.46 | 1.01 | −0.12 | −1.58 |
| Ppp1r12b | −1.35 | −0.99 | −0.78 | −1.87 |
| Ppp1r2 | 0.83 | 1.08 | 0.41 | 0.03 |
| Ppp1r8 | 0.52 | 0.71 | 0.59 | 1.3 |
| Ppp2r5e | −0.06 | 0 | 0.13 | −2.43 |
| Prc1 | 3.97 | 3.03 | 3.78 | 1.98 |
| Prdm4 | 0.19 | 0.37 | 0.14 | 1.02 |
| Prdx4 | 1.21 | 1.62 | 1.22 | 1.9 |
| Prim1 | 3.12 | 2.9 | 2.6 | 2.06 |
| Prim2 | 1.96 | 2.55 | 1.84 | 1.68 |
| Prkar2a | 0.28 | 0.05 | 0.75 | 1.07 |
| Prkar2b | 1.2 | 1.39 | 0.48 | −0.13 |
| Prkch | 0.02 | 0.26 | 0.19 | −1.04 |
| Prkci | 1.25 | 2.18 | 0.44 | −0.78 |
| Procr | −0.81 | −0.97 | −1.03 | −0.52 |
| Prodh /// | −1.02 | −1.4 | −1.01 | −1.53 |
| LOC671160 | | | | |
| Prom2 | −0.09 | −0.05 | −1.13 | −2.19 |
| Prpf3 | 0.49 | 0.26 | 0.84 | 1.47 |
| Prpf4b | 0.48 | 0.45 | 0.05 | −1.01 |
| Prps1 /// | 0.84 | 1.02 | 0.25 | −0.46 |
| AU021838 | | | | |
| Prss15 | 1.04 | 0.99 | 0.5 | 0.87 |
| Prss23 | −0.95 | 0 | −1.42 | 0.58 |
| Prss27 | 0.36 | 0.15 | 1.26 | 1.17 |
| Psap | −0.4 | −0.1 | −0.46 | −1.42 |
| Psat1 | 1.77 | 1.44 | 1.22 | 0.82 |
| Psca | −1.12 | −0.88 | −0.86 | −1.32 |
| Psen1 | 0.07 | 0.35 | −0.08 | −1.15 |
| Psip1 | 0.71 | 1.04 | 1.21 | 0.99 |
| Psmb9 | 0.04 | 0.6 | 0.87 | 1.43 |
| Psme4 | 0.51 | 0.54 | −0.53 | −1.23 |
| Psrc1 | 1.33 | 3.21 | 1.91 | 1.33 |
| Pstpip2 | 1.16 | 0.78 | 1.1 | 0.5 |
| Ptbp2 | 0.19 | 0 | −0.19 | −3.49 |
| Ptdsr | 1.32 | 1.33 | 0.05 | 0.75 |
| Ptk9 | 0.38 | 0.78 | −0.39 | −1.43 |
| Ptk9l | −0.06 | 0.14 | 0.66 | 1.03 |
| Ptov1 | −0.51 | −0.55 | 0.74 | 1.14 |
| Ptp4a1 | −0.08 | 0.64 | −0.24 | −1.04 |
| Ptp4a2 | −0.58 | −0.83 | 0.34 | 1.06 |
| Ptplad1 | −0.24 | 0.84 | −0.22 | −1.57 |
| Ptplb | −1.15 | −1.32 | −1.4 | −2.01 |
| Ptprb | −0.58 | −1.29 | 0 | 0 |
| Ptpre | −1.26 | −1.39 | −0.96 | −1.73 |
| Ptprf | 0.09 | 0.15 | 0 | −1.21 |
| Ptprj | 0.51 | 0.62 | 0.95 | 1.32 |
| Ptprr | 0.51 | 1.03 | 0.38 | −0.26 |
| Ptpru | −1 | −1.45 | −1.3 | −1.2 |
| Pttg1 | 1.72 | 1.48 | 1.52 | 1.19 |
| Pvrl2 | 0.79 | 0.59 | 0.67 | 1.05 |
| Pvrl3 | −1.08 | −1.5 | −0.53 | −2.08 |
| Rab10 | −0.41 | −0.68 | −0.66 | −1.25 |
| Rab14 | 0.24 | 0 | 0.15 | −2.66 |
| Rab17 | 0 | 0 | 0.43 | 1.8 |
| Rab18 | 0.28 | 0.7 | −0.64 | −2.13 |
| Rab27a | −0.86 | −0.53 | −1.1 | −1.35 |
| Rab27b | −0.34 | −0.11 | −0.76 | −2.03 |
| Rab3b | 1.8 | 0.64 | 1.13 | 1.06 |
| Rab6ip2 | 0.71 | 0.76 | 0.67 | 1.1 |
| Racgap1 | 5.85 | 2.66 | 5.49 | 2.77 |
| Rad18 | 1.84 | 1.19 | 1.56 | 1.04 |
| Rad21 | 1.07 | 1.54 | 0.85 | −0.24 |
| Rad23a | 0.61 | 1.09 | −0.22 | −0.45 |
| Rad51 | 3.62 | 5.06 | 4.32 | 2.72 |
| Rad51ap1 | 2.9 | 5.41 | 4.8 | 2.85 |
| Rad54l | 2.1 | 2.72 | 2.29 | 2.53 |
| Rai14 | −0.25 | 0 | −0.88 | −4.65 |
| Ranbp1 | 1.13 | 0.89 | 0.94 | 0.84 |
| Rangap1 | 0.94 | 1.19 | 0.53 | −0.18 |
| Rap2c | 0.29 | 0.29 | 0.64 | 1.33 |
| Rarg | 0.15 | 1.01 | 0.32 | −0.71 |
| Rarres2 | −1.38 | −0.92 | −2.06 | 0 |
| Rasa3 | −0.66 | −1.17 | −0.82 | −0.74 |
| Rasgrp1 | 0 | 0 | 0 | 1.65 |
| Rasl11b | −1.43 | −1.43 | −1.32 | −1.73 |
| Rassf1 | 0.31 | 0.48 | 0.45 | 1.06 |
| Rassf3 | −1.15 | −0.74 | −0.28 | −0.29 |
| Rbbp4 | 0.54 | 1.35 | 0.43 | −0.99 |
| Rbl1 | 1.4 | 1.46 | 1.31 | 1.57 |
| Rbl2 /// | −0.45 | −0.1 | −0.87 | −1.39 |
| LOC635075 | | | | |
| Rbm14 | 0.61 | 0.06 | 0.36 | 1.12 |
| Rbm5 | −0.46 | −1.53 | −0.31 | −1.36 |
| Rbm6 | 0.25 | 0.27 | 0.04 | −1.2 |
| Rbm8a | 0.45 | 0.41 | 0.7 | 1.31 |
| Rbms1 | −0.26 | 0 | −0.58 | −1.48 |
| Rbms3 | −0.64 | 0 | −0.59 | −3.37 |
| Rbmx | −0.72 | −0.25 | −0.31 | −1.52 |
| Rbp1 | 0.38 | 1.61 | 1.02 | 1.82 |
| Rcbtb2 | −0.54 | −0.13 | −0.67 | −1.42 |
| Rcc1 | 0.89 | 1.16 | 1.52 | 1.93 |
| Rdbp | 0.43 | 0.37 | 0.47 | 1 |
| Rdx | 0.86 | 1.08 | 1.81 | 1.69 |
| Reep5 | −0.99 | −0.68 | −0.77 | −1.19 |
| Reep6 | −0.84 | −0.78 | −1.03 | −1.71 |
| Rfc2 | 1.06 | 0.57 | 1.12 | 1.62 |
| Rfc3 | 1.45 | 1.71 | 0.81 | 0.11 |
| Rfc4 | 2.03 | 2.37 | 1.68 | 1.64 |
| Rfc5 | 1.64 | 1.54 | 1.65 | 1.25 |
| Rgnef | −0.97 | −0.74 | −0.71 | −1.38 |
| Rgs10 | 1.4 | 0.5 | 0.15 | 0.83 |
| Rgs11 | −0.86 | −0.46 | −1.07 | −1.22 |
| Rgs2 | −2.47 | 0 | −0.98 | 0 |
| Rgs5 | −1.53 | −2.8 | −2.53 | −2.46 |
| Rhoc | −0.22 | 0.03 | 0.66 | 1.53 |
| Rhoq | −1.56 | −1.36 | −0.86 | −1.12 |
| Rhou | −0.04 | 0.12 | 0.6 | 1.29 |
| Rhpn2 | −1.31 | −1.2 | −0.13 | 0.03 |
| Rian | −1.21 | −3.35 | −1.81 | −2.66 |
| Rin1 | −0.19 | −0.19 | −0.57 | −1.04 |
| Ripk3 | 1.68 | 2.32 | 1.45 | 1.5 |
| Rnase4 | −0.22 | −0.09 | −0.57 | −1.08 |
| Rnd3 | 1.73 | 0.96 | 0.11 | 0 |
| Rnf125 | −1.53 | −1.11 | −1.7 | −1.49 |
| Rnf13 | 0.3 | 0.89 | −0.18 | −1.33 |
| Rnf14 | 0.16 | 0.43 | −0.3 | −1.11 |
| Rnf186 | 1.68 | 1.17 | 1.3 | 0 |
| Rnf19 | 0.85 | 1.03 | 0.46 | 0.26 |
| Rnf26 /// | 0.91 | 1.09 | 1.18 | 1.17 |
| LOC668155 /// | | | | |
| LOC669536 | | | | |
| Rnf39 | 0.71 | 1.27 | 1.45 | 1.57 |
| Rnpc2 | 0.93 | 1.42 | 0.24 | −1.03 |
| Rnps1 | 0.61 | 0.42 | 0.75 | 1.12 |
| Rod1 | 1.1 | 1.52 | 1.1 | 0.36 |
| RP23-136K12.4 | −0.12 | 0.06 | −0.73 | −1.13 |
| Rpa1 | 0.9 | 1.16 | 0.87 | 0.69 |
| Rpa2 | 2.23 | 3.31 | 1.94 | 0.93 |
| Rpa3 | 0.89 | 0.82 | 1.1 | 1.6 |
| Rpl37a /// | 0.41 | 1.64 | 0.31 | −0.45 |
| LOC629949 /// | | | | |
| LOC640991 /// | | | | |
| LOC668935 /// | | | | |
| LOC674834 | | | | |
| Rpp25 | 0.81 | 3.32 | 2.3 | 1.67 |
| Rpp30 | 1.22 | 1.04 | 1.04 | 0.92 |
| Rps10 /// | 0.01 | −1.46 | −1.08 | −0.71 |
| LOC621772 /// | | | | |
| LOC636539 /// | | | | |
| LOC666274 /// | | | | |
| LOC668457 | | | | |
| Rps12 /// | −0.18 | −1.01 | −0.82 | −0.84 |
| LOC432865 /// | | | | |
| LOC627835 /// | | | | |
| LOC668354 /// | | | | |
| LOC670751 /// | | | | |
| LOC670832 /// | | | | |
| LOC671641 /// | | | | |
| LOC672008 /// | | | | |
| LOC672415 /// | | | | |
| LOC676277 | | | | |
| Rps24 | −1.05 | −1.03 | −1.08 | −2.81 |
| Rrm1 | 2.25 | 2.64 | 1.83 | 0.35 |
| Rrm2 | 3.85 | 3.41 | 2.79 | 1.49 |
| Rrm2b | −0.31 | −0.04 | −0.59 | −1.98 |
| Rsad1 | −1.06 | −0.85 | −0.73 | −0.95 |
| Rsad2 | 0.15 | 1.19 | 1.02 | 3.07 |
| Rshl3 | −2.92 | −2.78 | −2.44 | −2.72 |
| Rtel1 | 1.05 | 0.72 | 0.9 | 0.62 |
| Rtn4 | 0.3 | −1.07 | −0.33 | −2.24 |
| Rtn4rl1 | −1.12 | −0.62 | −0.58 | −0.72 |
| Rtp4 | 0.32 | 1.97 | 2.8 | 2.67 |
| Rufy3 | −0.5 | 0 | −0.47 | −2.38 |
| Runx1 | 0.11 | 0.25 | −0.21 | −1.83 |
| Rusc1 | −0.83 | −0.12 | −1.17 | −1.15 |
| Ruvbl2 | 1.01 | 0.94 | 0.77 | 0.96 |
| S100a1 | −0.93 | −1.12 | −0.84 | −0.78 |
| S100a10 | 0.84 | 0.5 | 0.8 | 1.1 |
| S100a14 | 1.08 | 1.22 | 1 | 1.37 |
| S100a5 | 0.63 | 0.58 | 1.1 | 1.2 |
| S3-12 | −1.23 | −1.33 | −1.35 | −0.95 |
| Samhd1 | 0.13 | 0.47 | 0.3 | 1.27 |
| Saps3 | −0.15 | −0.11 | −0.31 | −1.19 |
| Sass6 | 1.77 | 2.21 | 1.63 | 1.24 |
| Satb1 | −0.35 | −0.62 | −0.7 | −1.07 |
| Sbk1 | −0.77 | 0.64 | 0 | 1.37 |
| Sbsn | 1.26 | 0.59 | 0.28 | 0.75 |
| Scamp1 | 0.29 | 0.42 | −0.56 | −1.45 |
| Scara3 | 0.46 | 0 | 1.38 | 1.51 |
| Sccpdh | −0.92 | −1.29 | −0.94 | −0.84 |
| Scd2 | 0.47 | 0.67 | −0.41 | −2.34 |
| Sco1 | −0.47 | −0.43 | −0.94 | −1.01 |
| Scoc | −0.19 | 0.66 | −0.69 | −1.24 |
| Scube2 | −2.41 | −2.34 | −1.91 | −2.53 |
| Scyl2 | 0.2 | 1.06 | 0.28 | −0.14 |
| Sdc1 | 0.38 | 0.83 | −0.62 | −2.36 |
| Sdc2 | −2.05 | −1.64 | −1.75 | −1.77 |
| Sdc4 | −0.54 | −1 | −0.1 | 1.08 |
| Sdf4 | −1.31 | −1.4 | −0.68 | −0.79 |
| Sdpr | −1.74 | −1.28 | −1.62 | 0 |
| Sec61a1 | 0.54 | 1.03 | −0.27 | −1.58 |
| Sec63 | 0.07 | 0.13 | −0.39 | −1.23 |
| Seh1l | 0.98 | 1.32 | 0.51 | 0.21 |
| Sele | −0.67 | −0.36 | −0.33 | −1.73 |
| Selk | −0.24 | 0.02 | −0.33 | −1.06 |
| Selm | −1.27 | −1.12 | −0.34 | 0.34 |
| Sema3b | −1.33 | −0.99 | −1.28 | −1.98 |
| Sema3e | 0.32 | −0.23 | −1.01 | −0.36 |
| Sema4c | −1.11 | −1.31 | −1.24 | −1.4 |
| Sema4g | −1.16 | −1.64 | −2.14 | −1.95 |
| Senp8 | −0.15 | −0.24 | −0.31 | −1.04 |
| Sephs1 | 0.84 | 1.29 | 0.71 | 0.08 |
| Serinc1 | 0.02 | 0.32 | −0.64 | −2.6 |
| Serinc3 | 0.04 | 1.11 | 0.47 | −1.17 |
| Serpine1 | 2.76 | 1.83 | 0 | 0.99 |
| Serping1 | −1.65 | −1.89 | −1.86 | −1.17 |
| Serpinh1 | −1.94 | −0.75 | −1.9 | −0.58 |
| Sertad1 | 0.53 | 0.65 | 0.64 | 1.12 |
| Sesn1 | −0.44 | −0.37 | −1.08 | −0.97 |
| Setd5 | 0.51 | 0 | −0.02 | −2.47 |
| Sf1 | 0.31 | 0.11 | 0.74 | 1.08 |
| Sf3a3 | 0.59 | 0.85 | 0.89 | 1.03 |
| Sf3a3 /// | 0.91 | 1.1 | 0.85 | −0.07 |
| LOC626830 | | | | |
| Sf3b3 | 0.6 | −0.03 | −0.01 | −1.5 |
| Sfi1 /// | −0.88 | −0.33 | −0.61 | −1.43 |
| LOC673404 /// | | | | |
| LOC673420 /// | | | | |
| LOC673566 | | | | |
| Sfpq | −0.32 | −1.2 | −0.18 | −1.56 |
| Sfrs1 | 0.96 | 1.56 | 0.26 | −1.38 |
| Sfrs2 | 1.03 | 1.26 | 0.44 | −0.08 |
| Sfrs2ip | −0.09 | −0.29 | 0.37 | 1.28 |
| Sfrs3 | 0.88 | 1.15 | 0.37 | 0.17 |
| Sfrs3 /// | 0.18 | 1.4 | 0.57 | 0.73 |
| LOC632248 | | | | |
| Sfrs4 | 0.53 | 0.61 | 0.71 | 1.13 |
| Sfrs7 | 0.86 | 0.4 | 0.22 | −1.75 |
| Sft2d2 | 1.3 | 1.62 | 1.01 | 2.07 |
| Sgce | 0.6 | 1.22 | 0.45 | 0.47 |
| Sgk2 | 2.09 | 0.94 | 0.88 | 1.18 |
| Sgol1 | 1.62 | 1.58 | 2.97 | 1.97 |
| Sgol2 | 2.91 | 1.8 | 2.34 | 1.89 |
| Sgpl1 | 0.04 | 0.43 | −0.67 | −2.84 |
| Sgpp1 | 1.2 | 1.13 | 0 | −1.1 |
| Sh2bp1 | −0.09 | 0.1 | −0.64 | −1.8 |
| Sh3bgrl | 0.32 | 0.55 | −0.2 | −1.33 |
| Sh3gl2 | −0.57 | −0.06 | −1.18 | −2.92 |
| Sh3gl3 | −2.14 | −3.68 | −2.17 | −4.09 |
| Sh3kbp1 | −0.13 | 0.12 | −0.24 | −1.45 |
| Sh3tc2 | −0.41 | −1.05 | −0.63 | −0.69 |
| Shcbp1 | 5.46 | 2.8 | 4.29 | 2.89 |
| Shfm1 | 0.25 | 0.01 | 0.19 | 1.03 |
| Shh | −0.06 | 0.11 | −0.99 | −1.86 |
| Shmt1 | 0.79 | 3.25 | 0.07 | −1.51 |
| Shoc2 | 0.65 | 1.13 | 0.13 | −0.87 |
| Shrm | −0.15 | −0.13 | 0.36 | 1.24 |
| Siah1a | 0.35 | 0.1 | 0.69 | 1.47 |
| Sidt1 | −0.67 | −0.37 | −0.63 | −1.44 |
| Sip1 | 0.64 | 0.48 | 0.99 | 1.11 |
| Siva | 0.47 | 0.12 | 0.55 | 1.3 |
| Six4 | −0.36 | −0.45 | −1.16 | −1.53 |
| Ski | −0.04 | −0.89 | 0.13 | −1.67 |
| Skp2 | 1.23 | 1.08 | 1.69 | 2.45 |
| Slbp | 1.51 | 1.68 | 1.65 | 1.78 |
| Slc12a2 | −0.04 | 0.44 | 1.04 | 1.47 |
| Slc12a6 | −1.36 | 0 | −0.88 | −3.79 |
| Slc12a7 | −1.04 | −0.28 | −1.04 | −2.07 |
| Slc16a1 | 0.39 | 0.3 | −1.15 | −0.43 |
| Slc16a12 | −1.88 | −1.72 | −1.29 | −1.97 |
| Slc16a3 | 2.61 | 1.59 | 0 | 1.85 |
| Slc16a7 | −0.25 | −0.49 | −0.38 | −1.23 |
| Slc16a9 | −1.38 | −1.79 | −1.33 | −1.02 |
| Slc1a5 | −0.7 | −0.76 | −0.81 | −1.04 |
| Slc22a17 | −0.93 | −1.13 | −0.95 | −1.65 |
| Slc22a18 | −0.67 | −0.77 | −1.87 | −2.57 |
| Slc23a1 | −0.77 | −0.85 | −1.07 | −1.19 |
| Slc23a2 | −0.63 | −0.22 | −0.62 | −1.2 |
| Slc24a6 | −1.01 | −0.16 | −0.42 | −0.22 |
| Slc25a12 | 0.9 | 1.14 | 0.41 | 0.85 |
| Slc25a24 | 0.81 | 1.18 | 0.09 | −1.24 |
| Slc25a35 | −1.07 | −0.92 | −0.92 | −1.43 |
| Slc26a2 | −0.44 | −0.61 | −0.56 | −1.19 |
| Slc29a1 | −0.78 | −0.7 | −0.34 | −1.1 |
| Slc2a1 | 1.81 | 0.72 | 0.36 | 1.69 |
| Slc30a1 | −1.17 | −1.21 | −0.13 | 0.17 |
| Slc35a3 | 0.59 | 1.24 | 0.03 | −0.81 |
| Slc35a5 | −0.52 | −0.68 | −0.75 | −1.1 |
| Slc38a1 | −0.44 | −0.69 | 2.3 | 1.65 |
| Slc39a10 | 0.68 | 0.8 | 0.08 | −1.75 |
| Slc40a1 | 0.63 | 1.06 | 0.16 | −0.46 |
| Slc44a2 | 0.11 | 0.62 | −0.38 | −1.33 |
| Slc44a4 | −1.18 | −0.2 | 0.08 | 0.14 |
| Slc4a4 | −0.72 | −1.3 | −0.28 | 0.27 |
| Slc4a7 | 0.96 | 0 | 0 | −1.5 |
| Slc5a1 | −2.81 | −2.45 | −2.07 | −1.78 |
| Slc6a6 | −0.47 | −1.07 | −0.67 | −3.15 |
| Slc9a2 | −1.02 | −1.07 | −0.36 | −0.34 |
| Slc9a3r2 | −1.26 | 0.1 | 0.48 | 0.46 |
| Slco2a1 | −2.12 | −1.54 | −1.2 | −1.35 |
| Slco3a1 | −2.66 | −2.1 | −1.54 | −4.55 |
| Slfn2 | −0.44 | 0.98 | 0.15 | 1.12 |
| Slfn9 | 1.95 | 1.83 | 1.86 | 1.29 |
| Smarca2 | −1.25 | −1.47 | −1.16 | −1.84 |
| Smarca4 | −0.59 | −1.11 | −0.33 | −0.67 |
| Smarcd3 | 0.79 | 3.13 | 1.36 | 1.6 |
| Smc2l1 | 1.85 | 1.87 | 3.02 | 2.57 |
| Smc4l1 | 1.74 | 0.8 | 2.43 | 0 |
| Smc6l1 | 0.96 | 0.68 | 1.07 | 1.91 |
| Smoc1 | −1.1 | −1.27 | −1.28 | −1.15 |
| Smpdl3b | 1.87 | 1.46 | 2.29 | 2.3 |
| Snai2 | −1.13 | −0.87 | −0.86 | −0.89 |
| Snap23 | 0.17 | 0.83 | −0.47 | −1.35 |
| Snapc3 | −0.57 | 0 | −0.62 | −3.79 |
| Snf1lk2 | 0.42 | 0 | 0.24 | −1.89 |
| Snord22 | 0.95 | 1.35 | 0.58 | 0.3 |
| Snrp1c | 0.53 | 0.83 | 0.83 | 1.04 |
| Snrpa1 | 0.78 | 0.97 | 0.81 | 1.24 |
| Snrpd1 | 0.83 | 0.62 | 0.59 | 1.02 |
| Snrpn | −0.8 | −1.03 | −1.56 | −2.14 |
| Snrpn /// Snurf | −1.09 | −1.34 | −2.71 | −3.59 |
| Snx25 | 0.58 | 0.61 | 0.35 | 1.03 |
| Snx6 | 0.84 | 0.39 | 0.85 | 1.09 |
| Soat1 | −3.57 | −3.14 | −1.84 | −0.03 |
| Socs3 | 0 | 0 | 0 | 1.35 |
| Sod1 | −0.66 | −0.92 | −1.09 | −1.74 |
| Sod3 | −4.45 | −2.67 | −3.09 | −2.51 |
| Solt | 2.99 | 3.14 | 2.92 | 2.68 |
| Son | 0.47 | 0.91 | 0.31 | −1.69 |
| Sorcs2 | −1.06 | −1.05 | −0.73 | −1.3 |
| Sorl1 | −0.5 | 0.06 | −1.03 | −1.34 |
| Sort1 | −1.07 | −1.01 | −1.01 | −1.05 |
| Sox15 | −0.87 | −0.9 | −1.04 | −1.35 |
| Sox4 /// | 0.24 | 0.48 | −0.69 | −4.36 |
| LOC672274 | | | | |
| Spag5 | 2.54 | 2.64 | 3.03 | 2.16 |
| Sparc | −1.45 | −0.78 | −1.33 | 1.49 |
| Sparcl1 | −1.33 | −0.39 | −0.78 | 1.9 |
| Spata6 | −3.02 | −1.19 | −1.05 | −0.72 |
| Spbc24 | 4.32 | 4.5 | 1.93 | 2.23 |
| Spbc25 | 2.53 | 2.32 | 2.56 | 2.64 |
| Sphk1 | −1.72 | −1.44 | −1.3 | −0.64 |
| Spin | 0.43 | 1.08 | 0.13 | 0.2 |
| Spire2 | 0.02 | 0.35 | 0.52 | 1.04 |
| Spnb2 | −0.34 | −1.05 | −0.34 | 0.54 |
| Spon1 | −2.09 | −2.52 | −1 | −1.86 |
| Spop | 0.02 | 0.35 | −0.73 | −1.5 |
| Spp1 | 0 | 0 | 0 | 3.15 |
| Sprr2f | 3.67 | 3.83 | 3.3 | 3.88 |
| Sprr2h | 0 | 0 | 0 | 3.28 |
| Spry2 | −0.48 | −0.84 | −1.09 | −0.98 |
| Sptlc1 | −0.12 | 0.95 | −1.34 | −0.66 |
| Srd5a1 | −0.54 | −0.42 | −1.3 | −1.73 |
| Sri | −0.14 | −0.09 | −0.18 | −1.1 |
| Srprb | 0.66 | 1.19 | 0.35 | −0.97 |
| Srpx2 | −0.62 | −0.56 | −1.27 | −1.65 |
| Srrm2 | −0.39 | −0.7 | −0.36 | −1.99 |
| Ssh1 | −1.15 | −0.58 | −0.23 | 0.44 |
| Ssrp1 | 0.74 | 0.55 | 0.77 | 1.04 |
| St14 | 0.09 | 0.15 | 0.51 | 1.09 |
| Stag1 | 0.94 | 1.15 | 1.09 | −0.52 |
| Stag2 | 0.86 | 1.11 | −0.01 | −0.46 |
| Stam2 | 0.52 | 1.34 | −0.03 | −0.4 |
| Stambp | 0.07 | 0.5 | −0.46 | −1.39 |
| Stard13 | −0.48 | −0.76 | −0.72 | −1.09 |
| Stch | 1.08 | 0.64 | −0.04 | −1.05 |
| Stfa3 | 1.08 | 0 | 2.09 | 3.42 |
| Stmn1 /// | 0.86 | 1.21 | 1.57 | 1.27 |
| LOC639458 | | | | |
| Stoml1 | −0.62 | −0.97 | −1.15 | −1.32 |
| Stox2 | −0.59 | −0.6 | −1.74 | −0.87 |
| Sub1 | −0.61 | −0.37 | −1.27 | −1.22 |
| Sulf1 | −1.2 | −1.81 | −1.39 | −2.57 |
| Suox | −1.34 | −1.04 | −1.05 | −1.17 |
| Supt16h | 1.15 | 1.74 | 0.39 | −1 |
| Susd4 | −0.93 | −1.18 | −0.34 | 0.05 |
| Suv39h1 | 1.33 | 1.59 | 1.67 | 1.47 |
| Syce2 | 3.1 | 2.84 | 2.29 | 2.82 |
| Synj2bp | −1.29 | 0 | 0 | −3.41 |
| Sypl | 0.26 | 0.61 | −0.06 | −1.44 |
| Sytl2 | −0.68 | −0.65 | −0.76 | −1.34 |
| Sytl3 | −1.07 | −0.03 | −0.97 | −1.03 |
| Tacc3 | 2.94 | 2.08 | 2.92 | 2.32 |
| Taf15 | 0.38 | 0 | 0.21 | −1.8 |
| Tagln | −2.73 | −1.55 | −1.89 | 0 |
| Tagln2 | 1.23 | 1 | 0.99 | 1.04 |
| Tagln2 /// | 1.71 | 1.17 | 1.57 | 1.9 |
| LOC672466 | | | | |
| Tanc2 | −0.6 | −1.04 | −0.66 | 0.27 |
| Tardbp | 0.73 | 1.12 | 0.33 | −0.43 |
| Tbc1d10b | 0.17 | 0.26 | 0.47 | 1.27 |
| Tbc1d8 | −0.21 | 0.18 | −0.38 | −1.62 |
| Tbc1d9 | −1.18 | −1.63 | −1.23 | −1.95 |
| Tbx1 | −1.64 | −1.45 | 0.13 | −0.43 |
| Tbx3 | −0.9 | −0.95 | −0.82 | −1.42 |
| Tcea1 | 0.77 | 1.17 | 0.45 | −0.33 |
| Tceal8 | 0.05 | −0.11 | −0.6 | −1.14 |
| Tcf12 | 0.34 | 0 | 0.03 | −3.46 |
| Tcf19 | 2.84 | 2.81 | 2.78 | 2.56 |
| Tcf21 | −2.27 | −1.59 | −1.26 | 0 |
| Tcf25 | −0.36 | −0.83 | −0.6 | −1.26 |
| Tcf4 | −0.62 | −1.2 | −0.19 | −0.87 |
| Tcfap2a | 0.93 | 2.11 | 1.34 | 1.44 |
| Tcfap2c | 0.21 | 0.63 | 0.67 | 1.18 |
| Tcfcp2l1 | −1.04 | −0.59 | −0.05 | 0.39 |
| Tcn2 | −0.84 | −0.64 | −1.15 | −1.59 |
| Tcof1 | 0.25 | −0.11 | 0.75 | 1.23 |
| Tcp11l2 | −0.62 | −0.64 | −0.71 | −1 |
| Tcta | −0.93 | −1.17 | −1.04 | −1.48 |
| Tdrkh | 0 | 0.63 | 1.11 | 0.87 |
| Tead2 | 0.29 | 0.44 | 0.72 | 1.08 |
| Tec | 0 | 0.12 | 1.29 | 1.25 |
| Terf1 | 1.06 | 1.32 | 1.14 | 0.84 |
| Tex19 | 0 | 0 | 1.74 | 2.15 |
| Tff1 | 0.81 | 0.95 | 1.13 | 0.92 |
| Tfrc | 1.72 | 1.63 | 0.76 | −0.23 |
| Tgfbi | −1.74 | −1.93 | −1.46 | −0.36 |
| Tgm2 | −1.73 | −1.01 | −0.29 | 0.49 |
| Tgoln1 | −0.23 | 0.18 | −0.41 | −2.14 |
| Th1l | 0.63 | 0.35 | 0.47 | 1.03 |
| Thbs1 /// | 0.03 | 0.07 | −0.25 | −1.37 |
| LOC640441 | | | | |
| Thbs2 | −4.12 | −2.01 | 0 | 0 |
| Thex1 | 1.17 | 0.85 | 0.85 | 1.02 |
| Thoc4 | 1.1 | 1 | 0.8 | 1.24 |
| Thoc4 /// | 0.94 | 1.16 | 0.86 | 1.31 |
| Refbp2 | | | | |
| Thrap3 | 0.21 | 0.91 | 0.01 | −1.04 |
| Timeless | 2.85 | 3.01 | 2.15 | 2.28 |
| Timm50 | 1.19 | 0.69 | 0.81 | 0.91 |
| Timm8a1 | 1.2 | 0.92 | 0.57 | 0.58 |
| Timp2 | −1.69 | −1.2 | −1.25 | 0 |
| Tinagl | 0.97 | 0.69 | 1.21 | 1.56 |
| Tinf2 | 0.85 | 0.74 | 1.78 | 0.85 |
| Tiparp | −0.94 | −1.15 | −0.57 | 0.4 |
| Tipin | 1.84 | 2.08 | 1.84 | 1.85 |
| Tk1 | 2.55 | 2.56 | 2.73 | 1.48 |
| Tk2 | −1.05 | −1.54 | −1.38 | −1.41 |
| Tlr2 | −0.17 | 0.37 | 1.03 | 1.33 |
| Tm7sf2 | −0.8 | −0.89 | −1.51 | −1 |
| Tmcc3 | 0.11 | 0.11 | −0.27 | −1.38 |
| Tmed2 | 0.85 | 1.43 | −0.11 | −1.61 |
| Tmem134 | 0.13 | 0.25 | −0.39 | −1.51 |
| Tmem141 | −0.72 | −0.62 | −0.41 | −1.11 |
| Tmem146 | −1.05 | −0.73 | −0.99 | −1.1 |
| Tmem14a | −0.67 | −1.24 | −0.77 | −0.86 |
| Tmem16a | −0.22 | −0.83 | −1.17 | −1.84 |
| Tmem32 | 0.6 | 1.35 | 0.2 | −0.81 |
| Tmem33 | −0.01 | 0.12 | −0.51 | −1.46 |
| Tmem34 | −0.71 | −0.86 | −0.76 | −1.48 |
| Tmem45a | 0 | 0 | 2.36 | 4.83 |
| Tmem48 | 1.88 | 1.93 | 2.27 | 1.15 |
| Tmem49 | 0.7 | 0.67 | −0.07 | −1.07 |
| Tmem57 | −0.54 | −1.1 | −0.61 | −2.26 |
| Tmem58 | −1.42 | −1.81 | −1.44 | −0.45 |
| Tmem64 | −1.05 | −0.82 | −0.77 | −1.31 |
| Tmem71 | 1.37 | 0.8 | 1.14 | 0.97 |
| Tmem8 | −1.73 | −2.13 | −0.94 | −1.37 |
| Tmepai | −1.12 | −1.21 | −0.63 | −0.79 |
| Tmie | 0 | 0.43 | 2.74 | 1.4 |
| Tmpo | 1.21 | 1.63 | 1.38 | 0.49 |
| Tmprss11a | −0.58 | 0.04 | −0.98 | −1.72 |
| Tmprss2 | −0.22 | −0.06 | −0.66 | −1.42 |
| Tnc | −2.9 | −2.62 | −0.74 | 0 |
| Tnfaip8l1 | 0.75 | 1.11 | 1.8 | 1.61 |
| Tnfrsf12a | 2.8 | 1.85 | 1.84 | 2.64 |
| Tnfsf5ip1 | 0.79 | 1.15 | 0.44 | 0.2 |
| Tnfsf9 | 2.15 | 1.64 | 2.63 | 3.8 |
| Tnks1bp1 | 0.01 | −0.12 | 0.17 | 1.01 |
| Tnni2 | 0.82 | 1.03 | 0.52 | 0.89 |
| Tnpo1 | −0.03 | 0 | −0.41 | −1.44 |
| Tnpo2 | 0.54 | 1.02 | 0.36 | −0.66 |
| Tnrc9 | −2.16 | −1.61 | −0.44 | −2.6 |
| Tns1 | −1.18 | −1.44 | −1.12 | −0.03 |
| Tns4 | −0.19 | −0.57 | −0.72 | −1.33 |
| Tob2 | 0.41 | 0.38 | 1.41 | 1.97 |
| Toe1 | 0.74 | 1.81 | 0.76 | −0.33 |
| Tomm20 | −0.81 | −0.81 | −0.82 | −1.03 |
| Top2a | 3.8 | 3.4 | 4.27 | 2.57 |
| Top2b | 0.52 | 0.64 | 0.3 | −1.09 |
| Topbp1 | 1.65 | 1.56 | 1.55 | 1.38 |
| Topors | 0.11 | 0.44 | 1.01 | 1.2 |
| Tpi1 | 0.85 | 0.76 | 0.53 | 1.26 |
| Tpm2 | −1.03 | −0.52 | −1.19 | 1.36 |
| Tpmt | −0.02 | −0.19 | −0.7 | −1.2 |
| Tpp1 | −0.62 | −1.06 | −0.88 | −1.12 |
| Tpx2 | 2.28 | 2.08 | 2.24 | 2.41 |
| Traip | 2.07 | 2.32 | 3.1 | 1.67 |
| Trak1 | −0.54 | −0.54 | 0.64 | 1.24 |
| Trf | −0.45 | −0.62 | −1.42 | −2.81 |
| Trim11 | −0.46 | −0.25 | −0.53 | −1.02 |
| Trim2 | 1.26 | 1.23 | 3.5 | 1.18 |
| Trim25 | 1.24 | 1.2 | 0.26 | 0.96 |
| Trim27 | 1.4 | 0.34 | 0.12 | 0.56 |
| Trim37 | 0.56 | 0.54 | 0.76 | 1.44 |
| Trim59 /// | 2.5 | 5.12 | 2.6 | 0 |
| LOC630539 | | | | |
| Trim68 | −0.59 | −0.73 | −0.63 | −1.03 |
| Trio | −0.52 | 0 | −0.51 | −1.27 |
| Trip13 | 2.13 | 1.32 | 1.91 | 2.1 |
| Trp53 | 0.75 | 1.32 | 0.2 | 0 |
| Trp53i11 | −0.56 | −1.26 | −1.1 | −1.42 |
| Trp53i5 | 1.13 | 1.32 | 1.37 | 1.39 |
| Trp53inp2 | −1.27 | −0.99 | −0.43 | −0.44 |
| Trp63 | −0.12 | −1.56 | −1.37 | −5.32 |
| Trpv4 | −0.64 | −0.84 | −0.73 | −1.29 |
| Tsc22d3 | −1 | −0.22 | −0.67 | −1.13 |
| Tspan1 | 0 | 0 | 2.55 | 5.16 |
| Tspan17 | −1.13 | −1.26 | −1.06 | −1.1 |
| Tspan33 | 0.88 | 1.61 | −0.05 | 1.49 |
| Tspan6 | −0.45 | −0.59 | −0.75 | −1.08 |
| Tspan7 | 0.6 | 1.13 | −0.17 | −0.92 |
| Tspan8 | 0.32 | 1.09 | 1.13 | 1.57 |
| Tssc4 | 0.92 | 0.78 | 0.98 | 1.11 |
| Ttc1 | 0.33 | 0.26 | −0.49 | −1.65 |
| Ttc12 /// | −0.74 | −1.24 | −1.13 | −1.32 |
| LOC640389 | | | | |
| Ttc14 | −0.46 | −0.47 | −0.43 | −1.25 |
| Ttc9c | 0.94 | 1.53 | 0.06 | −0.29 |
| Ttk | 3.57 | 2.93 | 3.21 | 2.86 |
| Ttr | −2.84 | −3.74 | −4.18 | −3.86 |
| Tuba1 | −0.29 | −0.13 | 0.38 | 1.07 |
| Tuba3 /// Tuba7 | 0.78 | 3.85 | 0.97 | 0.49 |
| /// LOC384954 | | | | |
| Tubb5 | 0.52 | −0.26 | 0.88 | 1.1 |
| Tug1 | 0.31 | 1.15 | −0.72 | −2.54 |
| Twsg1 | 0.7 | 1.02 | 0.05 | −1.52 |
| Txlna | 0.76 | 0.91 | 1.04 | 0.21 |
| Txndc13 | −1.17 | −0.86 | −0.8 | −0.3 |
| Txnip | 0.01 | 0.69 | −0.59 | −1.72 |
| Tyms /// Tyms- | 1.54 | 1.79 | 1.67 | 1.34 |
| ps | | | | |
| Tyro3 | −0.53 | −0.41 | −0.72 | −1.51 |
| U2af1-rs1 | −0.69 | −0.48 | −0.68 | −1.16 |
| Ubap2l | 0.41 | 0.47 | 0.64 | 1.09 |
| Ube2b | −0.3 | −1.02 | −0.6 | −1.62 |
| Ube2c | 4.42 | 4.34 | 5.13 | 2.96 |
| Ube2g2 | 0.34 | 1.2 | −0.13 | −2.6 |
| Ube2h | −0.47 | −0.76 | −0.51 | −2.03 |
| Ube2s | 1.05 | 1.07 | 0.83 | 0.79 |
| Ube2t | 2.11 | 2.2 | 2.18 | 2.18 |
| Ube4b | 0.17 | 1.24 | 0.15 | −0.16 |
| Ubtf | −1.09 | −0.79 | 1.23 | 2.24 |
| Uchl1 | −1.86 | −2.01 | −2.49 | −1.78 |
| Uchl3 | 0.39 | 0 | 0.36 | −1.71 |
| Ugcg | 0.9 | 1.37 | 0.6 | −0.14 |
| Ugdh | −0.65 | −0.99 | −1.22 | −2 |
| Ugt1a2 /// | −0.37 | −0.42 | −0.89 | −1.78 |
| Ugt1a6a /// | | | | |
| Ugt1a10 /// | | | | |
| Ugt1a7c /// | | | | |
| Ugt1a5 /// | | | | |
| Ugt1a9 /// | | | | |
| Ugt1a6b /// | | | | |
| Ugt1a1 | | | | |
| Ugt2b34 | −0.59 | −0.73 | −1.18 | −2.48 |
| Uhrf1 | 5.21 | 3.05 | 2.85 | 1.7 |
| Unc13b | 0 | 0 | 1.6 | 1.67 |
| Unc5a | −0.68 | −1.11 | −0.79 | −1.86 |
| Unc84a | −0.05 | 0.3 | −0.21 | −1.25 |
| Unc93b1 | −1 | −0.61 | −0.68 | −0.64 |
| Ung | 2.26 | 2.15 | 2.29 | 3.43 |
| Upk1b | −0.55 | −0.78 | −0.86 | −1.46 |
| Upk2 | −0.99 | −1.03 | −0.62 | −0.85 |
| Usp1 | 1.64 | 2.37 | 1.52 | 0.26 |
| Usp15 | 0.46 | 0.88 | 0.05 | −1.11 |
| Usp27x | −0.92 | −0.77 | −1.18 | −1.19 |
| Usp32 | 0.11 | 0 | −0.43 | −1.53 |
| Usp37 | 0.36 | 0.76 | 0.91 | 1.52 |
| Usp6nl | 0.22 | 0.37 | 0.71 | 1.72 |
| Utx | 0.58 | 0 | −0.08 | −1.58 |
| Vamp3 | 1.17 | 1.74 | 0.75 | 0.24 |
| Vamp4 | −0.86 | −1.1 | −0.66 | −0.74 |
| Vapb | −0.9 | −0.75 | −0.78 | −1.03 |
| Vasn | −0.31 | 0.38 | 0.14 | 1.28 |
| Vav3 | 1.33 | 1.7 | 0.55 | −0.74 |
| Vegfa | 2.07 | 1.32 | 0 | 0.36 |
| Vgll1 | 0 | 1.9 | 1.84 | 0 |
| Vil2 | 0.14 | 0.11 | 0.41 | 1.06 |
| Vim | −1.16 | −1.32 | −1.81 | 0.6 |
| Vldlr | −1.03 | −2.18 | −1.25 | −1.36 |
| Vnn1 | 0.87 | 1.36 | 1.24 | 0.69 |
| Vps25 | 0.3 | 0.32 | −0.43 | −1.09 |
| Vps26b | 0.41 | 0.37 | −0.19 | −1.28 |
| Vps35 | 1.11 | 1.78 | −0.3 | −1.62 |
| Vps41 | 0 | 0.22 | −0.56 | −1.91 |
| Vrk1 | 1.55 | 1.5 | 1.66 | 1.72 |
| W91776 | 0.62 | 0 | 0.35 | −1.23 |
| Wac | 0.26 | −0.6 | 0.02 | −1.89 |
| Wbp5 | 0.5 | 0.51 | 0.8 | 1.32 |
| Wbscr27 | −1.23 | −1.51 | −1.53 | −1.29 |
| Wdfy3 | −0.65 | −1.05 | −0.49 | −0.51 |
| Wdhd1 | 1.95 | 2.22 | 1.9 | 1.84 |
| Wdr1 | 0.59 | 1.05 | −0.24 | −1.7 |
| Wdr23 | −0.51 | −0.46 | −0.54 | −1.15 |
| Wdr40b | −1.23 | −1.52 | −0.94 | −0.88 |
| Wdr5 | 0.95 | 1.09 | 0.7 | 0.66 |
| Wdr57 | 0.65 | 0.39 | 0.59 | 1.66 |
| Wdr76 | 1.12 | 1.1 | 1.05 | 1.27 |
| Wdr77 | 0.72 | 1.01 | 0.13 | −0.5 |
| Wdr8 | 0.21 | 0.15 | 1.04 | 0.85 |
| Wee1 | 1.32 | 1.81 | 0.9 | −0.58 |
| Wfdc15 | −1.78 | −1.74 | −2.24 | −2.79 |
| Whsc1 | 0.88 | 1.52 | 1.56 | 2.13 |
| Wig1 | −0.93 | −0.8 | −0.95 | −1.24 |
| Wiz | 2.01 | 1.59 | 1.28 | 0 |
| Wnk1 | −0.54 | −1.07 | −0.09 | 0.94 |
| Wnt5a | −1.27 | −1.16 | −1.69 | −2.05 |
| Wwtr1 | 0.39 | 0.16 | 0.6 | 1.37 |
| Xist | 1.27 | 2.37 | 0.11 | −4.66 |
| Xlr3a /// Xlr3b | −1.39 | −0.48 | −0.5 | 0.67 |
| /// MGC76689 | | | | |
| Yif1a | 0.19 | −1.36 | −0.4 | 0.03 |
| Yipf2 | −1 | −1.13 | −0.74 | −0.28 |
| Ykt6 | 0.82 | 1.05 | −0.18 | −0.8 |
| Ypel2 | −0.68 | 0 | −1.26 | −1.86 |
| Ypel5 | −0.63 | −0.87 | −0.67 | −1.2 |
| Ywhah | 0.71 | 0.92 | 0.86 | 1.08 |
| Za20d2 | 0.74 | 1.18 | 0.13 | −0.08 |
| Za20d3 | 0.29 | 0.64 | −0.09 | −1.26 |
| Zadh1 | −0.13 | −0.03 | −0.53 | −1.25 |
| Zbtb20 | −0.98 | −1.84 | −0.63 | −1.17 |
| Zbtb4 | −1.34 | −1.59 | −1.55 | −0.94 |
| Zcchc7 | −0.13 | 0 | −0.43 | −2.59 |
| Zdhhc14 | −0.19 | 0.43 | −0.52 | −1.31 |
| Zdhhc18 | 0.04 | 0.42 | 0.88 | 1.03 |
| Zdhhc9 | −0.91 | −1.14 | −1.15 | −0.78 |
| Zfand3 | −0.16 | −0.02 | 0.46 | 1.2 |
| Zfml | 0.42 | 0.79 | 0.06 | −1.48 |
| Zfp101 | 1.14 | 0.95 | 0.96 | 0.07 |
| Zfp185 | 1.73 | 1.82 | 1.77 | 0.75 |
| Zfp192 | −0.29 | −0.65 | −0.56 | −1.14 |
| Zfp207 | −0.21 | 0.32 | −0.67 | −2.8 |
| Zfp260 /// | 0.43 | 0.87 | −0.43 | −1.7 |
| LOC635007 | | | | |
| Zfp313 | 0.22 | 0.79 | −0.26 | −1.06 |
| Zfp367 | 1.1 | 1.13 | 1.22 | 1.28 |
| Zfp369 | −0.3 | −0.64 | −0.38 | −1.08 |
| Zfp445 | 0.37 | 0.52 | −0.33 | −1.82 |
| Zfp458 | −1.15 | −0.56 | 0.18 | −0.6 |
| Zfp462 | −3.32 | −4.55 | −2.46 | −2.95 |
| Zfp467 | −0.93 | −1.19 | −0.92 | −0.94 |
| Zfp608 | −0.12 | 0 | −0.39 | −1.72 |
| Zfp62 | 0.23 | 0.43 | −0.25 | −1.49 |
| Zfp622 | −0.35 | −1.19 | −0.26 | 0.34 |
| Zfp655 | 1.02 | 1.06 | 0.37 | −0.66 |
| Zfp91 | 0.4 | 0.11 | 0.67 | 1.84 |
| Zfpn1a2 | 0.02 | −0.41 | 0.02 | −2.02 |
| Zfpn1a3 | −0.61 | −0.23 | −0.07 | −1.06 |
| Zfyve27 | −0.84 | −1.16 | −0.42 | −0.84 |
| Zmat5 | 0 | −2.86 | −2.45 | −2.39 |
| Zmym1 | 0.77 | 0.71 | 1.15 | 1.01 |
| Zmym6 | −0.55 | −0.27 | −0.15 | −1.01 |
| Zmynd19 | 1.39 | 1.19 | 1.2 | 1.49 |
| Znhit3 | 0.91 | 1.48 | 0.5 | 0.21 |
| Zranb3 | 1.09 | 1.11 | 1.19 | 1.29 |

TABLE 2

| | 3 Wk | | | 6 Wk | | | 20 Wk | | | 30 Wk | | |
|---|---|---|---|---|---|---|---|---|---|---|---|---|
| Top Functions | Score | Focus Mol | Up/Down | Score | Focus Mol | Up/Down | Score | Focus Mol | Up/Down | Score | Focus Mol | Up/Down |
| DRRR, CC, C | 43 | 35/35 | 32/3 | 43 | 35/35 | 33/2 | 43 | 35/35 | 32/3 | 43 | 35/35 | 33/2 |
| CM, CAO, HSDF | 40 | 34/35 | 16/18 | 40 | 34/35 | 14/19 | 40 | 34/35 | 11/21 | 40 | 34/35 | 10/22 |
| CC, CM, CAO | 40 | 34/35 | 24/10 | 40 | 34/35 | 22/9 | 40 | 34/35 | 24/10 | 40 | 34/35 | 24/10 |
| C, GID, GD | 40 | 34/35 | 23/11 | 40 | 34/35 | 25/9 | 40 | 34/35 | 18/16 | 40 | 34/35 | 13/20 |
| RPTM, CAO, CGP | 40 | 34/35 | 22/12 | 40 | 34/35 | 22/10 | 40 | 34/35 | 22/12 | 40 | 34/35 | 17/16 |
| DRRR, GE, OD | 40 | 34/35 | 22/9 | 40 | 34/35 | 29/5 | 40 | 34/35 | 21/13 | 40 | 34/35 | 20/14 |
| GE, DRRR, CAO | 38 | 33/35 | 25/8 | 38 | 34/35 | 24/9 | 38 | 33/35 | 24/9 | 38 | 33/35 | 22/11 |
| RPTM, CAO, ESDF | 38 | 33/35 | 25/7 | 38 | 33/35 | 24/5 | 38 | 33/35 | 25/8 | 38 | 33/35 | 15/18 |
| AAM, SMB, C | 35 | 32/35 | 24/8 | 35 | 32/35 | 25/7 | 35 | 32/35 | 19/13 | 35 | 32/35 | 17/15 |
| DRRR, CC, CAO | 35 | 32/35 | 27/5 | 35 | 32/35 | 26/5 | 35 | 32/35 | 25/7 | 35 | 32/35 | 21/9 |
| CMB, CD, CSDF | 35 | 32/35 | 23/8 | 35 | 32/35 | 20/9 | 35 | 32/35 | 19/13 | 35 | 32/35 | 21/10 |
| CAO, CFM, SMSDF | 33 | 31/35 | 18/12 | 33 | 31/35 | 19/10 | 33 | 31/35 | 13/18 | 33 | 31/35 | 8/23 |
| DRRR, C, RUD | 33 | 31/35 | 23/8 | 33 | 31/35 | 23/7 | 33 | 31/35 | 21/8 | 33 | 31/35 | 22/8 |
| CC, C, CMP | 33 | 31/35 | 22/9 | 33 | 31/35 | 22/9 | 33 | 31/35 | 22/9 | 33 | 31/35 | 20/8 |
| CMB, SMB, C | 33 | 31/35 | 19/12 | 33 | 31/35 | 19/10 | 33 | 31/35 | 15/15 | 33 | 31/35 | 12/18 |
| CGP, CC, C | 33 | 31/35 | 19/12 | 33 | 31/35 | 19/12 | 33 | 31/35 | 17/14 | 33 | 31/35 | 17/14 |
| C, GID, RSD | 31 | 30/35 | 10/19 | 31 | 30/35 | 12/17 | 31 | 30/35 | 7/22 | 31 | 30/35 | 5/25 |
| CAO, DRRR, AAM | 31 | 30/35 | 22/8 | 31 | 30/35 | 23/7 | 31 | 30/35 | 20/9 | 31 | 30/35 | 15/14 |
| GE, CAO, AAM | 29 | 29/35 | 14/14 | 29 | 29/35 | 14/13 | 29 | 29/35 | 9/19 | 29 | 29/35 | 7/21 |
| DM, MT, SMB | 29 | 29/35 | 19/9 | 29 | 29/35 | 20/8 | 29 | 29/35 | 15/13 | 29 | 29/35 | 14/13 |
| C, CTCSI, RSD | 27 | 29/35 | 7/22 | 27 | 29/35 | 9/18 | 27 | 29/35 | 6/22 | 27 | 29/35 | 9/18 |
| CI, CVD, AAM | 26 | 27/35 | 9/17 | 26 | 27/35 | 10/16 | 26 | 27/35 | 10/17 | 26 | 27/35 | 10/15 |
| CCP, PD, CSDF | 26 | 27/35 | 10/17 | 26 | 27/35 | 10/15 | 26 | 27/35 | 3/23 | 26 | 27/35 | 2/24 |
| GE, CD, C | 26 | 27/35 | 21/6 | 26 | 27/35 | 22/4 | 26 | 27/35 | 21/6 | 26 | 27/35 | 20/6 |
| C, CC, ESD | 26 | 27/35 | 20/7 | 26 | 27/35 | 19/6 | 26 | 27/35 | 20/6 | 26 | 27/35 | 20/7 |
| CAO, ID, ND | 25 | 28/35 | 7/21 | 25 | 28/35 | 9/16 | 25 | 28/35 | 7/19 | 25 | 28/35 | 6/19 |
| CS, CAO, CMP | 23 | 25/35 | 9/15 | 23 | 25/35 | 12/12 | 23 | 25/35 | 9/16 | 23 | 25/35 | 8/15 |
| CTCSI, HSDF, ILSDF | 23 | 25/35 | 13/12 | 23 | 25/35 | 14/10 | 23 | 25/35 | 12/13 | 23 | 25/35 | 11/13 |
| CD, CGP, IR | 23 | 25/35 | 2/21 | 23 | 25/35 | 1/21 | 23 | 25/35 | 4/19 | 23 | 25/35 | 7/17 |
| LM, SMB, C | 21 | 24/35 | 9/13 | 21 | 24/35 | 9/13 | 21 | 24/35 | 9/15 | 21 | 24/35 | 12/12 |
| GE, CC, C | 21 | 24/35 | 18/6 | | | | 20 | 23/35 | 16/7 | | | |
| CVD, HD, CAO | 20 | 23/35 | 10/12 | 20 | 23/35 | 11/12 | 20 | 23/35 | 8/14 | 20 | 23/35 | 8/13 |
| LM, MD, NAM | 20 | 23/35 | 9/13 | 20 | 23/35 | 12/11 | 20 | 23/35 | 10/12 | 20 | 23/35 | 10/13 |
| LM, SMB, C | 20 | 23/35 | 14/9 | 20 | 23/35 | 12/11 | | | | | | |
| GE, CC, VF | | | | 20 | 23/35 | 16/6 | | | | | | |
| TM, CSDF, CDT | 18 | 22/35 | 11/10 | | | | | | | | | |
| CFM, CGP, HSDF | 18 | 22/35 | 10/12 | | | | | | | 17 | 21/35 | 1/20 |
| AAM, PTM, SMB | 18 | 22/35 | 13/9 | 17 | 21/35 | 10/8 | 17 | 21/35 | 13/8 | 18 | 22/35 | 9/13 |
| AD, CVD, GD | 18 | 22/35 | 7/15 | | | | 17 | 21/35 | 5/15 | 18 | 22/35 | 5/17 |
| LM, SMB, MT | | | | | | | 18 | 22/35 | 10/12 | 17 | 21/35 | 7/14 |
| CS, CMB, DM | | | | | | | 18 | 22/35 | 11/11 | | | |
| C, CCP, RPD | | | | 15 | 20/35 | 11/9 | 17 | 21/35 | 12/9 | 18 | 22/35 | 14/6 |
| CVD, TM, CMP | 17 | 21/35 | 10/11 | 18 | 22/35 | 11/11 | 15 | 20/35 | 10/10 | | | |
| CS, CD, CTDF | 17 | 21/35 | 9/11 | 15 | 20/35 | 10/9 | | | | | | |
| GE, ESD, GD | 17 | 16/19 | 5/11 | 17 | 16/19 | 5/11 | 17 | 16/19 | 3/13 | 17 | 16/19 | 3/13 |
| DRRR, NAM, SMB | | | | 17 | 21/35 | 10/7 | 15 | 20/35 | 8/12 | 17 | 21/35 | 7/13 |
| C, GID, NTD | | | | 17 | 21/35 | 5/15 | | | | | | |
| GD, SMD, CSDF | 16 | 21/35 | 8/12 | | | | | | | | | |
| CC, C, GE | 16 | 20/34 | 12/7 | | | | 16 | 20/34 | 11/9 | | | |
| CD, LM, MT | 16 | 15/18 | 10/4 | 16 | 15/18 | 10/5 | 16 | 15/18 | 9/6 | 16 | 15/18 | 7/8 |
| CC, CTDF, PTM | | | | 15 | 20/35 | 9/8 | 16 | 21/35 | 11/10 | | | |
| AAM, PTM, SMB | 15 | 20/35 | 12/8 | 15 | 20/35 | 14/4 | 15 | 20/35 | 10/9 | 15 | 20/35 | 10/10 |
| LM, MT, SMB | | | | | | | 15 | 20/35 | 11/9 | | | |
| CTCSI, CAO, CDT | | | | | | | | | | 15 | 20/35 | 9/11 |
| PTM, PF, RSDF | | | | | | | | | | 15 | 20/35 | 4/15 |
| CSDF, CD, CGP | | | | | | | | | | 15 | 20/35 | 9/11 |

Legend
C Cancer
CAO Cellular Assembly and Organization
GE Gene Expression
CC Cell Cycle
DRRR DNA Replication, Recombination, and Repair
AAM Amino Acid Metabolism
CD Cellular Development
LM Lipid Metabolism
SMB Small Molecule Biochemistry
CVD Cardiovascular Disease
CGP Cellular Growth and Proliferation
CM Cellular Movement
TM Tissue Morphology
CMB Carbohydrate Metabolism
CS Cell Signaling
GD Genetic Disorder
RPTM RNA Post-Transcriptional Modification
CFM Cellular Function and Maintenance
CSDF Cardiovascular System Development and Function
CTCSI Cell-To-Cell Signaling and Interaction
CMP Cell Morphology
GID Gastrointestinal Disease
PTM Post-Translational Modification
AD Auditory Disease
CCP Cellular Compromise
CI Cardiac Infarction
DM Drug Metabolism
ESD Endocrine System Disorders
HSDF Hematological System Development and Function
MT Molecular Transport
HD Hematological Disease
ID Inflammatory Disease
MD Metabolic Disease
PD Protein Degradation
RSD Reproductive System Disease
SMD Skeletal and Muscular Disorders
CDT Cell Death
CTDF Connective Tissue Development and Function
ESDF Endocrine System Development and Function
HSDF Hair and Skin Development and Function
ILSDF Immune and Lymphatic System Development and Function
IR Immune Response
NAM Nucleic Acid Metabolism
ND Neurological Disease
OD Organ Development
RUD Renal and Urological Disease
SMSDF Skeletal and Muscular System Development and Function
VF Viral Function
NTD Nutritional Disease
RPD Respiratory Disease
PF Protein Folding
RSDF Respiratory System Development and Function

TABLE 3

| | | |
|---|---|---|
| AASDHPPT | GGA2 | POLA1 |
| AATF | GINS4 | POLB |
| ABCA1* | GIPC2 | POLD1 |
| ABCA3* | GJB6* | POLD2 |
| ABCC3* | GLDC | POLG |
| ABHD6 | GLRX | PON3* |
| ABHD8 | GLS | POSTN* |
| ABHD12 | GMCL1 | PPAP2B* |
| ABHD14B | GMFB | PPARA |
| ACAA2 | GMNN | PPFIBP2 |
| ACER2 | GNE | PPM1B |
| ACLY | GNG10* | PPM1H |
| ACOX1 | GNS | PPP1R8 |
| ACTG2 (includes EG: 72) | GOLIM4 | PPP1R12A |
| ADAM10* | GOLPH3 | PRDM4 |
| ADAM28* | GOLPH3L | PRDX4 |
| ADAMDEC1* | GORASP2 | PRIM1 |
| ADH7 | GOT1 | PRIM2 |
| ADI1 | GOT2 | PRKAR2A |
| ADK | GPBP1 | PRKAR2B |
| AEBP1 | GPC4* | PRKCH |
| AGPAT1 | GPLD1 | PRKCI |
| AGPS | GPM6A* | PROCR* |
| AHCYL1 | GPR137B* | PRPF4B |
| AHNAK | GPSM2 | PRSS23* |
| AHR | GPX3* | PSAP* |
| AK3 | GRHL2 | PSCA* (includes EG: 8000) |
| AKAP10 | GRINA | PSEN1* |
| AKAP12 | GSTA3 | PSME4 |
| ALDH2 | GSTM1 | PSMG2 |
| ALDH1L1 | GSTM2 | PSTPIP2 |
| ALDH3A1 | GSTM4 | PTBP2 |
| ALDOC | GSTM5 | PTOV1 |
| AMN* | GSTM3 (includes EG: 2947) | PTP4A1 |
| AMOT* | GTF2H4 | PTP4A2* |
| ANK1* | GTPBP2 | PTPLAD1 |
| ANKRD43 | H19 | PTPLB |
| ANKRD46 | H1F0 | PTPRB* |
| ANO1 | H2AFV (includes EG: 94239) | PTPRE* |
| ANP32B | H2AFX | PTPRR* |
| ANP32E | HADH | PTPRU* |
| ANTXR1* | HAT1 | PTTG1 |
| ANUBL1 | HBP1 | PVRL2* |
| ANXA3 | HBS1L | PVRL3* |
| ANXA6* | HCK | RAB10 |
| AOF1 | HDAC11 | RAB14 |
| AOX1 | HDGF* | RAD21 |
| AP3B1 | HERPUD1 | RAI14 |
| APBB2 | HEXIM1 | RANGAP1 |
| APCDD1 | HIF1A | RAP2C |
| API5 | HINT3 | RARG |
| APLP2* | HIP1R | RARRES2* |
| APOC1* | HIST1H1C | RASA3* |
| APP* | HMG20A | RASL11B |
| AQP1* | HMGCS2 | RASSF3 |
| AQP3* | HMOX1 | RBL1 |
| ARAF | HN1 | RBM5 |
| ARCN1 | HNRNPR | RBM6 |
| ARF1 | HNRNPU | RBM14 |
| ARF4 | HPGD | RBM39 |
| ARFIP2 | HR | RBMS1 |
| ARHGAP26 | HRSP12 | RBMS3 |
| ARL6 | HS3ST1 | RBMX |
| ARL5B | HSD17B2 | RBP1* |
| ARMCX1 | HSD17B12 | RCAN1 |
| ARMCX3 | HSPA13 | RCBTB2 |
| ARRDC3 | HSPA14 | RDBP |
| ARSA | HTATIP2 | REEP5* |
| ASB8 | HTR4* | RFC4 |
| ASCC2 | HTRA1* | RGS2 |
| ATAD2 | ICOSLG* | RGS5* |
| ATF4 | ID2 | RGS10 |
| ATL2 | ID4 | RHOC* |
| ATP1B1* | IDE* | RHOQ* |
| ATP2A2 | IDH1 | RHOU |
| ATP6V0A1 | IDS | RIPK3* |
| ATRN* | IER3 | RNASE4* |
| ATRX | IGF2* | RNASEH2B |
| BACE1 | IGF1R* | RNF13 |
| BACE2 | IGFBP2* | RNF14 |
| BAZ1A | IGFBP4* | RNF125 |
| BCAM* | IGFBP5* | RNF144B |
| BCL10 | IGFBP6* | RNF19A |
| BCOR | IL18* | RNPS1 |
| BFAR | IL17RE | ROD1 |
| BGN* | IL1RN* | RPA1 |
| BHLHE40 | IL6ST* | RPA3 |
| BLM | IMPA2 | RPP30 |
| BMP2* | IMPACT | RSAD2 |
| BOK | INMT | RTN4* |
| BPTF | INTS6 | RUSC1 |
| BRAF | IPP | S100A1 |
| BRCA1 | IRF1 | S100A10 |
| BRCA2 | IRF2 | S100A14 |
| BRWD1 | IRF8 | SAPS3 |
| BST2* | IRS1 | SATB1 |
| BXDC5 | ITPK1 | SBSN |
| BZW1 | ITPR1 | SCAMP1 |
| C1GALT1* | IVL | SCO1 |
| CACNA1H* | JMJD1C | SCOC |
| CALML3 | JUN | SDC1* |
| CAMK2B | KAT2B | SDC2* |
| CAMK2D | KCNE1* | SDC4* |
| CAMK2N1* | KCNK6* | SDPR* |
| CAP1* | KCTD3 | SEC63 |
| CAPN1 | KCTD15 | SEC61A1 |
| CAPN2 | KDELR3 | SEH1L |
| CAPRIN1* | KDM5D | SELE* |
| CARHSP1 | KDM6A | SEMA4C* |
| CASC4 | KHSRP | SEMA4G* |
| CASK* | KLF4 | SENP8 |
| CASP2 | KLF5 | SERINC1* |
| CASP3 | KLHL22 | SERPING1* |
| CASP7 | KLHL24 | SERPINH1* |
| CAT | KLK6* | SERTAD1 |
| CAV1* | KPNA1 | SESN1 |
| CBX2 | KPNA3 | SETD5 |
| CBX5 | KPNB1 | SF1 |
| CCDC69 | LAPTM4A | SFPQ |
| CCDC80 | LASP1 | SFRS1 |
| CCDC99 | LBH | SFRS2 |
| CCND1 | LBR | SFRS3 |
| CCNG1 | LCN2* | SFRS7 |
| CCNG2 | LDHA | SFRS2IP |
| CCNL2 | LDHB | SGCE* |
| CCPG1 | LGALS1* | SGPL1 |
| CCRN4L | LGALS3* | SGPP1 |
| CD44* | LIMA1 | SH3BGRL |
| CD55* | LIMD1 | SH3GL2* |
| CD99L2* | LIMK2 | SH3GL3 |
| CDC6 | LIMS1* | SHFM1 |
| CDC73 | LIN9 | SHH* |
| CDC37L1 | LIN7C | SHISA4 |
| CDC42EP3 | LMAN2 | SHOC2 |
| CDC45L | LMBR1* | SIDT1 |
| CDCA4 | LMNB1 | SKI |
| CDKN3 | LMO1 | SKP2 |
| CDKN1A | LONRF3 | SLBP |
| CDKN1C | LPIN1 | SLC12A2* |
| CDO1 | LPL | SLC12A6* |
| CELA1* | LPP | SLC12A7* |
| CENPA | LRP1* | SLC16A9 |
| CENPE | LRRC50 | SLC16A12 |
| CFD* | LRRC8E | SLC1A5* |
| CHKA | LSM3 | SLC22A17* |
| CHUK | LSM5 | SLC22A18* |
| CIRBP | LSM8 | SLC23A2* |
| CITED2 | LUC7L2 | SLC24A6* |
| CKAP2L | LUM | SLC25A24 |
| CKB | LY6E | SLC29A1* |
| CKLF* | MAFB | SLC2A1* |
| CLCA2* (includes EG: 9635) | MAGOH | SLC30A1* |
| CLCN5* | MALAT1 | SLC35A5 |
| CLDN8* | MAN1A2 | SLC40A1* |
| CLIC4 | MAP4K5 | SLC44A2* |
| CLTC* | MAPK6 | SLC4A4* |
| CLU* | MAPK14 | SLC4A7* |
| CNN1 | MAPKAPK5 | SLC5A1* |
| CNN2 | MAPRE2 | SLC6A6* |
| CNTNAP2* | MARCKSL1 | SLC9A2* |
| COL18A1* | MAST4 | SLC9A3R2* |
| COL1A2* | MBNL2 | SLCO2A1* |
| COL3A1* | MBTD1 | SMARCA2 |
| COL4A1* | MCM4 | SMARCA4 |
| COL5A1* | MCM6 | SMC4 |
| COL5A2* | MCM10 | SMC6 |
| COL6A1* | MDM4 | SMPDL3B* |
| COPG2 | ME2 | SNAP23* |
| COPZ2 (includes EG: 51226) | MEF2A (includes EG: 4205) | SNAPC3 |
| CORO1C | MEIS1 | SNORD22 (includes EG: 9304) |
| CORO2A | MERTK* | SNRPA1 |
| CPD* | MFAP3L | SNRPD1 |
| CPEB2 | MGP* | SNX6 |
| CPSF3L | MLLT4 | SNX25 |
| CREB3L2 | MLYCD | SOCS3 |
| CREBL2 | MMGT1 | SOD1 |
| CRIM1* | MMP2* | SON |
| CRIP1 | MOBKL1B* | SORL1* |
| CRISPLD2 (includes EG: 83716) | MOCS1 | SORT1 |
| CROT | MPZL1* | SPARC* |
| CRYZ | MPZL2* | SPARCL1* |
| CRYZL1 | MRE11A | SPHK1 |
| CSAD | MRPL9 | SPIN1 |
| CSF1R* | MRPS2 | SPON1* |
| CSNK1A1 | MRPS23 | SPOP* |
| CSNK1E | MSH6 | SPTLC1 |
| CSRP1 | MSI2 | SRI |
| CSTF2 (includes EG: 1478) | MSRB3 (includes EG: 253827) | SRRM2 |
| CTNNB1 | MTM1 | SSRP1 |
| CTNS | MTMR2 | STAG2 |
| CTSD | MTMR4 | STAM2 |
| CTSE | MTMR6 | STAMBP |
| CTTN* | MTSS1 | STARD13 |
| CXADR* | MTUS1 | STOX2 |
| CXCL14* | MXI1 | SUB1 |
| CXCL16* | MXRA8 | SULF1 |
| CYB5B | MYCL1 | SUOX |
| CYB5R3 | MYEF2 | SUPT16H |
| CYFIP2 (includes EG: 26999) | MYH11 | SYNJ2BP |
| CYP1A1 | MYL9 (includes EG: 10398) | SYNM |
| CYP1B1 | MYLIP | SYPL1* |
| DAB1 | MYLK | SYTL2 |
| DAB2* | N6AMT1 | TAF15 |
| DALRD3 | NAB2 | TAGLN |
| DAPK1 | NANS | TAGLN2 |
| DBP | NAP1L1 | TARDBP |
| DCBLD2* | NAPA | TBC1D8* |
| DCN* | NAT11 | TBC1D9* |
| DCTD | NAV1 | TBX3 |
| DCXR | NBEAL1 | TCF4 |
| DDX20 | NBR1 | TCF21 |
| DDX46 | NCAPG2 | TCF25 |
| DECR1 | NCAPH | TCP11L2 |
| DECR2 | NDE1 | TCTA |
| DEFB1* | NDN | TERF1 |
| DEGS1* | NDRG1 | TFF1* |
| DEPDC6 (includes EG: 64798) | NEDD1 | TFRC* |
| DGCR6* | NEDD4 | TGFBI* |
| DNA2 | NEDD4L | TGM2 |
| DNAJA2 | NEK3 | THBS2* |
| DNAJA4 | NET1 | THOC4 |
| DNAJB1 | NFAT5 | THRAP3 |
| DNAJC1 | NFIA | TIMP2* |
| DNAJC12 | NFIB | TIPARP |
| DNM2* | NFIX | TK2 |
| DNM1L | NFKBIA | TLR2 |
| DNMT3L | NFKBIL2 | TMCC3 |
| DOCK8 | NFS1 | TMED2 |
| DOCK11 | NGFRAP1* | TMEM8* |
| DPT* | NIPA1* | TMEM33 |
| DR1 | NIPSNAP1 | TMEM49* |
| DSC2* | NISCH* | TMEM57 |
| DSG2* | NLK | TMEM64 |
| DSP* | NME4 | TMEM146 |
| DTNB* | NONO | TMEM184C |
| DUSP1 | NOTCH1* | TMPO |
| DUSP16 | NQO1 | TMPRSS2* |
| DVL1 | NRARP | TMX4 |
| DYNLT3 | NRIP1 | TNC* |
| DYRK1A | NUBP1 | TNKS1BP1 |
| ECM1* | NUCB2 | TNPO2 |
| EFEMP1* | NUCKS1 | TNS1* |
| EGLN3 | NUDCD1 | TOB2 |
| EGR1 | NUP35 | TOE1 |
| EHF | NUP43 | TOP2B |
| EID1 | NUP54 | TOPBP1 |
| EIF5 | NUP133 | TPI1 |
| EIF2AK1 | NUPL1 | TPM2 |
| EIF2AK2 | NUPR1 | TPMT |
| EIF4A2 | OGT | TPP1 |
| EIF4EBP2 | OLFM1 | TRAK1 |
| EIF4G1 | ORC1L | TRIM2 |
| ELF3 | ORMDL3 | TRIO* |
| ELF5 | OSBPL8 | TRIP13 |
| ELL2 | OSMR* | TSC22D3 |
| ELOVL5 | OTUD4 | TSPAN6* |
| EMD | P2RX4* | TSPAN7* |
| EME1 | PABPN1 | TSPAN8* |
| EMID1* | PACS2 | TSSC4 |
| EMP1* | PAFAH1B2 | TTC1 |
| EMP2* | PAPOLA | TTC14 |
| ENAH | PAQR3 | TWF1 |
| ENPP5* | PAQR5 | TWSG1* |
| ENTPD5 | PAQR6 | TXLNA* (includes EG: 200081) |
| EPHB2* | PAQR7 | TXNIP |
| EPHX1 | PAQR8* | TYMP* |
| EPHX3 | PARD3 | TYRO3* |
| EPPK1 | PARP4 | UBAP2L |
| EPSTI1 | PBX1 | UBE2B |
| ERBB2IP* (includes EG: 55914) | PCGF3 | UBE2H (includes EG: 7328) |
| ERI1 | PCGF6 | UBE4B |
| ERO1L | PCMTD1 | UBTF |
| ERRFI1 | PCMTD2 | UCHL1 |
| EXD1 | PCNA | UGDH |
| EXOSC6 | PCOLCE* | UNC5A* |
| EXT1 | PCYOX1 | UNC84A |
| EZH2 | PDCD6IP | UPK2* |
| EZR* | PDE2A | UPK1B* |
| F3* | PDGFRA* | USP1 |
| F2R* | PDHA1 (includes EG: 5160) | USP15 |
| F2RL1* | PDK4 | USP32 |
| FABP4 | PDLIM5 | USP27X |
| FADS2* | PDZD2* | VAMP3* |
| FAF1 | PDZRN3* | VAMP4 |
| FAR1 | PEG3 | VAPB* |
| FBP1 | PELI1 | VEGFA* |
| FBXL3 | PEX3 | VIM |
| FBXO21 | PFDN4 | VLDLR* |
| FBXW2 | PFDN5 | VNN1* |
| FBXW7 | PFKFB3 | VPS35 |
| FBXW11 | PFKM | VPS41 |
| FERMT2 | PFKP | VRK1 |
| FETUB* | PHF10 | WAC |
| FGFR2* | PHF13 | WBSCR27 |
| FHL1 | PHF17 | WDFY3 |
| FLOT2* | PHKB | WDR1* |
| FMO2 | PHTF2 | WDR5 |
| FMO5 | PI16* | WDR23 |
| FN1* | PI4K2B | WDR77 |
| FOXP2 | PI4KA | WDR40B |
| FRAS1* | PIK3R1 | WEE1 |
| FSTL1* | PIM3 | WHSC1 |
| FUBP1 | PIP4K2C | WNK1 |
| FUSIP1 | PITPNB | WNT5A* |
| FUT9 | PKD2* (includes EG: 5311) | WWTR1 |
| FXYD4 | PKP2* | XIST |
| FZD2* | PLA2G6 | YIPF2 |
| FZD6* | PLA2G7* | YWHAH |
| GADD45A | PLA2R1* | ZBTB20 |
| GADD45B | PLAC8 | ZCCHC7 |
| GALNT6 | PLCH1 | ZDHHC9 |
| GALNT7 | PLDN | ZFAND5 |
| GALNTL1 | PLEKHA2 | ZFAND6 |
| GARNL1 | PLEKHF2 (includes EG: 79666) | ZFP62 |
| GARNL4 | PLK4 | ZFP91 |
| GAS6* | PLS3 | ZFYVE27 |
| GATA6 | PLTP* | ZMYM1 |
| GCNT1 | PMAIP1 | ZMYM6 |
| GCNT2 | PMEPA1* | ZNF638 |
| GEM* | PMP22* | ZNHIT3 |
| GFRA3* | PNN* | |

Note:
Gene products previously reported to be cell surface or secreted proteins denoted with a “*” symbol

TABLE 4

| | | |
|---|---|---|
| ABCA1 | EFEMP1 | PROCR |
| ABCA3 | EMID1 | PSAP |
| ABCC3 | EPHB2 | PSCA (includes EG: 8000) |
| ADAM10 | ERBB2IP (includes EG: 55914) | PSEN1 |
| ADAM28 | EZR | PTPRB |
| ADAMDEC1 | F3 | PVRL2 |
| AMN | F2R | PVRL3 |
| ANK1 | F2RL1 | RARRES2 |
| ANTXR1 | FETUB | RASA3 |
| ANXA6 | FGFR2 | RBP1 |
| APLP2 | FLOT2 | RHOC |
| APOC1 | FN1 | RNASE4 |
| APP | FRAS1 | RTN4 |
| AQP1 | FSTL1 | SDC1 |
| AQP3 | FZD2 | SDC2 |
| ATP1B1 | GAS6 | SDC4 |
| ATRN | GFRA3 | SELE |
| BCAM | GNG10 | SERPING1 |
| BGN | GPC4 | SH3GL2 |
| BST2 | GPX3 | SHH |
| CACNA1H | HTR4 | SLC12A2 |
| CAP1 | ICOSLG | SLC12A6 |
| CAPRIN1 | IGF2 | SLC12A7 |
| CASK | IGF1R | SLC1A5 |
| CAV1 | IGFBP2 | SLC29A1 |
| CD44 | IGFBP4 | SLC2A1 |
| CD55 | IGFBP5 | SLC40A1 |
| CD99L2 | IGFBP6 | SLC44A2 |
| CELA1 | IL18 | SLC4A7 |
| CFD | IL1RN | SLC5A1 |
| CKLF | IL6ST | SNAP23 |
| CLTC | KCNE1 | SORL1 |
| CLU | KLK6 | SPARC |
| CNTNAP2 | LCN2 | SPARCL1 |
| COL18A1 | LGALS1 | SYPL1 |
| COL1A2 | LGALS3 | TFF1 |
| COL3A1 | LIMS1 | TFRC |
| COL4A1 | LRP1 | TGFBI |
| COL5A1 | MERTK | THBS2 |
| COL5A2 | MGP | TIMP2 |
| COL6A1 | MMP2 | TMEM8 |
| CPD | NISCH | TMPRSS2 |
| CSF1R | NOTCH1 | TNC |
| CTTN | P2RX4 | TRIO |
| CXCL16 | PCOLCE | TWSG1 |
| DAB2 | PDGFRA | TXLNA (includes EG: 200081) |
| DEFB1 | PDZD2 | TYMP |
| DNM2 | PI16 | TYRO3 |
| DPT | PLA2G7 | UPK2 |
| DSC2 | PLA2R1 | VAMP3 |
| DSG2 | PLTP | VEGFA |
| DSP | PNN | VLDLR |
| ECM1 | PON3 | VNN1 |

TABLE 5

| | | |
|---|---|---|
| ABCA1 | EPHB2 | PSEN1 |
| ABCA3 | ERBB2IP (includes EG: 55914) | PTPRB |
| ABCC3 | EZR | PVRL2 |
| ADAM10 | F2R | PVRL3 |
| ADAM28 | F2RL1 | RARRES2 |
| ADAMDEC1 | FETUB | RASA3 |
| AMN | FGFR2 | RBP1 |
| ANK1 | FLOT2 | RHOC |
| ANTXR1 | FRAS1 | RTN4 |
| ANXA6 | FSTL1 | SDC1 |
| APLP2 | FZD2 | SDC2 |
| APOC1 | GAS6 | SDC4 |
| APP | GFRA3 | SELE |
| AQP1 | GNG10 | SERPING1 |
| AQP3 | GPC4 | SH3GL2 |
| ATP1B1 | GPX3 | SHH |
| ATRN | HTR4 | SLC12A2 |
| BCAM | ICOSLG | SLC12A6 |
| BGN | IGF1R | SLC12A7 |
| BST2 | IGFBP2 | SLC1A5 |
| CACNA1H | IGFBP4 | SLC29A1 |
| CAP1 | IGFBP6 | SLC2A1 |
| CAPRIN1 | IL18 | SLC40A1 |
| CASK | IL1RN | SLC44A2 |
| CD44 | IL6ST | SLC4A7 |
| CD55 | KCNE1 | SLC5A1 |
| CD99L2 | KLK6 | SNAP23 |
| CELA1 | LCN2 | SORL1 |
| CFD | LGALS3 | SPARC |
| CKLF | LIMS1 | SYPL1 |
| CLTC | LRP1 | TFF1 |
| CLU | MERTK | TFRC |
| CNTNAP2 | MMP2 | TGFBI |
| COL18A1 | NISCH | THBS2 |
| COL4A1 | NOTCH1 | TIMP2 |
| COL5A1 | P2RX4 | TMEM8 |
| CPD | PCOLCE | TMPRSS2 |
| CSF1R | PDZD2 | TRIO |
| CXCL16 | PI16 | TWSG1 |
| DAB2 | PLA2G7 | TXLNA (includes EG: 200081) |
| DEFB1 | PLA2R1 | TYMP |
| DNM2 | PLTP | TYRO3 |
| DSC2 | PNN | UPK2 |
| DSG2 | PON3 | VAMP3 |
| DSP | PROCR | VEGFA |
| ECM1 | PSAP | VLDLR |
| EFEMP1 | PSCA (includes EG: 8000) | VNN1 |
| EMID1 | | |

TABLE 6
(ELISA, values in U/ml)

| Patient | visit | AMFR | RacGap | RHAMM | PCNA* |
|---|---|---|---|---|---|
| 1754641 | BASELINE | 29.12 | 0.74 | 9.31 | 0.00 |
| 1754641 | EARLY | 32.57 | 0.42 | 10.27 | 0.00 |
| | TERM | | | | |
| 1264555 | BASELINE | 9.30 | 0.02 | 0.98 | 0.40 |
| 344121 | BASELINE | 1.65 | 0.00 | 0.64 | 0.02 |
| 344121 | VISIT 4 | 3.18 | 0.05 | 1.91 | 0.00 |
| 344121 | VISIT 6 | 3.20 | 0.06 | 4.29 | 0.00 |
| 1334562 | BASELINE | 1.18 | 1.07 | 1.20 | 0.00 |
| 1334562 | VISIT 4 | 3.97 | 0.53 | 2.06 | 0.00 |
| 1334562 | VISIT 6 | 23.73 | 0.44 | 3.64 | 0.02 |
| 724459 | BASELINE | 22.53 | 2.31 | 19.20 | 0.00 |
| 724459 | VISIT 4 | 8.77 | 0.34 | 5.08 | 0.00 |
| 724459 | VISIT 6 | 1.60 | 0.65 | 5.68 | 0.00 |
| 674475 | BASELINE | 20.46 | 1.85 | 9.48 | 0.00 |
| 674475 | VISIT 4 | 13.86 | 0.49 | 0.32 | 0.00 |
| 674475 | VISIT 6 | 2.61 | 0.50 | 0.00 | 0.00 |
| 674475 | EARLY | 35.34 | 4.47 | 8.98 | 0.00 |
| | TERM | | | | |
| 1254563 | BASELINE | 14.02 | 1.40 | 0.93 | 0.00 |
| 1254563 | VISIT 4 | 1.13 | 0.38 | 0.00 | 0.00 |
| 1124730 | BASELINE | 12.32 | 1.40 | 6.79 | 0.00 |
| 1554559 | BASELINE | 13.57 | 0.45 | 14.24 | 0.00 |
| 1554559 | VISIT 4 | 8.22 | 0.64 | 9.46 | 0.00 |
| 1424566 | BASELINE | 5.89 | 0.15 | 0.24 | 0.14 |
| 1424566 | VISIT 4 | 21.70 | 0.59 | 1.53 | 0.19 |
| 1424566 | EARLY | 14.23 | 1.00 | 0.41 | 0.18 |
| | TERM | | | | |
| 574382 | BASELINE | 3.11 | 0.42 | 13.45 | 0.50 |
| 574382 | EARLY | 27.29 | 0.27 | 3.78 | 0.35 |
| | TERM | | | | |
| 584016 | BASELINE | 10.25 | 0.06 | 2.86 | 0.21 |
| 584016 | EARLY | 1.97 | 0.17 | 5.37 | 0.22 |
| | TERM | | | | |
| 1504628 | BASELINE | 5.12 | 0.85 | 2.44 | 0.15 |
| 1504628 | EARLY | 2.14 | 1.36 | 11.27 | 0.10 |
| | TERM | | | | |
| 1414602 | BASELINE | 34.16 | 0.37 | 0.08 | 0.24 |
| 1414602 | VISIT 4 | 10.95 | 0.60 | 16.33 | 0.18 |
| 1414602 | VISIT 6 | 0.88 | 0.76 | 4.10 | 0.05 |
| 364075 | BASELINE | 3.37 | 0.02 | 2.54 | 0.51 |
| 364075 | VISIT 4 | 1.03 | 0.00 | 1.63 | 0.17 |
| 364075 | EARLY | 22.06 | 0.44 | 0.88 | 0.00 |
| | TERM | | | | |
| 1474540 | BASELINE | 32.65 | 0.36 | 4.16 | 0.11 |
| 1474540 | VISIT 4 | 2.97 | 0.13 | 0.87 | 0.20 |
| 1474540 | VISIT 6 | 24.72 | 0.58 | 6.13 | 0.19 |
| 624329 | BASELINE | 6.19 | 0.06 | 0.59 | 0.16 |
| 624329 | VISIT 4 | 3.63 | 0.06 | 0.49 | 0.23 |
| 624329 | VISIT 6 | 7.60 | 0.49 | 1.40 | 0.37 |
| 984652 | BASELINE | 7.93 | 0.54 | 1.79 | 0.09 |
| 984652 | VISIT 4 | 6.46 | 0.35 | 1.77 | 0.04 |
| 684479 | BASELINE | 7.58 | 0.35 | 2.27 | 0.24 |
| 684479 | VISIT 4 | 9.23 | 0.52 | 2.53 | 0.96 |
| 684479 | VISIT 6 | 1.57 | 0.36 | 0.12 | 0.88 |
| 684479 | EARLY | 1.44 | 0.16 | 1.06 | 0.61 |
| | TERM | | | | |
| 694457 | BASELINE | 18.04 | 0.30 | 0.68 | 0.21 |
| 694457 | VISIT 4 | 1.46 | 0.04 | 0.32 | 0.35 |
| 694457 | VISIT 6 | 2.31 | 0.03 | 0.10 | 0.18 |
| 714474 | BASELINE | 6.24 | 0.06 | 0.13 | 0.00 |
| 714474 | VISIT 4 | 0.00 | 0.00 | 0.17 | 0.00 |
| 714474 | VISIT 6 | 40.73 | 0.65 | 1.61 | 0.07 |
| 804330 | BASELINE | 23.02 | 0.08 | 0.42 | 0.23 |
| 804330 | VISIT 4 | 12.22 | 0.04 | 0.19 | 0.51 |
| 804330 | EARLY | 33.93 | 0.18 | 0.00 | 0.26 |
| | TERM | | | | |
| 924546 | BASELINE | 7.51 | 0.04 | 0.94 | 1.22 |
| 924546 | VISIT 4 | 1.99 | 0.05 | 0.64 | 0.83 |
| 924546 | VISIT 6 | 1.78 | 0.04 | 1.21 | 1.12 |
| 1514504 | BASELINE | 5.75 | 0.25 | 6.42 | 0.99 |
| 1514504 | VISIT 4 | 1.82 | 0.02 | 0.64 | 0.80 |
| 1514504 | EARLY | 3.50 | 0.51 | 6.52 | 0.86 |
| | TERM | | | | |
| 174073 | BASELINE | 6.05 | 0.30 | 4.26 | 0.71 |
| 174073 | VISIT 4 | 1.24 | 0.13 | 1.53 | 0.74 |
| 174073 | VISIT 6 | 3.12 | 0.19 | 1.17 | 0.62 |
| 254017 | BASELINE | 0.98 | 0.04 | 0.94 | 0.86 |
| 254017 | VISIT 4 | 6.11 | 0.08 | 2.59 | 0.77 |
| 254017 | VISIT 6 | 10.75 | 0.23 | 8.75 | 0.79 |
| 424441 | BASELINE | 2.32 | 0.15 | 1.57 | 0.71 |
| 424441 | VISIT 4 | 0.35 | 0.19 | 2.39 | 1.02 |
| 424441 | VISIT 6 | 0.19 | 0.15 | 2.28 | 1.18 |
| 74012 | BASELINE | 0.58 | 0.15 | 2.49 | 0.60 |
| 74012 | VISIT 4 | 0.88 | 0.11 | 1.35 | 0.74 |
| 74012 | VISIT 6 | 0.18 | 0.04 | 0.60 | 0.73 |
| 124014 | BASELINE | 4.17 | 0.10 | 4.75 | 0.06 |
| 124014 | VISIT 4 | 3.51 | 0.13 | 6.46 | 0.05 |
| 634417 | BASELINE | 0.19 | 0.13 | 1.67 | 1.10 |
| 634417 | VISIT 4 | 1.27 | 0.15 | 3.26 | 0.77 |
| 634417 | VISIT 6 | 4.93 | 0.41 | 4.98 | 0.63 |
| 634417 | EARLY | 2.45 | 0.65 | 6.57 | 0.81 |
| | TERM | | | | |
| 664418 | BASELINE | 3.81 | 0.00 | 1.41 | 0.75 |
| 664418 | VISIT 4 | 2.50 | 0.08 | 0.95 | 1.42 |
| 664418 | VISIT 6 | 6.82 | 0.16 | 1.01 | 1.02 |
| 704332 | BASELINE | 12.31 | 0.21 | 0.29 | 0.85 |
| 704332 | VISIT 4 | 0.00 | 0.00 | 0.04 | 0.83 |
| 704332 | VISIT 6 | 6.02 | 0.08 | 0.34 | 0.95 |
| 1414602 | BASELINE | 1.22 | 0.00 | 0.04 | 0.24 |
| 1414602 | VISIT 4 | 7.30 | 0.05 | 0.08 | 0.18 |
| 1414602 | VISIT 6 | 13.83 | 0.04 | 0.17 | 0.05 |
| 14001 | BASELINE | 9.85 | 0.06 | 0.79 | 0.56 |
| 14001 | VISIT 4 | 1.05 | 0.04 | 0.13 | 0.55 |
| 14001 | VISIT 6 | 3.07 | 0.07 | 0.86 | 0.62 |
| 24002 | BASELINE | 0.77 | 0.00 | 0.00 | 0.92 |
| 24002 | VISIT 4 | 5.91 | 0.00 | 0.73 | 1.02 |
| 24002 | VISIT 6 | 1.25 | 0.00 | 0.00 | 3.27 |
| 1584532 | BASELINE | 0.67 | 0.02 | 1.66 | 0.79 |
| 1584532 | EARLY | 2.75 | 0.21 | 2.09 | 0.79 |
| | TERM | | | | |
| 414082 | BASELINE | 13.19 | 0.49 | 3.69 | 0.65 |
| 414082 | VISIT 4 | 1.12 | 0.04 | 0.59 | 0.64 |
| 414082 | VISIT 6 | 3.63 | 0.26 | 1.12 | 0.79 |
| 194066 | BASELINE | 1.11 | 0.07 | 0.89 | 0.57 |
| 224070 | BASELINE | 3.49 | 0.16 | 2.52 | 1.17 |
| 164065 | BASELINE | 11.10 | 0.37 | 5.38 | 0.33 |
| 1494552 | BASELINE | 0.00 | 0.00 | 0.00 | 0.82 |
| 1494552 | VISIT 4 | 2.64 | 0.08 | 0.46 | 0.90 |
| 1234754 | BASELINE | 4.93 | 0.13 | 0.75 | 0.74 |
| 1234754 | VISIT 4 | 4.68 | 0.25 | 0.85 | 1.24 |
| 1234754 | VISIT 6 | 11.00 | 0.55 | 3.68 | 0.74 |
| 1004538 | BASELINE | 27.79 | 1.46 | 6.00 | 0.14 |
| 864651 | BASELINE | 6.51 | 0.42 | 4.58 | 1.44 |
| 864651 | VISIT 4 | 0.41 | 0.03 | 0.43 | 0.73 |
| 864651 | VISIT 6 | 0.89 | 0.04 | 1.21 | 0.84 |
| 734667 | BASELINE | 4.94 | 0.33 | 4.06 | 0.64 |
| 734667 | VISIT 4 | 9.92 | 0.79 | 4.72 | 0.76 |
| 734667 | VISIT 6 | 21.99 | 2.41 | 20.47 | 1.32 |
| 744460 | BASELINE | 3.12 | 0.11 | 2.34 | 1.24 |
| 744460 | EARLY | 9.09 | 0.08 | 4.93 | 1.02 |
| | TERM | | | | |
| 614380 | BASELINE | 3.34 | 0.02 | 0.20 | 1.19 |
| 614380 | VISIT 4 | 0.33 | 0.01 | 0.00 | 1.26 |
| 614380 | VISIT 10 | 1.61 | 0.12 | 1.81 | 0.79 |
| 614380 | VISIT 6 | 0.07 | 0.02 | 0.15 | 1.03 |
| 1774764 | BASELINE | 9.52 | 1.29 | 8.08 | 0.86 |
| 1774764 | EARLY | 3.62 | 0.95 | 1.52 | 0.72 |
| | TERM | | | | |
| 534316 | BASELINE | 8.69 | 0.20 | 5.02 | 0.99 |
| 534316 | VISIT 4 | 7.46 | 0.19 | 2.98 | 0.80 |
| 534316 | VISIT 6 | 9.07 | 0.24 | 5.38 | 0.86 |
| 454076 | BASELINE | 4.82 | 0.19 | 11.40 | 1.49 |
| 454076 | VISIT 4 | 2.69 | 0.12 | 14.71 | 0.99 |
| 454076 | VISIT 6 | 19.01 | 0.16 | 10.86 | 0.68 |
| 494313 | BASELINE | 9.03 | 0.05 | 6.21 | 1.19 |
| 494313 | VISIT 4 | 5.98 | 0.10 | 14.15 | 0.93 |
| 494313 | VISIT 6 | 3.56 | 0.04 | 9.65 | 0.89 |
| 1714661 | BASELINE | 1.75 | 0.23 | 1.48 | 0.88 |
| 1714661 | VISIT 4 | 20.52 | 1.34 | 5.45 | 1.54 |
| 1714661 | EARLY | 9.40 | 0.64 | 3.41 | 0.84 |
| | TERM | | | | |
| 1194537 | BASELINE | 7.77 | 0.48 | 2.38 | 1.39 |
| 1194537 | VISIT 4 | 5.25 | 0.47 | 12.08 | 1.06 |
| 1194537 | VISIT 6 | 1.82 | 0.04 | 1.95 | 0.93 |
| 1724660 | BASELINE | 0.84 | 0.00 | 0.00 | 0.98 |
| 1724660 | VISIT 4 | 6.18 | 0.49 | 7.34 | 1.07 |
| 1724660 | EARLY | 1.79 | 0.13 | 1.46 | 1.23 |
| | TERM | | | | |
| 464162 | BASELINE | 1.08 | 0.23 | 1.23 | 0.84 |
| 1174556 | BASELINE | 1.79 | 0.36 | 8.57 | 0.97 |
| 1174556 | EARLY | 1.11 | 0.00 | 1.51 | 0.76 |
| | TERM | | | | |
| 1214554 | BASELINE | 6.21 | 0.40 | 2.95 | 0.74 |
| 1214554 | VISIT 4 | 1.55 | 0.80 | 1.99 | 0.62 |
| 1214554 | VISIT 6 | 0.16 | 0.06 | 0.33 | 0.86 |
| 1214554 | EARLY | 0.21 | 0.00 | 0.44 | 0.74 |
| | TERM | | | | |
| 234067 | BASELINE | 0.58 | 0.41 | 1.42 | 0.62 |
| 234067 | VISIT 4 | 3.31 | 0.95 | 4.77 | 0.77 |
| 1464557 | BASELINE | 11.83 | 0.39 | 1.24 | 0.87 |
| 1464557 | VISIT 4 | 7.32 | 0.16 | 1.14 | 0.86 |
| 1464557 | VISIT 6 | 2.09 | 0.13 | 1.31 | 1.58 |
| 1464557 | EARLY | 47.80 | 1.34 | 7.53 | 0.93 |
| | TERM | | | | |
| 354129 | BASELINE | 0.37 | 0.03 | 0.28 | 1.09 |
| 354129 | EARLY | 9.75 | 0.50 | 3.72 | 0.66 |
| | TERM | | | | |
| 1384501 | BASELINE | 0.30 | 0.09 | 0.45 | 0.83 |
| 1384501 | VISIT 4 | 0.64 | 0.19 | 0.24 | 1.29 |
| 1384501 | VISIT 6 | 0.96 | 0.47 | 0.29 | 1.41 |
| 704332 | BASELINE | 7.55 | 1.31 | 4.07 | 0.85 |
| 704332 | VISIT 4 | 2.13 | 1.11 | 1.38 | 0.83 |
| 704332 | VISIT 6 | 1.61 | 0.44 | 1.03 | 0.95 |
| 514461 | BASELINE | 1.39 | 0.16 | 1.46 | 0.20 |
| 514461 | VISIT 4 | 11.78 | 0.27 | 1.69 | 0.31 |
| 514461 | EARLY | 9.04 | 0.27 | 1.21 | 0.74 |
| | TERM | | | | |
| 504282 | BASELINE | 3.35 | 0.10 | 0.55 | 0.93 |
| 504282 | VISIT 4 | 7.28 | 0.53 | 1.62 | 0.85 |
| 504282 | VISIT 6 | 8.55 | 0.24 | 2.81 | 0.89 |
| 604384 | BASELINE | 1.29 | 0.61 | 3.71 | 1.00 |
| 604384 | VISIT 4 | 1.49 | 0.46 | 5.19 | 1.14 |
| 604384 | VISIT 6 | 2.00 | 0.55 | 6.54 | 0.92 |
| 604384 | VISIT 10 | 0.32 | 0.07 | 1.91 | 1.24 |
| 44010 | BASELINE | 1.17 | 0.34 | 3.81 | 1.12 |
| 44010 | VISIT 4 | 1.85 | 0.44 | 7.15 | 0.93 |
| 44010 | VISIT 6 | 1.75 | 0.36 | 7.29 | 0.97 |
| 1564560 | BASELINE | 1.23 | 0.13 | 7.57 | 1.05 |
| 1564560 | VISIT 4 | 5.35 | 0.30 | 10.77 | 0.94 |
| 1564560 | VISIT 6 | 1.76 | 0.01 | 1.45 | 0.83 |
| 1464557 | BASELINE | 10.10 | 0.03 | 1.34 | |
| 1464557 | VISIT 4 | 1.62 | 0.00 | 1.94 | |
| 1464557 | VISIT 6 | 0.93 | 0.02 | 0.45 | |
| 1464557 | EARLY | 5.19 | 0.75 | 15.41 | |
| | TERM | | | | |
| 354129 | BASELINE | 0.90 | 0.14 | 0.62 | |
| 354129 | EARLY | 4.36 | 0.18 | 4.76 | |
| | TERM | | | | |
| 1214554 | BASELINE | 7.10 | 0.24 | 5.64 | |
| 1214554 | VISIT 4 | 9.90 | 0.48 | 3.94 | |
| 1214554 | VISIT 6 | 1.40 | 0.03 | 1.26 | |
| 1214554 | EARLY | 0.44 | 0.06 | 1.23 | |
| | TERM | | | | |
| 1174556 | BASELINE | 10.80 | 0.43 | 6.94 | |
| 1174556 | EARLY | 4.91 | 0.03 | 0.60 | |
| | TERM | | | | |
| 1594673 | BASELINE | 1.42 | 0.26 | 1.49 | |
| 1594673 | EARLY | 0.22 | 0.02 | 2.13 | |
| | TERM | | | | |
| 974547 | BASELINE | 1.20 | 0.35 | 3.51 | |
| 974547 | VISIT 4 | 5.55 | 0.12 | 4.53 | |
| 974547 | EARLY | 10.94 | 0.27 | 40.32 | |
| | TERM | | | | |
| 1064548 | BASELINE | 8.46 | 0.20 | 15.16 | |
| 1644712 | BASELINE | 4.60 | 0.31 | 30.45 | |
| 644163 | BASELINE | 10.48 | 0.25 | 30.84 | |
| 644163 | VISIT 4 | 1.08 | 0.09 | 12.70 | |
| 644163 | VISIT 6 | 1.12 | 0.02 | 12.77 | |
| 264068 | BASELINE | 5.26 | 0.21 | 29.30 | |
| 934578 | BASELINE | 5.35 | 0.25 | 16.97 | |
| 934578 | VISIT 4 | 1.50 | 0.07 | 7.31 | |
| 934578 | VISIT 6 | 3.47 | 0.24 | 13.23 | |
| 314074 | BASELINE | 9.01 | 4.16 | 12.96 | |
| 314074 | VISIT 4 | 7.73 | 2.07 | 23.12 | |
| 314074 | VISIT 6 | 6.65 | 0.73 | 34.34 | |
| 204007 | BASELINE | 16.72 | 4.60 | 40.41 | |
| 204007 | VISIT 4 | 4.19 | 0.42 | 41.51 | |
| 204007 | VISIT 6 | 5.71 | 1.03 | 54.74 | |
| 754476 | BASELINE | 0.76 | 0.67 | 22.90 | |
| 1374564 | BASELINE | 0.02 | 0.18 | 1.07 | |
| 1374564 | VISIT 4 | 0.30 | 0.20 | 1.15 | |
| 844507 | BASELINE | 2.25 | 0.64 | 30.29 | |
| 64011 | BASELINE | 7.72 | 1.22 | 21.90 | |
| 64011 | EARLY | 4.52 | 0.40 | 6.82 | |
| | TERM | | | | |
| 1454550 | BASELINE | 1.58 | 0.80 | 1.89 | |
| 1454550 | EARLY | 2.88 | 0.90 | 3.71 | |
| | TERM | | | | |
| 1604793 | BASELINE | 3.15 | 1.12 | 5.68 | |
| 1604793 | VISIT 4 | 2.38 | 1.20 | 5.17 | |
| 1614551 | BASELINE | 3.85 | 0.60 | 3.09 | |
| 274234 | BASELINE | 4.51 | 1.22 | 8.14 | |
| 1094654 | BASELINE | 1.00 | 0.54 | 2.48 | |
| 1094654 | VISIT 4 | 1.81 | 0.76 | 6.11 | |
| 1094654 | VISIT 6 | 1.24 | 0.52 | 6.09 | |
| 54006 | BASELINE | 4.09 | 2.03 | 11.16 | |
| 54006 | VISIT 4 | 1.51 | 0.70 | 2.74 | |
| 54006 | VISIT 6 | 0.29 | 0.21 | 0.86 | |
| 84081 | BASELINE | 2.38 | 0.44 | 3.30 | |
| 84081 | VISIT 4 | 4.67 | 1.88 | 9.06 | |
| 84081 | VISIT 6 | 2.66 | 1.10 | 2.16 | |
| 384083 | BASELINE | 0.61 | 0.33 | 1.08 | |
| 384083 | VISIT 4 | 1.87 | 0.60 | 1.91 | |
| 334131 | BASELINE | 3.68 | 0.58 | 3.82 | |
| 1704761 | BASELINE | 3.74 | 0.88 | 5.04 | |
| 1434706 | BASELINE | 3.80 | 1.19 | 2.18 | |
| 1434706 | EARLY | 0.70 | 0.19 | 1.06 | |
| | TERM | | | | |
| 964625 | BASELINE | 1.19 | 0.40 | 0.88 | |
| 964625 | EARLY | 1.34 | 0.48 | 1.56 | |
| | TERM | | | | |
| 834649 | BASELINE | 1.03 | 0.49 | 1.52 | |
| 834649 | VISIT 6 | 2.83 | 0.87 | 4.29 | |
| 94042 | BASELINE | 1.12 | 0.24 | 0.62 | |
| 94042 | VISIT 4 | 1.72 | 0.63 | 1.40 | |
| 884633 | VISIT 4 | 2.76 | 1.38 | 3.26 | |
| 884633 | VISIT 6 | 3.39 | 1.18 | 3.83 | |
| 1634577 | BASELINE | 21.09 | 0.42 | 6.76 | |
| 1744558 | BASELINE | 33.76 | 0.78 | 14.15 | |
| 1744558 | VISIT 6 | 24.90 | 0.53 | 8.29 | |
| 1744558 | EARLY | 39.77 | 0.75 | 15.31 | |
| | TERM | | | | |
| 1074553 | BASELINE | 6.35 | 0.10 | 1.79 | |
| 1074553 | VISIT 6 | 65.84 | 0.93 | 20.57 | |
| 1784817 | BASELINE | 67.22 | 1.03 | 22.22 | |
| 1784817 | VISIT 4 | 49.11 | 0.80 | 20.62 | |
| 1104561 | BASELINE | 14.50 | 0.49 | 4.31 | |
| 1104561 | EARLY | 6.32 | 0.13 | 2.55 | |
| | TERM | | | | |
| 654458 | BASELINE | 35.77 | 0.52 | 14.33 | |

We claim:

1. A non-invasive or minimally invasive diagnostic or prognostic method for the detection of bladder cancer comprising: (a.) identifying and quantifying an expression level of one or more of the gene products identified in Table 1 in the body fluids of a patient; and (b.) comparing the expression level of the one or more gene products to the expression levels of the one or more gene products found in subjects that do not have bladder cancer.

2. The method of claim 1, wherein the body sample is urine.

3. The method of claim 1, wherein the body sample is blood.

4. The method of claim 1, wherein the one or more gene products identified and quantified in the body fluids of the patient are selected from the group consisting of IL18, PON3, IGF1R, VEGFA, CD44, COL4A1, Hmmr/RHAMM, BIRC5, and PCNA.

5. The method of claim 1, wherein the one or more gene products identified and quantified in the body fluids of the patient are selected from the group consisting of COL5A1, FGFR2, FRAS1, IGF1R, MMP2, SPARC, TGFB1, TIMP2, and UPK2.

6. The method of claim 1, wherein identification and quantification of the expression level is carried out by quantitative or semi-quantitative PCR, ELISA, or dot blot Western blotting.

7. A non-invasive or minimally invasive diagnostic or prognostic method for the detection of bladder cancer comprising: (a.) identifying and quantifying an expression level of a first gene product in the body fluids of a patient, wherein the first gene product is selected from the group consisting of IL18, PON3, IGF1R, VEGFA, CD44, COL4A1, Hmmr/RHAMM, BIRC5, and PCNA; (b.) identifying and quantifying an expression level of a second gene product in the body fluids of a patient, wherein the second gene product is selected from the group consisting of COL5A1, FGFR2, FRAS1, IGF1R, MMP2, SPARC, TGFB1, TIMP2, and UPK2 ; and (c.) comparing the expression levels of the first and second gene products found in the body fluids of the patient to the expression levels of the first and second gene products found in subjects that do not have bladder cancer.

8. The method of claim 7, wherein the body sample is urine.

9. The method of claim 7, wherein the body sample is blood.

10. The method of claim 7, wherein identification and quantification of the expression level is carried out by quantitative or semi-quantitative PCR, ELISA, or dot blot Western blotting.

11. A non-invasive or minimally invasive diagnostic or prognostic method to detect premalignant bladder cancer comprising: (a.) identifying and quantifying an expression level of one or more of the gene products identified in a gene network from Table 2 in the body fluids of a patient; and (b.) comparing the expression level of the one or more of the gene products found in the body fluids of the patient to the expression levels the one or more of the gene products found in subjects that do not have bladder cancer.

12. The method of claim 11, wherein the body sample is urine.

13. The method of claim 11, wherein the body sample is blood.

14. The method of claim 11, wherein the one or more gene products identified and quantified in the body fluids of the patient are selected from the group consisting of JUN, ERK, P21, AURKA, AURKB, ATAD2, CDC7, CDC45L, KIF1C1, and CEP55.

15. The method of claim 11, wherein identification and quantification of the expression level is carried out by quantitative or semi-quantitative PCR, ELISA, or dot blot Western blotting.
